(19) United States
(12) Patent Application Publication
(10) Pub. No.: US 2025/0122357 A1
(43) Pub. Date: Apr. 17, 2025

(54) STABILIZERS BASED ON SYRINGIC ACID, VANILLIC ACID, ISOVANILLIC ACID OR 5-HYDROXYVERATRIC ACID, PLASTIC COMPOSITION, METHOD FOR STABILIZING A PLASTIC COMPOSITION AND STABILIZER COMPOSITION

(71) Applicant: FRAUNHOFER-GESELLSCHAFT ZUR FÖRDERUNG DER ANGEWANDTEN FORSCHUNG E.V., München (DE)

(72) Inventors: Rudolf PFAENDNER, Darmstadt (DE); Elke METZSCH-ZILLIGEN, Darmstadt (DE); Katrin MARKUS, Darmstadt (DE)

(73) Assignee: FRAUNHOFER-GESELLSCHAFT ZUR FÖRDERUNG DER ANGEWANDTEN FORSCHUNG E.V., München (DE)

(21) Appl. No.: 18/708,977

(22) PCT Filed: Nov. 9, 2022
(86) PCT No.: PCT/EP2022/081320

§ 371 Date: May 9, 2024

(30) Foreign Application Priority Data
Nov. 11, 2021 (DE) 10 2021 212 696.0

(51) Int. Cl. C08K 5/134; C08K 5/20

(52) U.S. Cl. CPC C08K 5/1345; C08K 5/20

(57) ABSTRACT

The present invention relates to stabilisers based on syringic acid, vanillic acid, isovanillic acid or 5-hydroxyveratric acid, plastic composition. The invention also relates to a method for stabilising a plastic composition and a stabiliser composition having a high stabilising effect.

This invention relates to stabilizers based on syringic acid, vanillic acid, isovanillic acid, or 5-hydroxy veratric acid, a plastic composition, a process for stabilizing a plastic composition, as well as a stabilizer composition with high stabilizing effect.

Organic materials such as plastics are subject to aging processes which ultimately lead to loss of the desired properties, such as, e.g., the mechanical characteristics. This process, called autoxidation, starts from radical chain cleavage caused by mechanochemical processes or UV radiation in the presence of oxygen, and leads to changes in the polymer chain, such as, e.g., molecular weight and/or the formation of new chemical groups. Therefore, stabilizers are used to prevent or at least delay this aging. Important representatives of stabilizers are antioxidants, which interfere with the radicals formed during autoxidation, and thus interrupt the degradation process. A distinction is generally made between primary antioxidants, which can directly react with oxygen-containing free radicals or C radicals, and secondary antioxidants, which react with hydroperoxides formed as intermediates (see C. Kröhnke et al. Antioxidants in Ullmann's encyclopedia of industrial chemistry, Wiley-VCH Verlag, Weinheim 2015, see appendix). Typical representatives of primary antioxidants are, for example, phenolic antioxidants, amines, but also lactones. Classes of secondary antioxidants are phosphorus compounds such as, e.g., phosphites and phosphonites, but also organosulfur compounds, such as, e.g., thioesters, thioethers, and disulfides. In practice, primary and secondary antioxidants are usually combined, which has a synergistic effect.

To an increasing extent, plastics made from fossil raw materials such as petroleum or natural gas are being supplemented or replaced, through biotechnological processes, by plastics based on renewable raw materials. Then, the question of sustainability also favors the primary and secondary antioxidants used for them (and for plastics from fossil raw materials). Therefore, there is a need for stabilizers based on renewable and available raw materials with high efficacy, low volatility, and compatibility with polymer substrates. Ideally, the antioxidants also have a protective effect with respect to photooxidation to protect polymers used outside.

### PRIOR ART

In principle, antioxidants are known that are made from renewable raw materials and that are also added to plastics in isolated cases. Tocopherols (vitamin E) represent a typical example. Like usual antioxidants, tocopherols have a sterically hindered phenol structure and can be used alone or in combination with secondary antioxidants (e.g., S. Al-Malaika, Macromol. Symp. 2001, 176, 107). Tocopherols are isolated from natural substances such as, e.g., wheat germ oil, sunflower oil, or olive oil. Other known phenolic antioxidants from natural substance that have been studied in plastics are described, for example, in the following literature:
Quercetin (B. Kirschweng et al., Melt stabilisation of PE with natural antioxidants: Comparison of rutin and quercetin, Eur. Pol. J. 2018, 103, 228-237), Dihydromyrecetin (B. Kirschweng et al., Melt stabilization of polyethylene with dihydromyrecitin, a natural antioxidant, Pol. Degr. Stab. 2016, 133, 192-200),
Derivatives of rosmarinic acid (K. Doudin et al., New genre of antioxidants from renewable natural resources: Synthesis and characterisation of rosemary plant-derived antioxidants and their performance in polyolefins, Pol. Degr. Stab. 0.2016, 130, 126-134),
Tannin (W. J. Grigsby et al., Esterification of condensed tannins and their impact on the properties of poly (lactic acid), Polymers 5 (2013) 344-360),
Curcumin (D. Tatraaljai et al. Processing stabilisation of PE with a natural antioxidant, curcumin, European Polymer Journal 49 (2013) 1196-1203),
Silymarin and Sylibin (B. Kirschweng et al., Melt stabilization of polyethylene with natural antioxidants: comparison of a natural extract and its main components, Journal of Thermal Analysis and Calorimetry https://doi.org/10.1007/s10973-020-09709-5),
Catechin from tea and coffee extracts (0. Olejnik, A Masek, Bio-Based Packaging Materials Containing Substances Derived from Coffee and Tea Plants, Materials 2020, 13, 5719).

Furthermore, the authors' own applications for stabilization of plastics with long-chain esters of ferulic acid (WO 2021/191078 A1) and ferulic acid salts (WO 2021/191364 A1) should be mentioned.

J. H. Swisher et al., Property impact of common linker segments in sequence-controlled polyesters, Polym. Chem., 2019, 10, 244. Here, compounds having structure (1) are used as polyester cross-linking agents.

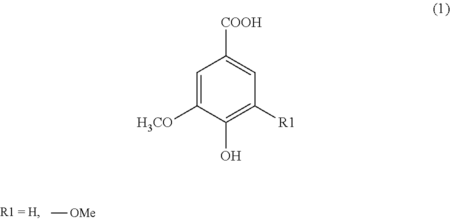

This causes a reaction with the phenol group. However, the free phenol group is essential for the stabilization effect. Thus, stabilization is not possible with the mentioned compounds.

Furthermore, structures having formula (1) are used as monomers in polymerization processes, e.g., N. Kasmi et al. Effective and facile solvent-free synthesis to novel biobased is monomers from vanillic acid: Structure thermal-property relationships of sustainable polyesters, Pol. Degr. Stab. 2020, 181, 109315 (abstract see appendix), KR 2014047208 (production of contact lenses); or U.S. Pat. No. 4,362,510 (additive to dental cement); here again, there is no longer a free phenol group after the polymerization processes.

The use of leonurine as an additive to paints and varnishes as a biocide is known (CN 104059449, CN 103881498, CN 105153852, CN 107815183, CN 107868552). The guanidine structure is decisive for the effect; however, in polymers these structures lead to incompatibilities or undesired reactions.

EP 545305 and JP H01-210948 disclose phenols as coupling agents in photographic materials; the phenol group is consumed by a reaction and formation of a bond, and thus is no longer available as an antioxidant.

However, most of the mentioned stabilizers from renewable raw materials share the limitation that although they do have a good effect as antioxidants, they also have only a limited light stabilizing effect i.e., are not especially effective UV stabilizers.

Therefore, it is the goal of this invention to make available, for plastics made from renewable raw materials, effective antioxidants which simultaneously allow a high UV protection.

### PRESENTATION OF THE INVENTION

This is accomplished by the use of derivatives of syringic acid, vanilic acid, isovanilic acid or 5-hydroxy veratric acid that are designated in claim 1 for stabilization of plastics, especially against oxidative, thermal and/or actinic degradation. Moreover, this invention relates to a plastic composition that comprises the designated derivatives of syringic acid, vanilic acid, isovanilic acid or 5-hydroxy veratric acid as stabilizers. Furthermore, this invention relates to novel derivatives of syringic acid, vanilic acid, isovanilic acid or 5-hydroxy veratric acid that are especially suitable as stabilizers of plastics, as well as a process for stabilization of a plastic composition using the derivatives of syringic acid, vanilic acid, isovanilic acid or 5-hydroxy veratric acid. Finally, this invention relates to a stabilizer composition that consists of derivatives of syringic acid, vanilic acid, isovanilic acid or 5-hydroxy veratric acid and at least one additive.

One aspect of this invention relates to the use of a compound having general formula I or II or mixtures of multiple compounds having general formula I and/or II:

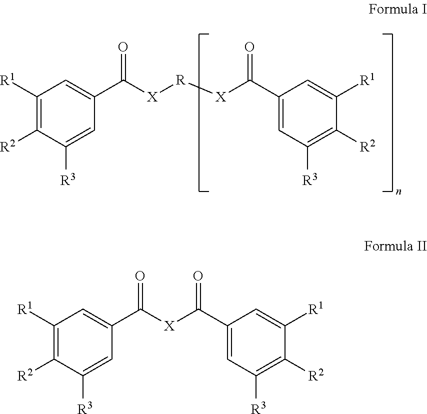

wherein
R¹, R² and R³ are selected, each independently of one another, from the group consisting of hydroxy, linear or branched alkoxy groups with 1 to 6 carbon atoms and hydrogen, with the condition that at least one of the groups R¹, R² and R³ is a hydroxyl group and at least one of the groups R¹, R² and R³ is a linear or branched alkoxy group with 1 to 6 carbon atoms, wherein every occurrence of R¹, R² and R³ is the same or different,
X upon every occurrence is the same or different and in formula I is O or NH or NR, in formula II it is O or NR, n is 0 or a whole number from 1 to 10, preferably 1 to 4, and
R is an organic group,
for stabilization of plastics, especially against oxidative, thermal and/or actinic degradation.

Thus, the mentioned compounds having general formulas I and 11 are derived from naturally occurring compounds such as
a) syringic acid

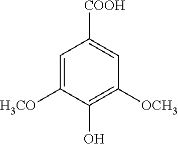 [SMILES: COc1cc(C(=O)O)cc(OC)c1O]

b) vanilic acid

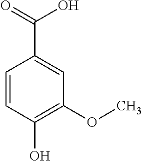

c) isovanilic acid

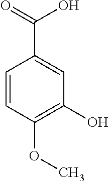

and
d) 5-hydroxy veratric acid

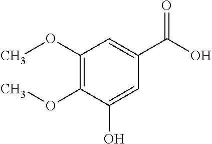

The mentioned compounds having general formulas I and II are surprisingly distinguished by high stabilizing potential in the stabilization of plastics, especially against oxidative, thermal and/or actinic degradation.

The compounds having general formula I and/or II can be prepared in a way that is known in the art.

The acids syringic acid, vanilic acid, isovanilic acid or 5-hydroxy veratric acid that are used as starting products are commercially available.

The esters (formula I: X=O, n=0 or ≠0) can be prepared, e.g., according to WO 2010/043631 or analogously to WO 98/556748. For example, aliphatic esters can be obtained by reacting an-aliphatic, alicyclic, or aromatic mono-, di-, or polyalcohol with the acid group of syringic acid, vanilic acid, isovanilic acid or 5-hydroxy veratric acid through acid catalysis e.g., in the presence of sulfuric acid or p-toluenesulfonic acid in a suitable solvent or suspending agent, such as, e.g., toluene with the water that forms being removed, e.g., by distillation.

Another possibility is a process in which a short-chain ester, such as, e.g., the methyl ester or ethyl ester of syringic acid, vanillic acid, isovanillic acid, or 5-hydroxy veratric acid, is synthesized and, in a second step, a transesterification reaction is carried out with a, e.g., longer-chain alcohol in the presence of a suitable catalyst, such as, e.g., dibutyltin butoxide, dioctyltinketonate, or tetrapropylorthotitanate. Alternatively, the esterification and/or transesterification reaction can also be enzymatic, such as described, e.g., in K. Vosmann et al. Appl. Microbiol. Biotechnol. (2008) 80:29-36.

Furthermore, a first synthesis step can be carried out in which the OH group is provided with a protective function that is removed after the esterification.

The amides of this invention can be prepared in accordance with WO 2010/043631 or in accordance with Pearl, Irwin A.; Beyer, Donald L., Reactions of vanillin and its derived compounds. XXI. Amides of vanillic and 3-ethoxy-4-hydroxybenzoic acids, Journal of the American Chemical Society (1953), 75, 2627-30.

The anhydride having formula II (X=O) can be synthesized as described in Z. Ahmadzadeh et al. Cu-MOF: an efficient heterogeneous catalyst for the synthesis of symmetric anhydrides via the C—H bond activation of aldehydes, RSC Adv., 2018, 8, 24203-24208.

A preferred embodiment provides that the group R be selected from the group consisting of linear or branched and saturated or unsaturated alkyl groups, preferably with at least 6 carbon atoms, especially preferably linear alkyl groups with 6, 8, 9, 10, 11, 12, 13, 14, 16, 18, 20, 22, 24, 26, 28 or 30 carbon atoms, 11-methyldodecan-1-yl, 3,7-dimethyl-7-octen-1-yl, (R)-3,7-dimethyloct-6-en-1-yl, 2,6-dimethyl-2,6-octadien-8-yl, cis-9-hexadecen-1-yl, cis-9-octadecen-1-yl, cis-13-docosen-1-yl, cis,cis-9,12-octadecadien-1-yl, 3,7-dimethyl-trans-2,6-octadien-1-yl, aromatic groups such as, e.g., substituted or unsubstituted phenyl or benzyl; or 2,3-dihydroxypropan-1-yl, linear or branched and saturated or unsaturated alkanediyl groups, preferably 1,2-ethanediyl, 1,3-propanediyl, 1,4-butanediyl, 1,5-pentanediyl, 1,6-hexanediyl, 1,8-octanediyl; linear or branched and saturated or unsaturated oxaalkanediyl groups, especially oxaethylenediyl- or oxapropylenediyl groups, such as, e.g., 3-oxapentane-1,5-diyl, 2,2′-(ethylenedioxy)diethanediyl, 2,4-dimethyl-3-oxapentane-1,5-diyl, 4-oxaheptane-2,6-diyl, 2-(2-hydroxypropyl)-1-propyl, 1,3-cyclohexanediyl, 1,4-cyclohexanediyl; aromatic groups, especially benzene-1,4-diyl or benzene-1,3-diyl, 2-hydroxypropane-1,3-diyl, 2-aminopropane-1,3-diyl, as well as di-, tri- or tetravalent groups derived from alditols.

It is further preferred that, always independently from one another, the group
R¹ is a linear or branched alkoxy group with 1 to 6 carbon atoms, especially methoxy,
R² is hydroxy, and
R³ is a linear or branched alkoxy group with 1 to 6 carbon atoms, especially methoxy, or hydrogen.

For example, esters (X=O, formula I: n=0) can be derived from linear or branched aliphatic alcohols, preferably with at least 6 C atoms, such as hexan-1-ol, octan-1-ol, nonan-1-ol, decan-1-ol, undecan-1-ol, lauryl alcohol, trideca.n-1-ol, myristyl alcohol, cetyl alcohol, stearyl alcohol, ceryl alcohol or myricyl alcohol, palmitoleyl alcohol, oleyl alcohol, arachidyl alcohol, behenyl alcohol, erucyl alcohol, ceryl alcohol or myricyl alcohol. ceryl alcohol or myricyl alcohol, palmitoleyl alcohol, oleyl alcohol, arachidyl alcohol, behenyl alcohol, erucyl alcohol, lignoceryl alcohol, montanyl alcohol, linoleyl alcohol, Isotridecyl alcohol, geraniol, rhodinol, citronellol or nerol, particularly preferred are the lauryl ester and the stearyl ester.

For the case that the compounds of the general formula I relate to amides (X=NH, formula I: n=0), the structures can be derived from linear or branched amines, preferably with at least 6 C atoms, such as hexane-1-amine, laurylamine or stearylamine.

Also suitable are esters of di- or polyols (formula I: X=O, n 0 or ≠0) or amides of polyamines (formula I: X=NH, n≠0).

Examples of diols are alkanediols such as 1,2-ethanediol, 1,3-propanediol, 1,4-butanediol, 1-6-hexanediol, diethyleneglycol, triethyleneglycol, dipropylene glycol, tripropyleneglycol, or higher glycol homologs. Examples of alicyclic diols are 1,4-cyclohexanediol or 1,3-cyclohexanediol.

Examples of phenols are hydroquinone or resorcin.

Just as suitable are polyols such as, e.g., glycerin and alditols, as well as the following polyalcohols:

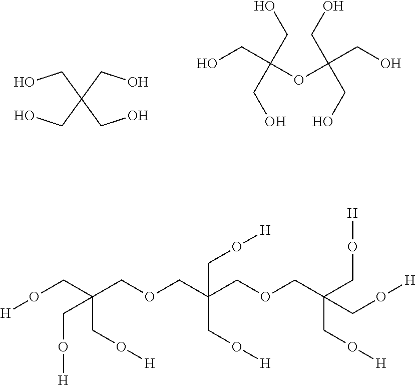 [SMILES: OCC(CO)(CO)CO ; OCC(CO)(CO)OC(CO)(CO)CO ; OCC(CO)(CO)COCC(CO)(CO)COCC(CO)(CO)CO]

Examples of diamines are 1,4-butanediamine, 1,5-pentanediamine, 1,6-hexamethylene diamine, 1,10-decanediamine, or 1,12-dodecanediamine. In the described di- or multifunctional alcohols or amines it is possible that one, multiple, or all of the OH- or NH₂ functionalities are esterified or form an amide bond with the acid groups of the above presented acids.

Especially preferred are the tetra-substituted esters of pentaerythrite.

Preferred representatives of the compounds having general formula I are selected from the group consisting of

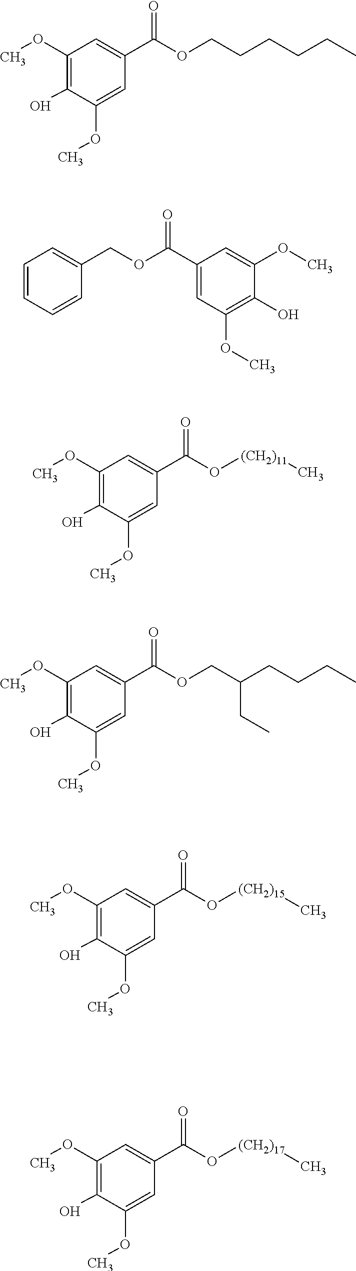

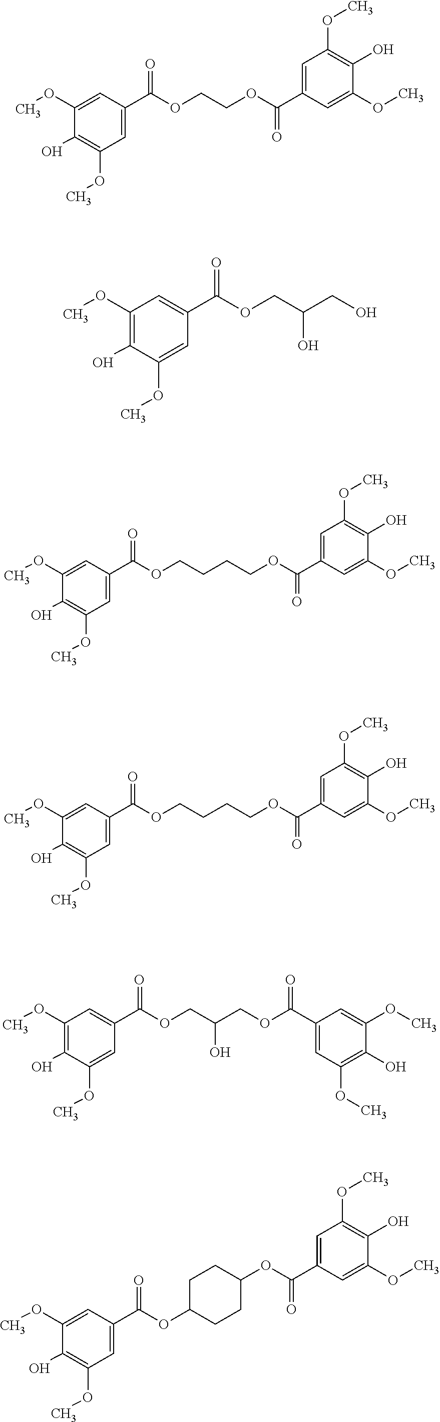

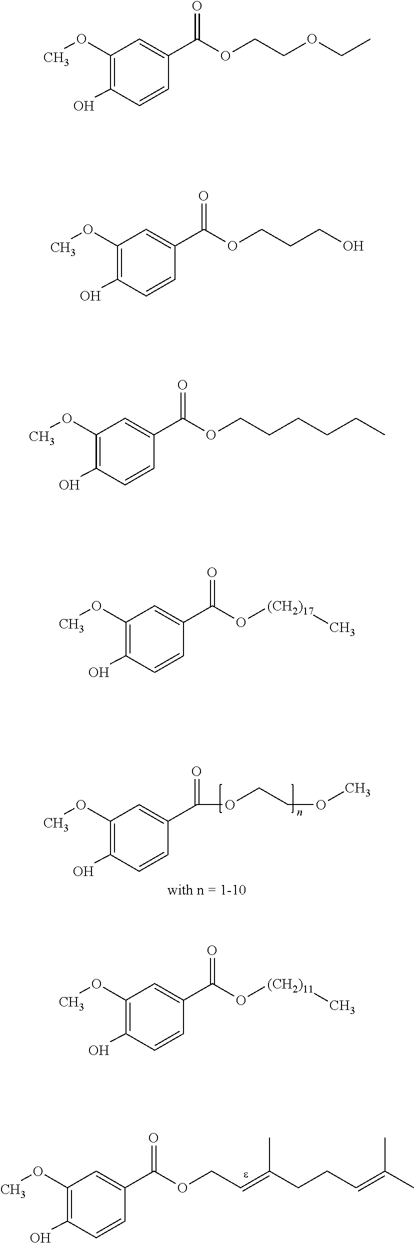

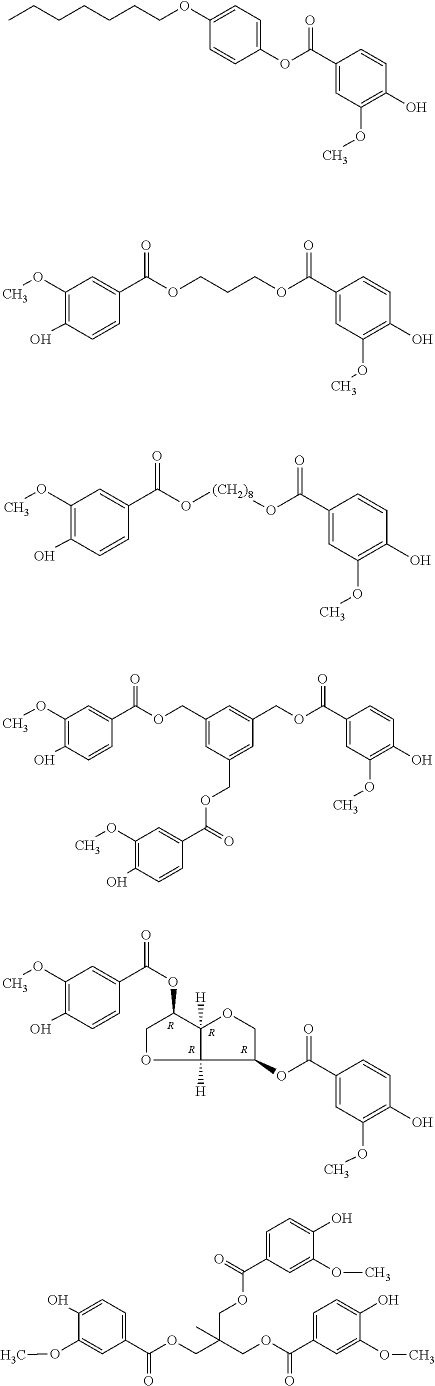

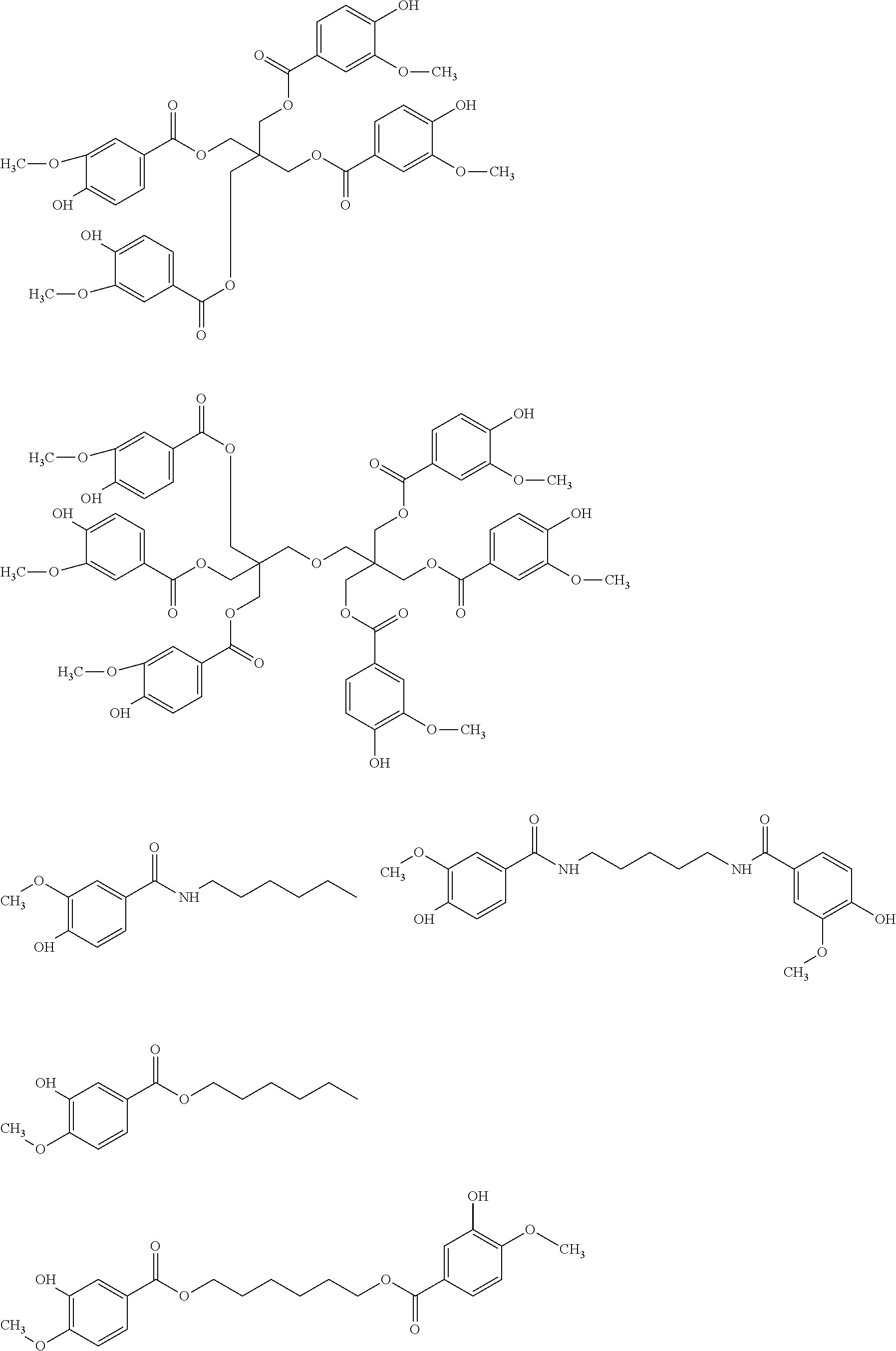

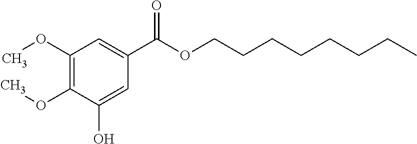

and the compound having general formula II

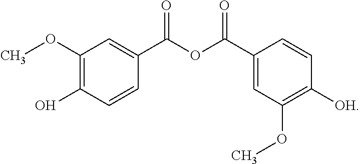

Most especially preferred are the following compounds:

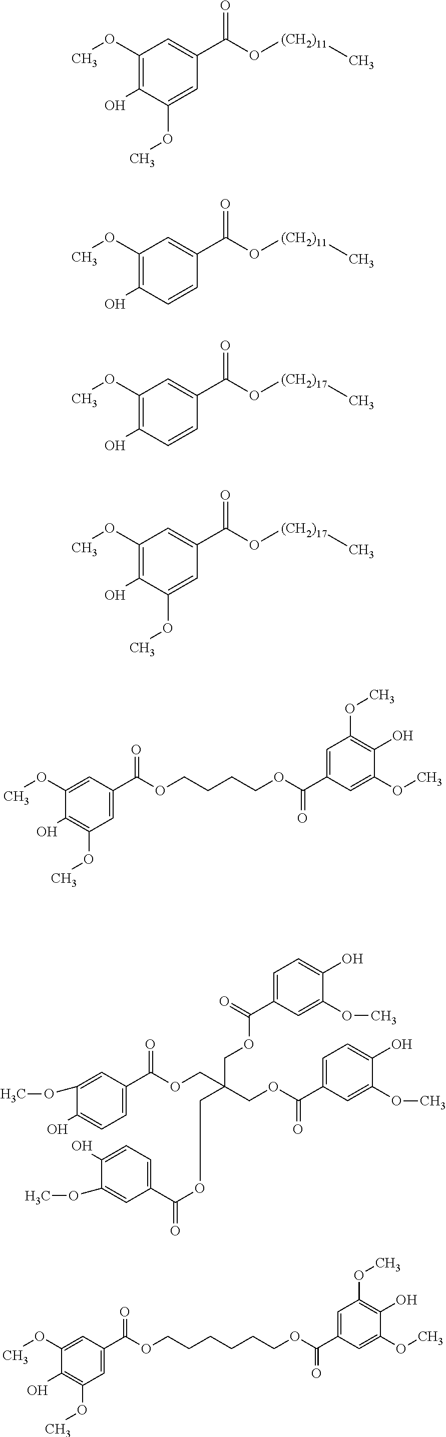

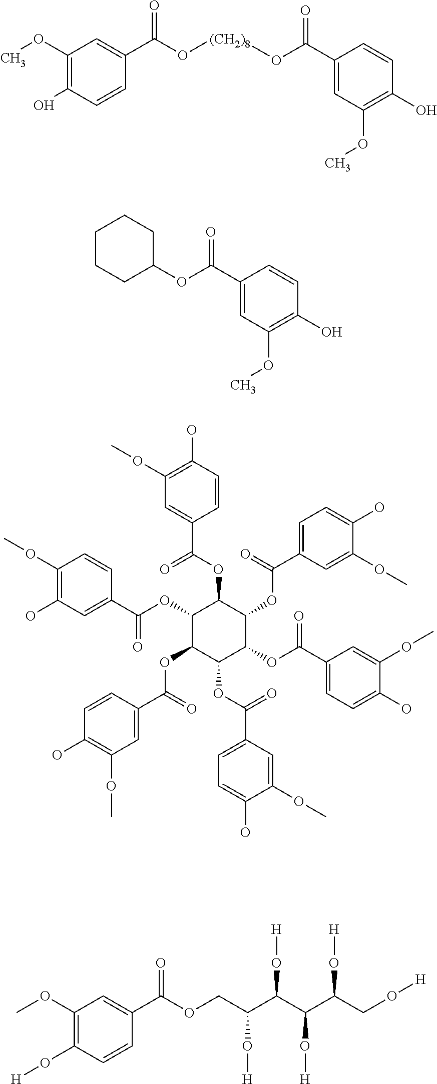

Furthermore, preferred compounds with aromatic esters or amides are, for example, the following:

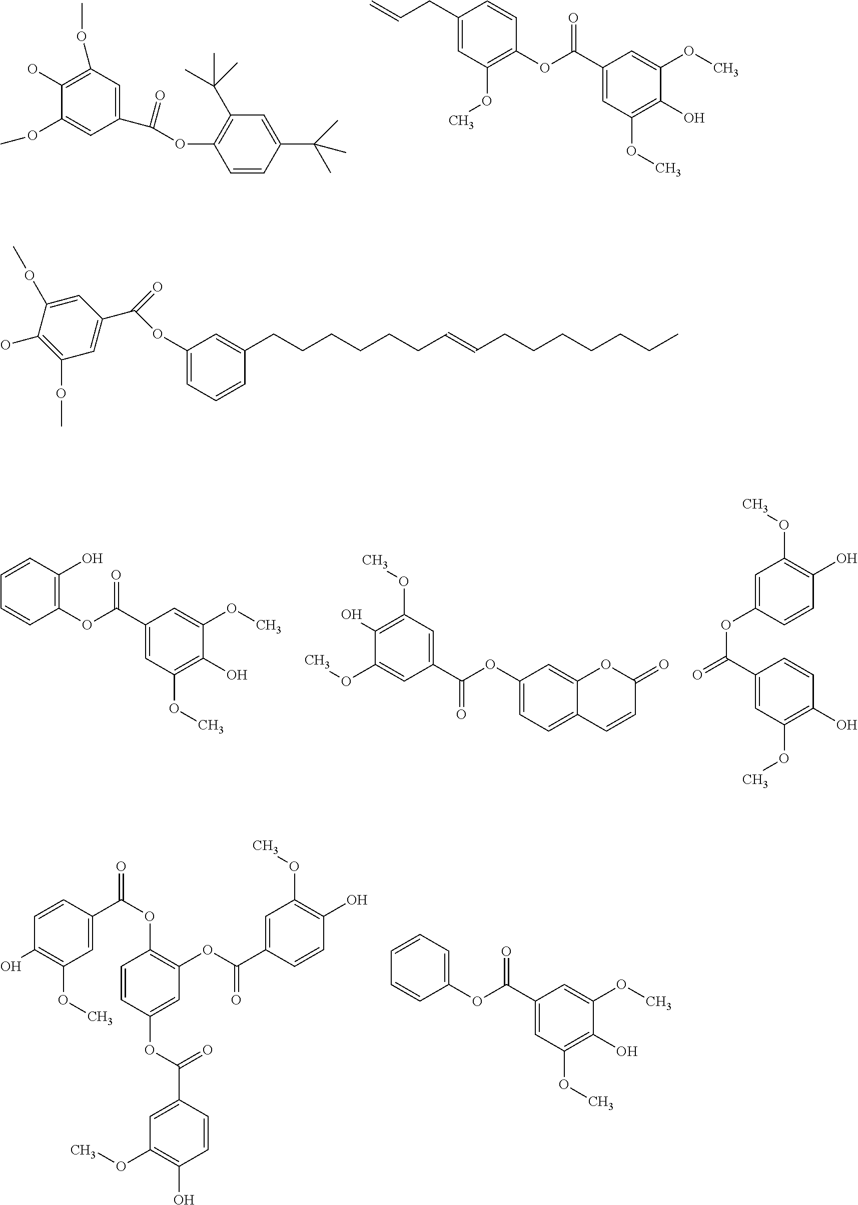

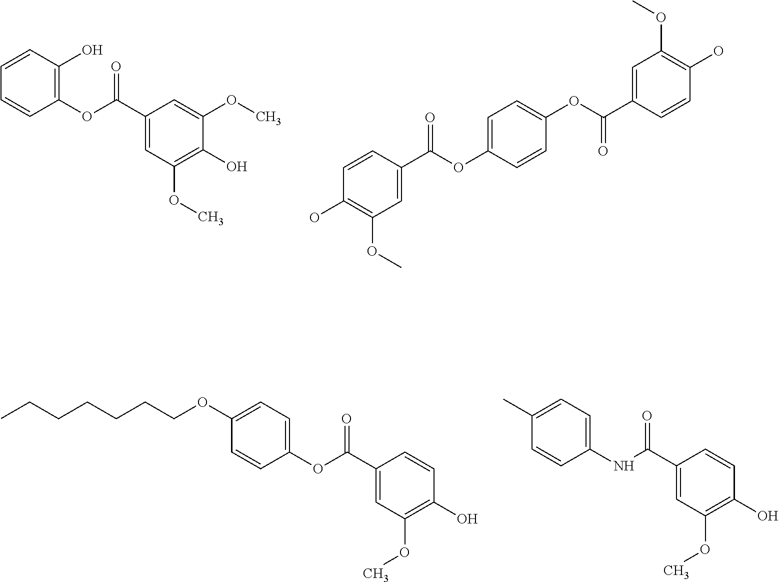 [SMILES: COc1cc(C(=O)Oc2ccccc2O)cc(OC)c1O ; COc1cc(C(=O)Oc2ccc(OC(=O)c3ccc(O)c(OC)c3)cc2)ccc1O ; CCCCCCCOc1ccc(OC(=O)c2ccc(O)c(OC)c2)cc1 ; COc1cc(C(=O)Nc2ccc(C)cc2)ccc1O]

Another preferred embodiment provides that the compound having general formula I or II, or in the case of a mixture of multiple compounds having general formula I and/or II, the sum of all compounds having general formula I and/or II is/are contained in the plastic in a weight percentage from 0.01 to 10.00 weight %, preferably from 0.02 to 5.00 weight %, especially preferably from 0.05 to 3.00 weight %.

The plastics to be stabilized are, e.g., thermoplastic, thermoset, or elastomeric polymers.

The compounds having general formula I and/or 11 are especially suitable for stabilizing plastics, the plastic being selected from the group consisting of
a) polymers from olefins or diolefins, such as, e.g., polyethylene (LDPE, LLDPE, VLDPE, ULDPE, MDPE, HDPE, UHMWPE), metallocene-PE (m-PE), polypropylene, polyisobutylene, poly-4-methyl-pentene-1, polybutadiene, polyisoprene, such as, e.g., also natural rubber (NR), poly(cyclooctene), polyalkylene-carbon monoxide copolymers, and copolymers in the form of statistical or block structures, such as, e.g., polypropylene-polyethylene (EP), EPM or EPDM with, e.g., 5-ethylidene-2-norbornene as comonomer, ethylene-vinyl acetate (EVA), ethylene acrylates, such as, e.g., ethylene-butylacrylate, ethylene-acrylic acid and salts thereof (ionomers), as well as terpolymers such as, e.g., ethylen-acrylic acid(meth)acrylate, graft polymers such as, e.g., polypropylene graft-malic acid anhydride, as well as blends such as, e.g., LDPE/LLDPE or also long-chain polypropylene copolymers that are prepared with alpha olefins as comonomers, such as, e.g., with 1-butene, 1-hexene, 1-octene, or 1-octadecene.
b) polystyrene, polymethylstyrene, poly-alpha-methylstyrene, polyvinylnaphthalene, polyvinylbiphenyl, polyvinyl toluene, styrene-butadiene (SB), styrene-butadiene-styrene (SBS), styrene-ethylene-butylen-e styrene (SEBS), styrene-ethylene-propylene styrene, styrene-isoprene, styrene-isoprene styrene (SIS), acrylonitrile butadiene styrene (ABS), styrene acrylonitrile (SAN), styrene acrylonitrile acrylate (ASA), styrene ethylene, styrene maleic acid anhydride polymers including corresponding graft polymers, such as, e.g., styrene to butadiene, maleic acid anhydride to SBS or SEBS, and graft copolymers from methyl methacrylate, styrene butadiene and ABS (MABS), as well as hydrogenated polystyrene derivatives such as, e.g., polyvinylcyclohexane,
c) halogen-containing polymers such as, e.g., polyvinyl chloride (PVC), polychloroprene and polyvinyldichloride (PVDC), copolymers of vinyl chloride and vinylidene chloride or of vinyl chloride and vinyl acetate, chlorinated polyethylene, polyvinylidene fluoride, epichlorohydrin-homopolymers and copolymers especially with ethylene oxide (ECO),
d) polymers of unsaturated esters such as, e.g., polyacrylates and polymethacrylate such as polymethylmethacrylate (PMMA), polybutylacrylate, polylaurylacrylate, polystearylacrylate, polyglycidylacrylate, polyglycidylmethacrylate, polyacrylonitrile, polyacrylamides, copolymers such as, e.g., polyacrylonitrile-poly(alkyl acrylate,
e) polymers of unsaturated alcohols and derivatives such as, e.g., polyvinyl alcohol, polyvinyl acetate, polyvinyl butyral, polyallyl phthalate, polyallyl melamine,
f) polyacetalenes, such as, e.g., polyoxymethylene (POM) or copolymers with, e.g., butanal,
g) polyphenylene oxides and blends with polystyrene or polyamides,
h) polymers of cyclic ethers such as, e.g., polyethylene glycol, polypropylene glycol, poly(ethylene oxide), poly(propylene oxide), polytetrahydrofuran,
i) polyurethanes of hydroxy-terminated polyethers or polyesters and aromatic or aliphatic isocyanates such as, e.g., 2,4- or 2,6 toluylene diisocyanate or methylene diphenyl diisocyanate, especially also linear polyurethane (TPU), polycarbamides,
j) polyamides such as, e.g., polyamide 6, 6.6, 6.10, 4.6, 4.10, 6.12, 10.10, 10.12, 12.12, polyamide 11, polyamide 12, as well as (partially) aromatic polyamides such as, e.g., polyphthalamides, e.g., prepared from terephthalic acid and/or isophthalic acid and aliphatic diamines such as, e.g., hexamethylene diamine or m-xylylene diamine or from aliphatic dicarboxylic acids such as, e.g., adipic acid or sebacic acid and aromatic diamines such as, e.g., 1,4- or 1,3-diaminobenzene, blends of different polyamides such as, e.g., PA 6 and PA 6.6 or blends of polyamides and polyolefins such as, e.g., PA/PP,
k) polyimides, polyamide imides, polyether imides, polyester imides, poly(ether)ketones, polysulfones, polyether sulfones, polyaryl sulfones, poly(phenylene sulfides), poly(benzimidazoles), polyhydantoins,
l) polyesters of aliphatic or aromatic dicarboxylic acides and diols or of hydroxy-carboxylic acids such as, e.g., polyethylene terephthalate (PET), polybutylene terephthalate (PBT), polypropylene terephthalate (PTI), polyethylene naphthalate (PEN), poly-1,4-dimethylolcyclohexane terephthalate, poly(hydroxybenzoate), polyhydroxynaphthalate, polylactic acid (PLA), polyhydroxybutyrate (PHB), polyhydroxyvalerate (PHV), poly(ethylene succinate), poly(tetramethylene succinate), polycaprolacton,
m) polycarbonates, polyester carbonates and blends, such as, e.g., PC/ABS, PC/PBT, PC/PET/PBT, PC/PA,
n) cellulose derivatives such as, e.g., cellulose nitrate, cellulose acetate, cellulose propionate, cellulose butyrate,
o) epoxy resins consisting of di- or polyfunctional epoxides in combination with, e.g., hardeners based on amines, anhydrides, dicyandiamide, mercaptans, Isocyanates or catalytically active hardeners,
p) phenolic resins such as, e.g., phenol formaldehyde resins, urea-formaldehyde resins, melamine formaldehyde resins,
q) unsaturated polyester resins made of unsaturated dicarboxylic acids and diols with vinyl compounds, e.g., styrene, alkyd resins,
r) silicones, e.g., based on dimethyl siloxanes, methyl phenyl siloxanes or diphenyl siloxanes, e.g., vinyl group-terminated ones,
s) as well as mixtures, combinations or blends of two or more of the previously mentioned polymers.

If the polymers indicated in a) through r) are copolymers, they can be in the form of statistical (“random”), block- or “tapered” structures. Furthermore, the mentioned polymers can be in the form of linear, branched, star-shaped, or hyperbranched structures.

If the polymers indicated in a) through r) are stereoregular polymers, they can be in isotactic, stereotactic, but also atactic forms, or in the form of stereo block copolymers.

The polyolefins mentioned under a) can possibly also be cross-linked, e.g., cross-linked polyethylene, which is then designated as X-PE.

Furthermore, these compounds can be used for stabilization of rubbers and elastomers. These can be natural rubber (NR) or synthetic rubber materials.

Suitable synthetic rubber materials consist especially of butadiene (BR), styrene-butadiene (SBR), chloroprene (CR), isoprene (IR), isobutylene-isoprene, acrylonitrile-butadiene (NBR or, in hydrogenated form HNBR). Other suitable rubbers and elastomers are ethylene propylene diene terpolymers (EPDM) and ethylene-propylene copolymers (EPM), polyester-urethane (AU), polyether-urethane (EU), and silicones (MQ).

In addition to virgin material, the plastics can be recycled plastics, e.g., from industrial collections, such as, e.g., production waste, or plastics from household or recycling collections.

Furthermore, polymers that are especially preferred are those from renewable raw materials such as, e.g., polylactic acid (PLA), polyhydroxybutanoic acid (PHB), poly(hydroxyvaleric acid) (PHV), polybutylene succinate (PBS), polybutylene succinate-co-adipate (PBSA), poly(ethylene succinate), poly(tetramethylene succinate).

Furthermore, the polymers indicated under a) through r) can have both amorphous and also (partially) crystalline morphologies.

The plastics that are preferred are thermoplastic plastics, especially preferred polymers are those from olefins or diolefins and polystyrene polymers.

Another preferred group of polymers are the polyamides and polyesters.

Another preferred embodiment provides that at least one other additive, selected from the group consisting of primary and/or secondary antioxidants, especially primary and and/or secondary antioxidants selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents and antifogging agents is contained in and/or added to the plastic when it is used.
wherein the at least one additive is preferably selected from the group consisting of phosphites, phosphonites, sulfites, polyols, acid scavengers, hindered amines as well as mixtures and combination thereof.

Primary antioxidants act as H-donors and as free radical scavengers and thus interrupt the radical autooxidation process in polymers. Suitable primary antioxidants are phenolic antioxidants, (partially) aromatic amines, hydroxylamines, and lactones.

Suitable synthetic phenolic antioxidants are, for example:
alkylated monophenols such as, e.g., 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-a-methylcyclohexyl)-4,6-dimethylphenol, 2,6-dioctadecyl-4-methylphenol, 2,4,6-tricyclohexylphenol, 2,6-di-tert-butyl-4-methoxymethylphenol, linear or branched nonylphenols such as, e.g., 2,6-dinonyl-4-methylphenol, 2,4-dimethyl-6-(1′-methylundec-1′-yl)phenol, 2,4-dimethyl-6-1′-methylheptadec-1′-yl)phenol, 2,4-dimethyl-6-(1′-methyltridec-1′-yl)phenol and mixtures thereof; alkylthiomethylphenols such as, e.g., 2,4-dioctylthiomethyl-6-tert-butylphenol, 2,4-dioctylthiomethyl-6-methylphenol, 2,4-dioctylthiomethyl-6-ethylphenol, 2,6-didodecylthiomethyl-4-nonylphenol;
hydroquinones and alkylated hydroquinones such as, e.g., 2,6-di-tert-butyl-4-methyoxyphenol, 2,5-di-tert-hydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenylstearate, bis(3,5-di-tert-butyl-4-hydroxylphenyl)adipate;
tocopherols such as, e.g., α-, β-, γ-, δ-tocopherol and mixtures thereof (vitamin E); hydroxylated thiodiphenylethers such as, e.g., 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide;
alkylidenebisphenole such as, e.g., 2,2′methylenebis(6-tert-butyl-4-methylphenol), 2,2′-methylenebis(6-tert-butyl-4-ethylphenol), 2,2′-methylenebis[4-methyl-6-(a-methylcyclohexyl)phenol], 2,2′-methylenebis(4-methyl-6-cyclhexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-a-methylbenzyl)-4-nonylphenol], 2,2′-methylenebis[6-(α,α-dimethylbenzyl)-4-nonylphenol], 4,4′-methylenebis(2,6-di-tert-butylphenol, 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethyleneglycol-bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis-3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane
O-, N-, and S-benzyl compounds such as, e.g., 3,5,3′,5′-tetra-tert-butyl-4,4′-dihydroxydibenzylether, octadecyl-4-hydroxy-3,5-dimethylbenzylmercaptoacetate, tridecyl-4-hydroxy-3,5-di-tert-butylbenzyl-mercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate;
hydroxybenzylated malonates such as, e.g., dioctadecyl-2,2-bis(3,5-di-tert-butyl-2-hydroxybenzyl)-malonate, dioctadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis-(3,5-di-tert-butyl-4-hydroxybenzyl)malonate; aromatic hydroxybenzyl compounds such as, e.g., 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol;
triazine compounds such as, e.g., 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxphenylethyl)-1,3,5-triazine, 1,3,5-tris(3,5-di-tert-butyl-4-hydroyphenylpropionyl)hexahydro-1,3,5-triazine, 1,3,5-tris(3,5-dicyclohexyl-4-hydroxybenzyl)isocyanurate;
benzylphosphonates such as, e.g., dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, di-octadecyl-5-tert-butyl-4-hydroxy-3-methylbenzylphosphonate, the calcium salt of the monoethylester of 3,5-di-tert-butyl-4-hydroxybenzylphosphonic acid;
Acylaminophenols such as, e.g., 4-hydroxylauranilide, 4-hydroxystearanilide, octyl-N-(3,5-di-tert-butyl-4-hydroxyphenyl)carbamate;
Esters of β-(3,5-di-tert-butyl-4-hydroxyphenyl)propionic acid with monohydric or polyhydric alcohols, e.g., methanol, ethanol, n-octanol, i-octanol, octadecanol, 1,6-hexanediol, 1,9-nonanediol, ethyleneglycol, 1,2-propanediol, neopentylglycol, thiodiethylenglycol, diethyleneglycol, triethyleneglycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
Esters of β-(5-tert-butyl-4-hydroxy-3-methylphenyl)propionic acid with monohydric or polyhydric alcohols, e.g., methanol, ethanol, n-octanol, i-octanol, octadecanol, 1,6-hexanediol, 1,9-nonanediol, ethyleneglycol, 1,2-propanediol, neopentylglycol, thiodiethyleneglycol, diethyleneglycol, triethyleneglycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, 3,9-bis[2-{3-(3-tert-butyl-4-hydroxy-5-methylphenyl)propionyloxy}-1,1-dimethylethyl]-2,4,8,10-tetraoxaspiro[5.5]undecane;
Esters of β-(3,5-dicyclohexyl-4-hydroxyphenyl)proprionic acid with monohydric or polyhydric alcohols, e.g., methanol, ethanol, n-octanol, i-octanol, octadecanol, 1,6-hexandiol, 1,9-nonandiol, ethyleneglycol, 1,2-propanediol, neopentylglycol, thiodiethylenglycol, diethyleneglycol, triethyleneglycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;

Esters of β-(3,5-di-tert-butyl-4-hydroxyphenyl)acetic acid with monohydric or polyhydric alcohols, e.g., methanol, ethanol, octanol, octadecanol, 1,6-hexanediol, 1,9-nonanediol, ethyleneglycol, 1,2-propanediol, neopentylglycol, thiodiethyleneglycol, diethyleneglycol, triethyleneglycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;

Amides of β-(3,5-di-tert-butyl-4-hydroxyphenyl)propionic acid such as, e.g., N,N′-bis(3,5-di-tert-butyl-4-hydroxyphenylpropionyl)hexamethylendiamide, N,N′-bis(3,5-di-tert-butyl-4-hydroxyphenylpropionyl)-hexamethylendiamide, N,N′-bis(3,5-di-tert-butyl-4-hydroxyphenylpropionyl)hexamethylenediamide, N,N′-bis-(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, marketed by Uniroyal);

### Vitamin C

Especially preferred phenolic antioxidants are:

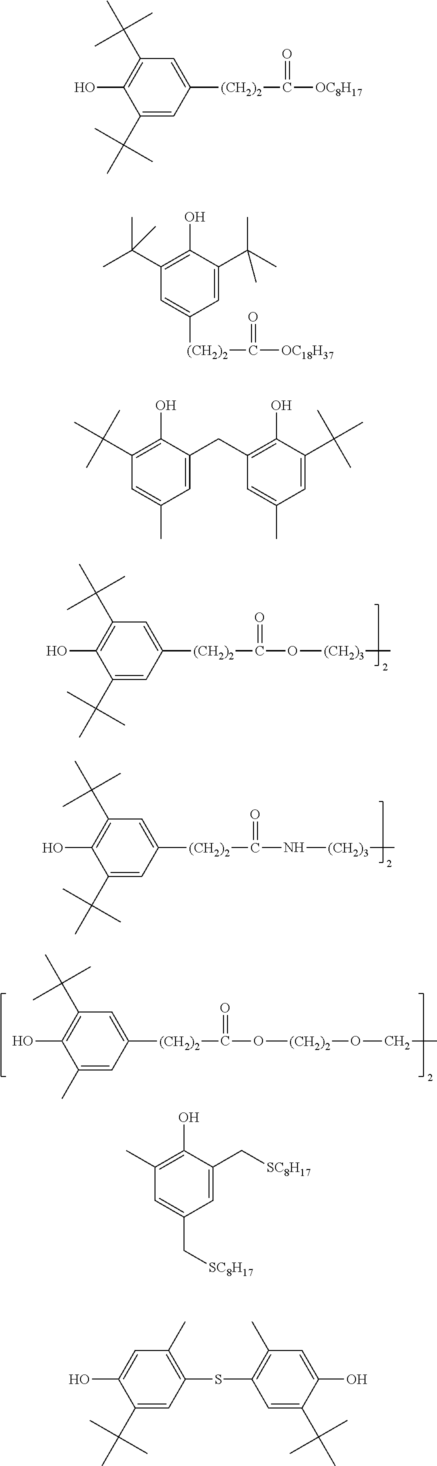

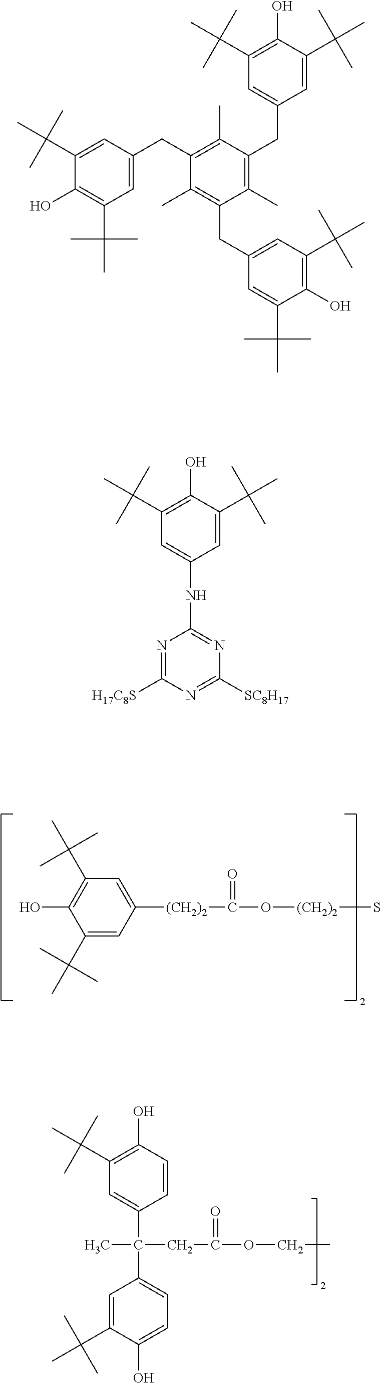

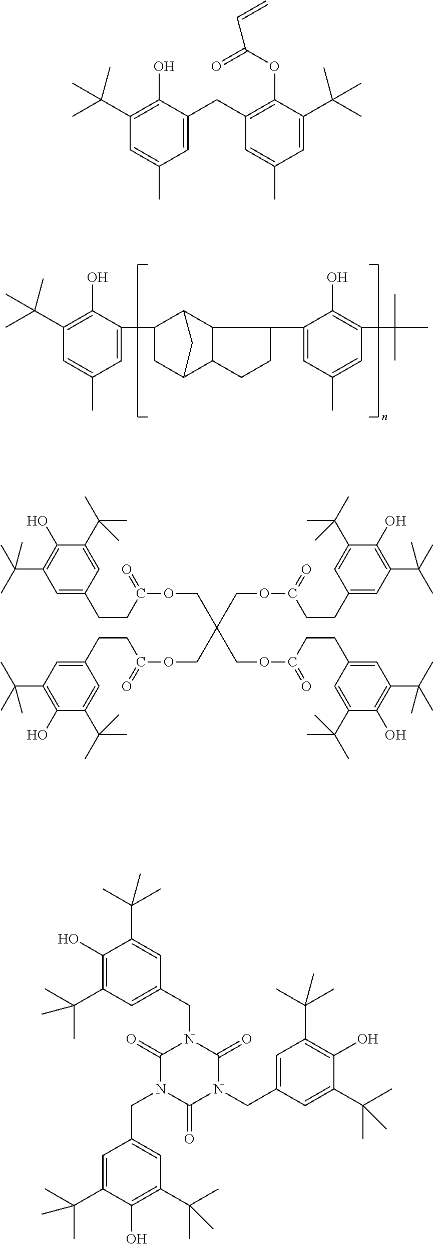

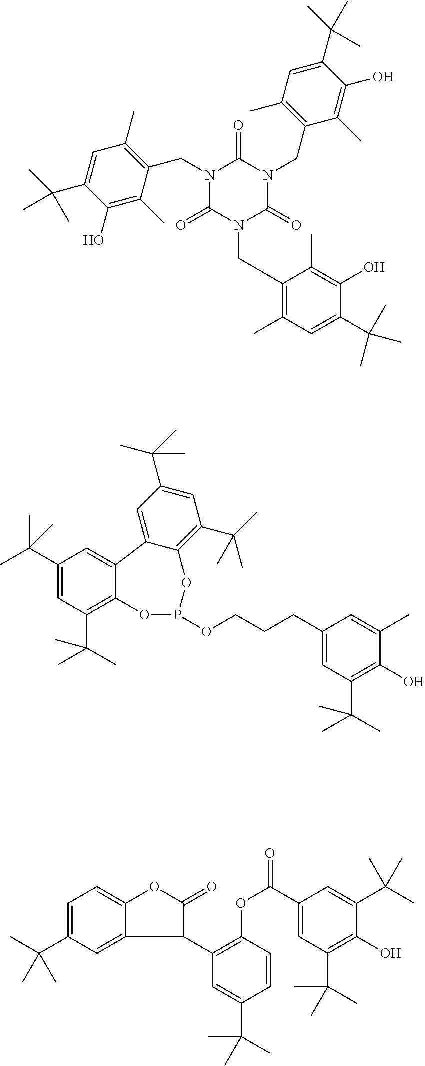

Other especially preferred phenolic antioxidants are based on renewable raw materials such as, e.g., tocopherols (vitamin E), tocotrienole, tocomonoenols, carotenoids, hydroxytyrosol, flavonols such as, e.g., chrysin, quercitin, hesperidin, neohesperidin, naringin, morin, kaempferol, fisetin, anthocyans such as, e.g., Delphinidin and malvidin, curcumin, carnosolic acid, carnosol, rosmarinic acid, resveratrol, and tannins.

Examples of suitable antioxidants are:
N,N′-di-isopropyl-p-phenylenediamine, N,N′-di-sec-butyl-p-phenylenediamine, N,N′-bis(1,4-dimethyl-pentyl)-p-phenylenediamine, N,N′-bis(1-ethyl-3-methylpentyl)-p-phenylenediamine, N,N′-bis(1-methyl-heptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethyl-butyl)-N′-phenyl-p-phenylenediamine, N-(1-methylheptyl)-N′-phenyl-p-phenylenediamine, N-cyclohexyl-N′-phenyl-p-phenylenediamine, 4-(p-toluolsulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-4-tert-octylphenyl)-1-naphthylamine, N-phenyl-2-naphthylamine, octylated diphenylamine, e.g., p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetra-methyl-4,4′-diaminodiphenylmethane, 1,2-bis[(2-methyl-phenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazines, N-allylphenothiazine, N,N,N′,N′-tetraphenyl-1,4-diaminobut-2-ene, and mixtures or combinations thereof.

Preferred amine antioxidants are: N,N′-di-isopropyl-p-phenylenediamine, N,N′-di-secbutyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine.

Especially preferred amine antioxidants are those having the structures:

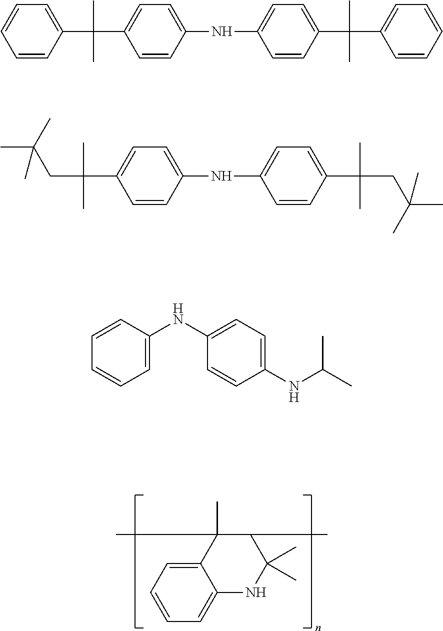

Examples of preferred hydroxylamines or N-oxides (nitrons) are, N,N-dialkylhydroxylamines, N,N-dibenzylhydroxylamine, N,N-dilaurylhydroxylamine, N,N-distearylhydroxylamine, N-benzyl-α-phenylnitron, N-octadecyl-α-hexadecylnitron, as well as Genox™ EP (SI Group) having the formula:

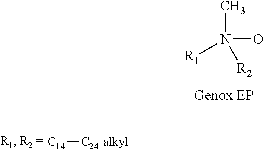

Examples of suitable lactones are benzofuranones and indolinones are 3-(4-(2-acetoxyethoxy)phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-(2-hydroxyethoxy]phenyl)benzofuran-2-one), 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-dimethylphenyl)-5,7-di-tert-butyl-benzofuran-2-one, as well as lactones that additionally contain phosphite groups, such as, e.g.,

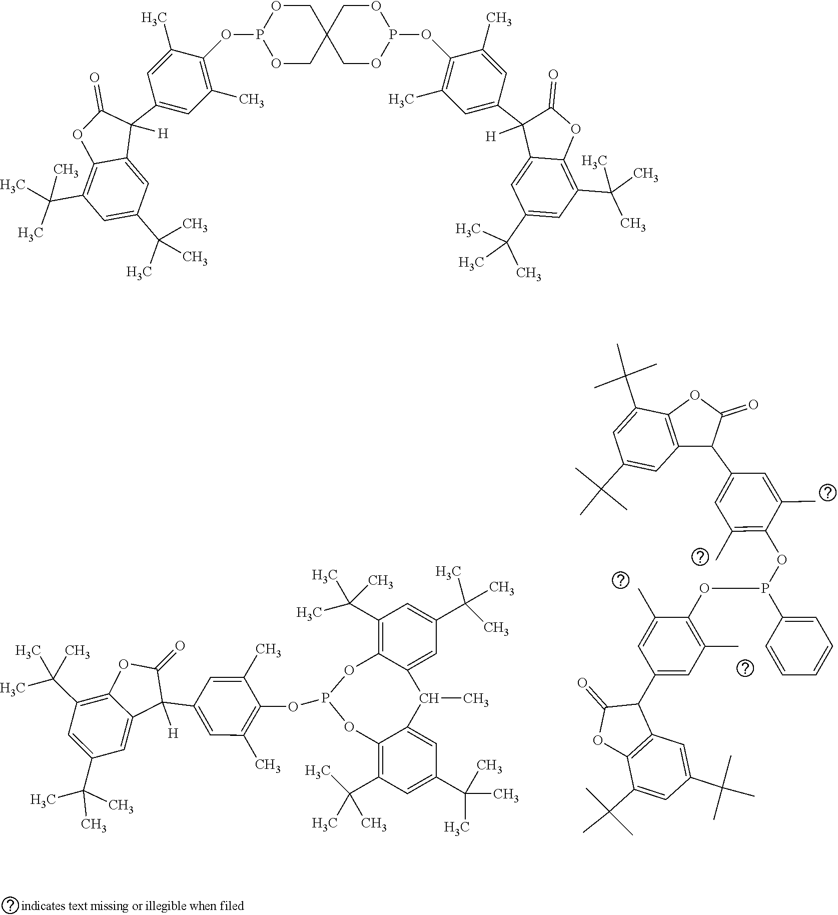

An especially preferred lactone has the following structure:

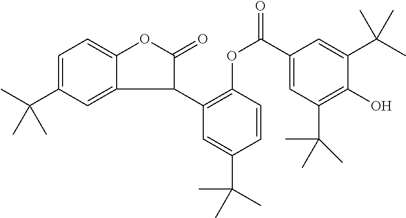 [SMILES: CC(C)(C)c1ccc(OC(=O)c2cc(C(C)(C)C)c(O)c(C(C)(C)C)c2)c(C2C(=O)Oc3ccc(C(C)(C)C)cc32)c1]

Another suitable group of antioxidants is isoindolo[2,1-A]quinazolines, such as, e.g.,

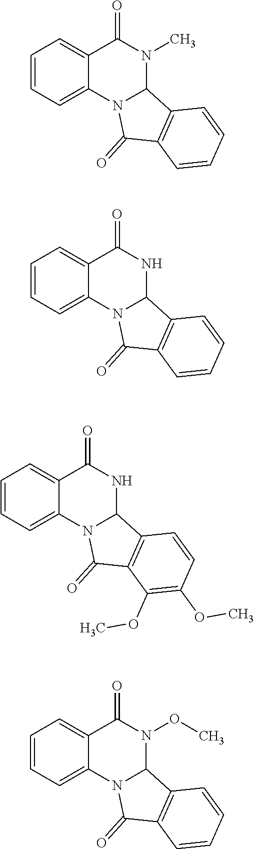 [SMILES: CN1C(=O)c2ccccc2N2C(=O)c3ccccc3C12 ; O=C1NC2c3ccccc3C(=O)N2c2ccccc21 ; COc1ccc2c(c1OC)C(=O)N1c3ccccc3C(=O)NC21 ; CON1C(=O)c2ccccc2N2C(=O)c3ccccc3C12]

Secondary antioxidants stabilize plastics primarily by decomposing hydroperoxide.

Suitable secondary antioxidants are especially phosphites or phosphonites such as, e.g., triphenylphosphite, diphenylalkylphosphites, phenyldialkylphosphites, tri(nonylphenyl)phosphite, trilaurylphosphites, trioctadecylphosphite, distearylpentaerythritoldiphosphite, tris-(2,4-di-tert-butylphenyl)phosphite, diisodecylpentaerythritoldiphosphite, bis(2,4-di-tert-butylphenyl)pentaerythritoldiphosphite, bis(2,4-di-cumylphenyl)pentaerythritoldiphosphite, bis(2,6-di-tert-butyl-4-methylphenyl)pentaerythritoldiphosphite, diisodecyloxypentaerythritoldiphosphite, bis(2,4-di-tert-butyl-6 methylphenyl)pentaerythritoldiphosphite, bis(2,4,6-tris(tert-butylphenyl)pentaerythritoldiphosphite, tristearylsorbitoltriphosphite, tetrakis(2,4-di-tert-butylphenyl)-4,4′-biphenylene-diphosphonite, 6-isooctyloxy-2,4,8,10-tetra-tert-butyl-12H-dibenz[d,g]-1,3,2-dioxaphosphocine, bis(2,4-di-tert-butyl-6-methylphenyl)methylphosphite, bis(2,4-di-tert-butyl-6-methylphenyl)-ethylphosphite, 6-fluoro-2,4,8,10-tetra-tert-butyl-12-methyl-dibenz[d,g]-1,3,2-dioxaphosphocine, 2,2′2″-nitrilo[triethyl-tris(3,3″,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl))phosphite, 5-butyl-5-ethyl-2-(2,4,6-tri-tert-butylphenoxy)-1,2-dioxaphosphirane.

Especially preferred phosphites are:

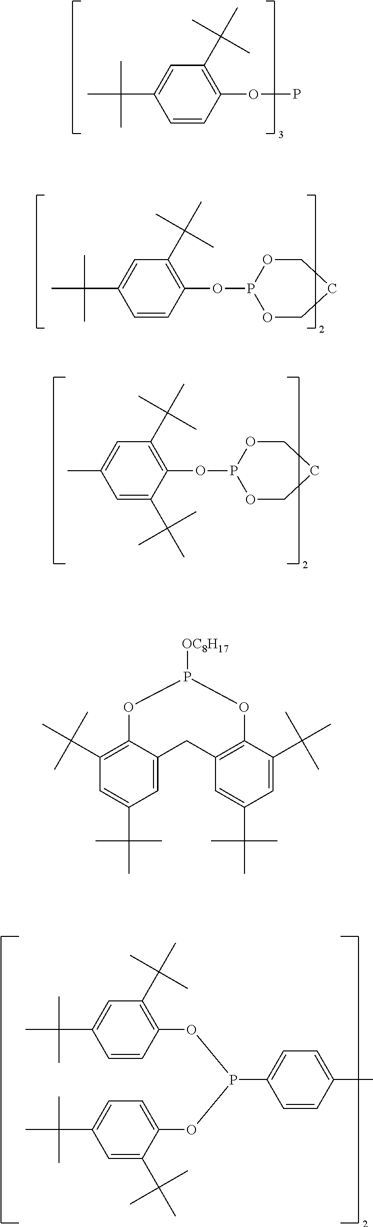

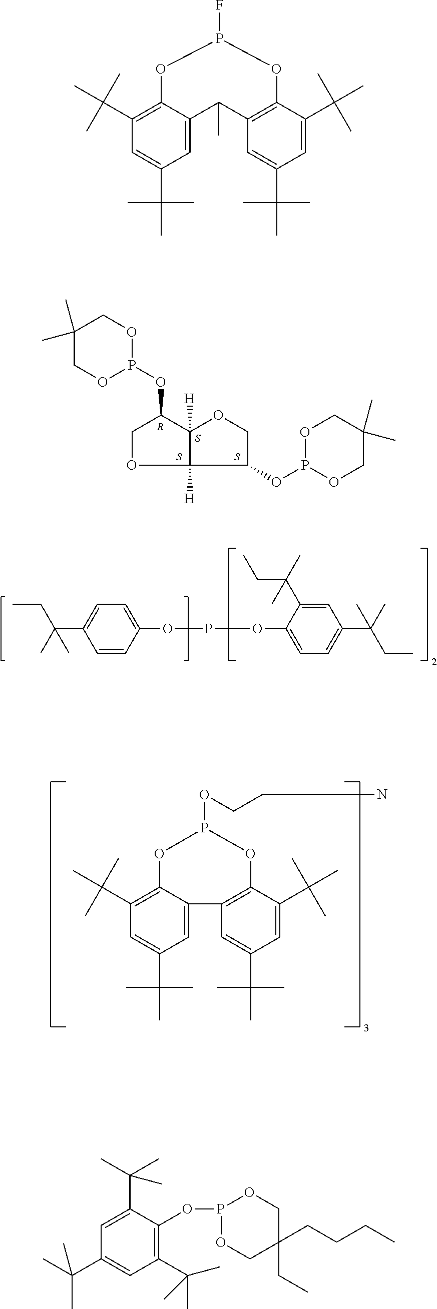

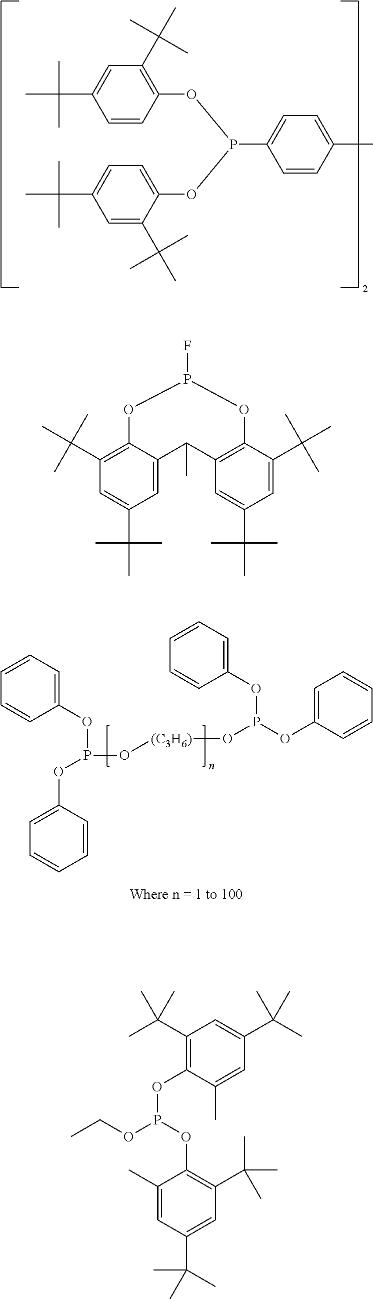

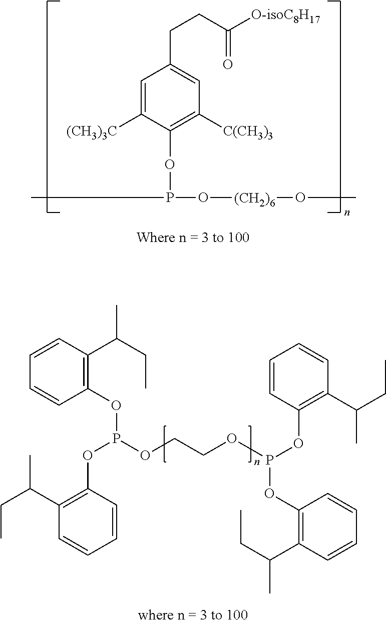

A preferred phosphonite is

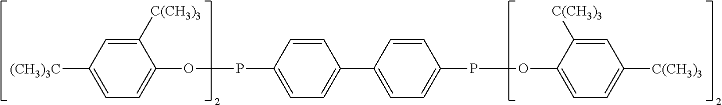

Still other suitable secondary antioxidants are sulfur compounds such as, for example, for example distearylthiodipropionate, dilaurylthiodipropionate; ditridecyldithiopropionate, ditetradecylthiodipropionate, 3-(dodecylthio)-1,1′-[2,2-bis[[3-(dodecylthio)-1-oxopropoxy]methyl]-1,3-propandiyl]propanoic acid ester. The following structures are preferred:

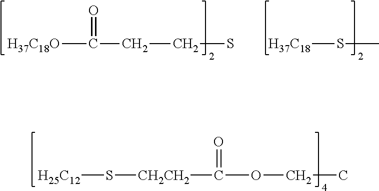

Other suitable secondary antioxidants are sulfites:

Preferred sulfite antioxidants are inorganic sulfites, disulfites, or thiosulfates of a monovalent, divalent, trivalent, or tetravalent metal, the preferably being an alkali metal, an alkaline earth metal, aluminum and/or zinc, and the inorganic sulfite being used especially in its anhydrous form.

Suitable salts are especially sodium sulfite, potassium sulfite, lithium sulfite, calcium sulfite, magnesium sulfite, aluminum sulfite, or zinc sulfite. Others that are suitable are thiosulfates such as, e.g., sodium thiosulfate.

Suitable fillers and reinforcing substances are, for example, synthetic or natural materials such as, e.g., calcium carbonate, silicates, glass fibers, glass beads (solid or hollow), talc, mica, kaolin, barium sulfate, metal oxides and metall hydroxides, soot, graphite, carbon nanotubes, graphene, wood dust or fibers of natural products such as, e.g., cellulose or synthetic fibers. Other suitable fillers are hydrotalcites or zeolites or layer silicates such as, e.g., montmorillonite, bentonite, beidellite, mica, hectorite, saponite, vermiculite, hydrobiotite, magadiite, illite, kaolinite, wollastonite, attapulgite.

Suitable acid scavengers (“antiacids”) are salts of monovalent, divalent, trivalent, or tetravalent metals, preferably alkali metals, alkaline earth metals, aluminum or zinc, especially those formed with fatty acids such as, e.g., calcium stearate, magnesium stearate; zinc stearate, aluminum stearate, calcium laurate, calcium behenate, calcium lactate, calciumstearoyl-2-lactate. Other classes of suitable acid scavengers are hydrotalcites, especially synthetic hydrotalcites based on aluminum, magnesium, and zinc, hydrocalumites, zeolites, alkaline earth oxides, especially calcium oxide and magnesium oxide as well as zinc oxide, alkaline earth carbonates, especially calcium carbonate, magnesium carbonate and dolomite, as well as hydroxides, especially brucite (magnesium s hydroxide).

Other suitable costabilizers are polyols, especially alditols or cyclitols. Polyols are, e.g., pentaerythrite, dipentaerythrite, tripentaerythrite, short-chain polyether polyols or polyester polyols, as well as hyperbranched polymers/oligomers or dendrimers having alcohol groups e.g.

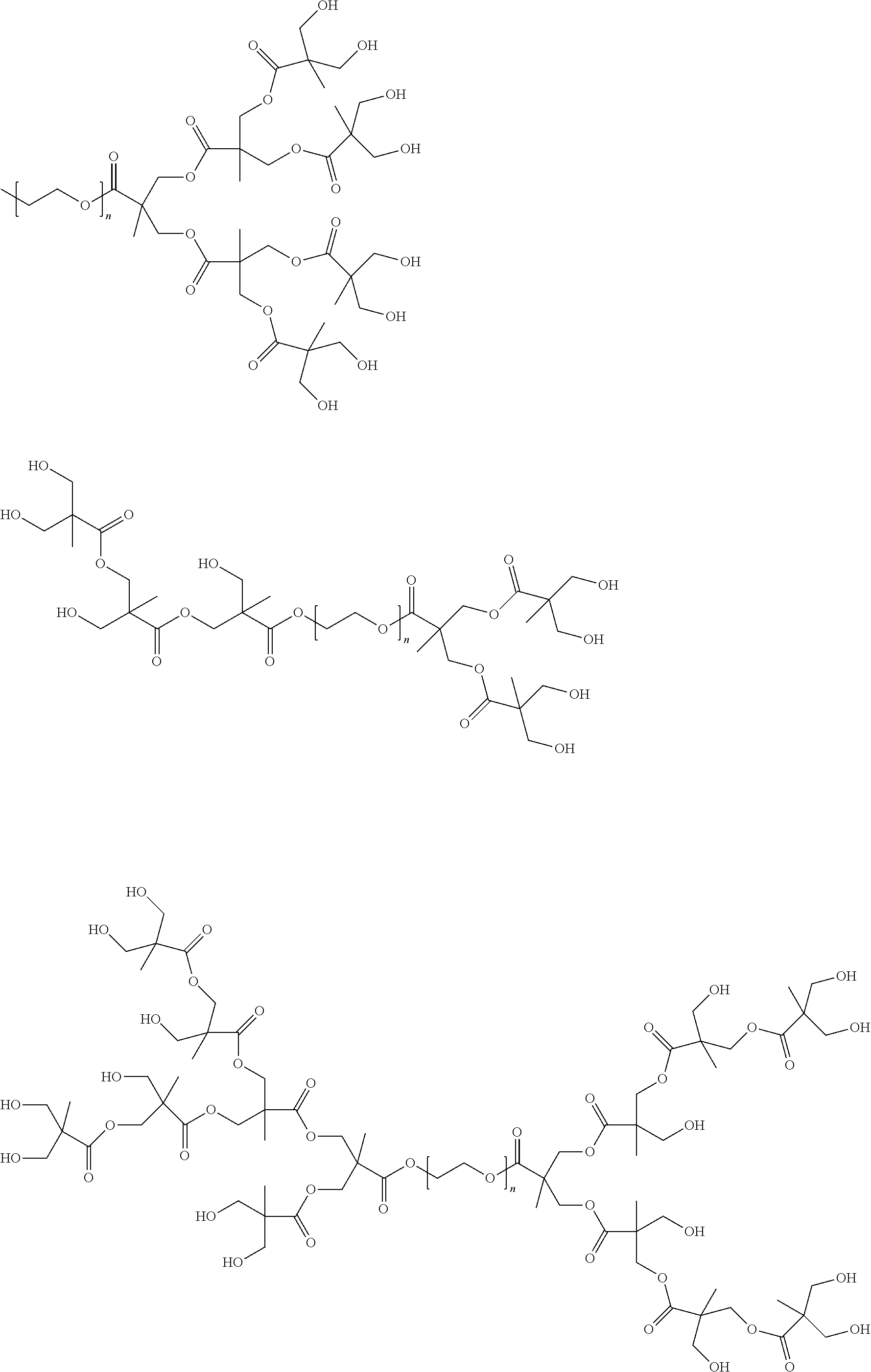

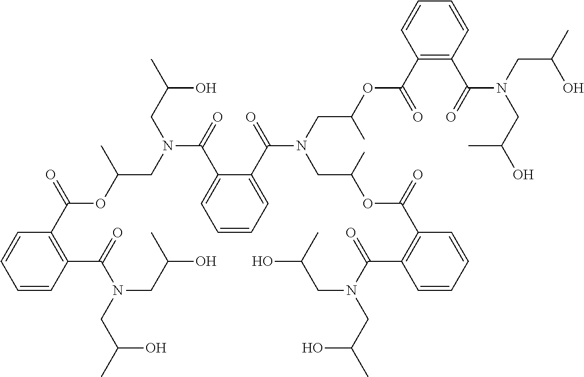 [SMILES: CC(O)CN(CC(C)O)C(=O)c1ccccc1C(=O)OC(C)CN(CC(C)O)C(=O)c1ccccc1C(=O)N(CC(C)OC(=O)c1ccccc1C(=O)N(CC(C)O)CC(C)O)CC(C)OC(=O)c1ccccc1C(=O)N(CC(C)O)CC(C)O]

Preferably, the at least one alditol is selected from the group consisting of threitol, erythritol, galactitol, mannitol, ribitol, sorbitol, xylitol, arabitol, isomalt, lactitol, maltitol, altritol, iditol, maltotritol, and hydrogenated oligo- and polysaccharides with polyol end s groups, and mixture thereof. It is especially preferred for the at least one preferred alditol to be selected from the group consisting of erythritol, mannitol, isomalt, maltitol and mixture thereof.

Other example of suitable sugar alcohols are heptitols and octitols: meso-glycero-allo-heptitol, D-glycero-D-altro-heptitol, D-glycero-D-manno-heptitol, meso-glycero-gluco-heptitol, D-glycero-D-galacto-heptitol (perseitol), D-glycero-D-gluco-heptitol, L-glycero-D-gluco heptitol, D-erythro-L-galacto-octitol, D-threo-L-galacto-octitol.

In particular, the at least one cyclitol can be selected from the group consisting of inositol (myo-, scyllo-, D-chiro-, L-chiro-, muco-, neo-, allo-, epi-, and cis-inositol), 1,2,3,4-tetrahydroxycyclohexane, 1,2,3,4,5-pentahydroxycyclohexane, quercitol, viscumitol, is bornesitol, conduritol, ononitol, pinitol, pinpollitol, quebrachitol, ciceritol, quinic acid, shikimic acid and valienol, with myo-inositol being preferred.

Other suitable costabilisators are ester and ether derivatives of the mentioned alditols or cyclitols, such as, e.g., the following compounds:

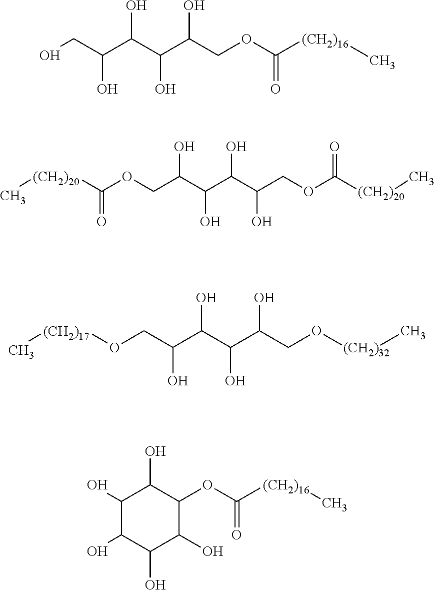

Examples of suitable UV absorbers are compounds based on 2-2′-hydroxyphenyl)benzotriazolene, 2-hydroxybenzophenones, esters of, benzoic acid benzoic s acids, acrylates, oxamides, and 2-(2-hydroxyphenyl)-1,3,5-triazinenes.

Examples of suitable 2-(2′-hydroxyphenyl)benzotriazoles are 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxy-phenyl)benzotriazole, 2-(2′-hydroxy-5′-(1,1,3,3-tetramethyl butyl)phenyl)benzotriazole, 2-(3′,5′-di-tert-butyl-2′-hydroxyphenyl)-5-chlorobenzotriazole, 2-(3′″-tert-butyl-2′-hydroxy-5′-methylphenyl-5-chlorobenzotriazole, 2-(3′-sec-butyl-5′-tert-butyl-2′-hydroxy-phenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(a,a-dimethylbenzyl)-2′-hydroxyphenyl)benzotriazole, 2-(3′-tert-butyl-2′-hydroxy-5′-(2-octyloxycarbonylethyl)phenyl)-5-chlorobenzotriazole, 2-(3′-tert-butyl-5′-[2-(2-1s ethyl hexyloxy)-carbonylethyl]-2′-hydroxyphenyl)-5-chlorobe nzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazol-2-ylphenol]: the transesterification product of 2-[3′-tert-butyl-5′-(2-methoxycarbonylethyl)-2′-hydroxyphenyl]-2H-benzotriazole with polyethyleneglycol 300; [R-CH₂CH₂—COO—CH₂CH₂]2, wherein R=3′-tert-butyl-4′-hydroxy-5′-2H-benzotriazol-2-ylphenyl, 2-[2′-hydroxy-3′-(a,a-dimethylbenzyl)-5′-(1,1,3,3-tetramethylbutyl)-phenyl]benzotriazole, 2-[2′-hydroxy-3′-(1,1,3,3-tetramethylbutyl)-5′-(α,α-dimethylbenzyl)phenyl]benzotriazole.

Examples of suitable 2-hydroxybenzophenones are 4-hydroxy-, 4-methoxy-, 4-octyloxy-, 4-decyloxy-4-dodecyloxy, 4-benzyloxy, 4,2′,4′-trihydroxy-, and 2′-hydroxy-4,4′-dimethyoxy- derivatives of 2-hydroxybenzophenones.

Examples of suitable acrylates are ethyl-a-cyano-β,β-diphenylacrylat, Isooctyl-a-cyano-β,β-diphenylacrylate, methyl-a-carbomethoxycinnamate, methyl-a-cyano-β-methyl-p-methoxycinnamate, butyl-a-cyano-β-methyl-p-methoxycinnamate, methyl-a-carbomethoxy-p-methoxycinnamate, and N-(β-carbomethoxy-β-cyanovinyl)-2-methylindoline.

Examples of suitable esters of benzoic acids are 4-tert-butylphenylsalicylate, phenylsalicylate, octylphenylsalicylate, dibenzoylresorcinol, bis(4-tert-butylbenzoyl)resorcinol, benzoylresorcinol, 2,4-di-tert-butylphenyl-3,5-di-tert-butyl-4-hydroxybenzoate, hexadecyl-3,5-di-tert-butyl-4-hydroxybenzoate, octadecyl-3,5-di-tert-butyl-4-hydroxybenzoate, 2-methyl-4,6-di-tert-butylphenyl-3,5-di-tert-butyl-4-hydroxybenzoate.

Examples of suitable oxamides are 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide, and mixtures of it with 2-ethoxy-2′-ethyl-5,4′-di-tert-butoxanilide, mixtures of o- and p-methoxy-disubstituted oxanilides and mixtures of o- and p-ethoxy-disubstituted oxanilides.

Examples of suitable 2-(2-hydroxyphenyl)-1,3,5-triazines are 2,4,6-tris(2-hydroxy-4-octyloxyphenyl)-1,3,5-triazine, 2-2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)-phenyl]-4,6-bis(2,4-dimethyl) 1,3,5-triazine, 2-[2-hydroxy-4-(2-hydroxy-3-octyloxypropyloxy)phenyl]-4,6-bis(2,4-dimethyl)-1,3,5-triazine, 2-[4-dodecyloxy/tridecyloxy-2-hydroxypropoxy)-2-hydroxyphenyl]-4,6-bis(2,4-dimethylphenyl)-1,3,5-triazine, 2-[2-hydroxy-4-2-hydroxy-3-dodecyloxypropoxy)phenyl]-4,6-bis(2,4-dimethylphenyl-1,3,5-triazine, 2-2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1, 3,5-triazine, 2-{2-hydroxy-4-[3-(2-ethylhexyl-1-oxy)-2-hydroxypropyloxy]phenyl}-4,6-bis(2,4-dimethylphenyl-1,3,5-triazine.

Examples of suitable metal deactivators are N,N′-diphenyloxamide, N-salicylal-N′-salicyloylhydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis-(benzylidene)oxalyldihydrazide, oxanilide, isophthaloyldihydrazide, sebacoylbisphenylhydrazide, N,N′-diacetyladipoyldihydrazide, N,N′-bis(salicyloyl)oxylyldihydrazide, N,N′-bis(salicyloyl)thiopropionyldihydrazide, tris[2-tert-butyl-4-thio(2′-methyl-4′-hydroxy-5′-tert-1-butyl)-phenyl-5-methyl] phenylphosphites.

Especially preferred metal deactivators are:

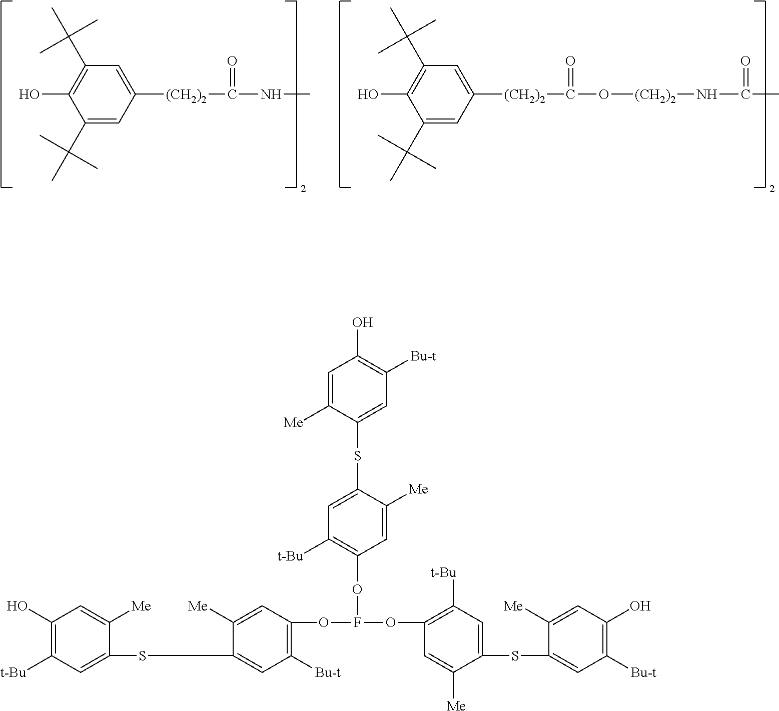

Examples of sterically hindered amine are 1,1-bis(2,2,6,6-tetramethyl-4-piperidyl)succinate, bis(1,2,2,6,6-pentamethyl-4-piperidyl)sebazate, bis(1-octyloxy-2,2,6,6-tetramethyl-4-piperidyl)sebacate, bis(1,2,2,6,6-pentamethyl-4-piperidyl)-n-butyl-3,5-di-tert-butyl-4-hydroxybenzylmalonate, the condensation product of 1-(2-hydroxyethyl)-2,2,6,6-tetramethyl-4-hydroxy-piperidine and succinic acid, linear or cyclic condensation products of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)-nitrilotriacetate, tetrakis(2,2,6,6-tetramethyl-4-piperidyl)-1,2,3,4-butantetracarboxylate, 1,1′-(1,2-ethandiyl)-bis(3,3,5,5-tetramethylpiperazinone), 4-benzoyl-2,2,6,6-tetramethylpiperidine, 4-stearyloxy-2,2,6,6-tetramethylpiperidine, linear or cyclic condensation products of N,N′-bis(2,2,6,6-tetramethyl-4-piperidyl)hexamethylenediamine and 4-morpholino-2,6-dichloro-1,3,5-triazine, the reaction product of 7,7,9,9-tetramethyl-2-cycloundecyl-1-oxa-3,8-diaza-4-oxospiro-[4,5]decane and epichlorhydrin.

Especially preferred hindered amines are the following:

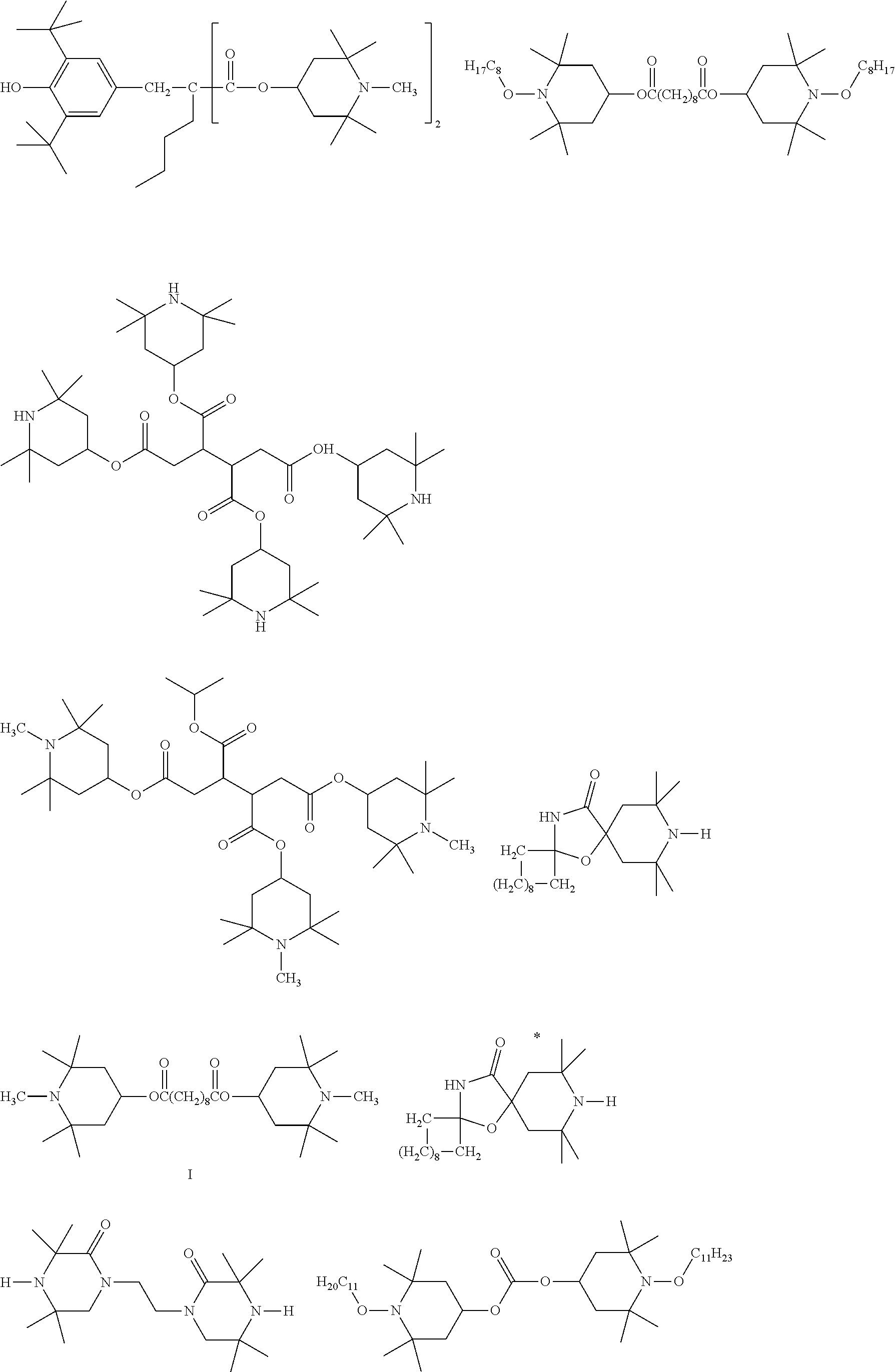

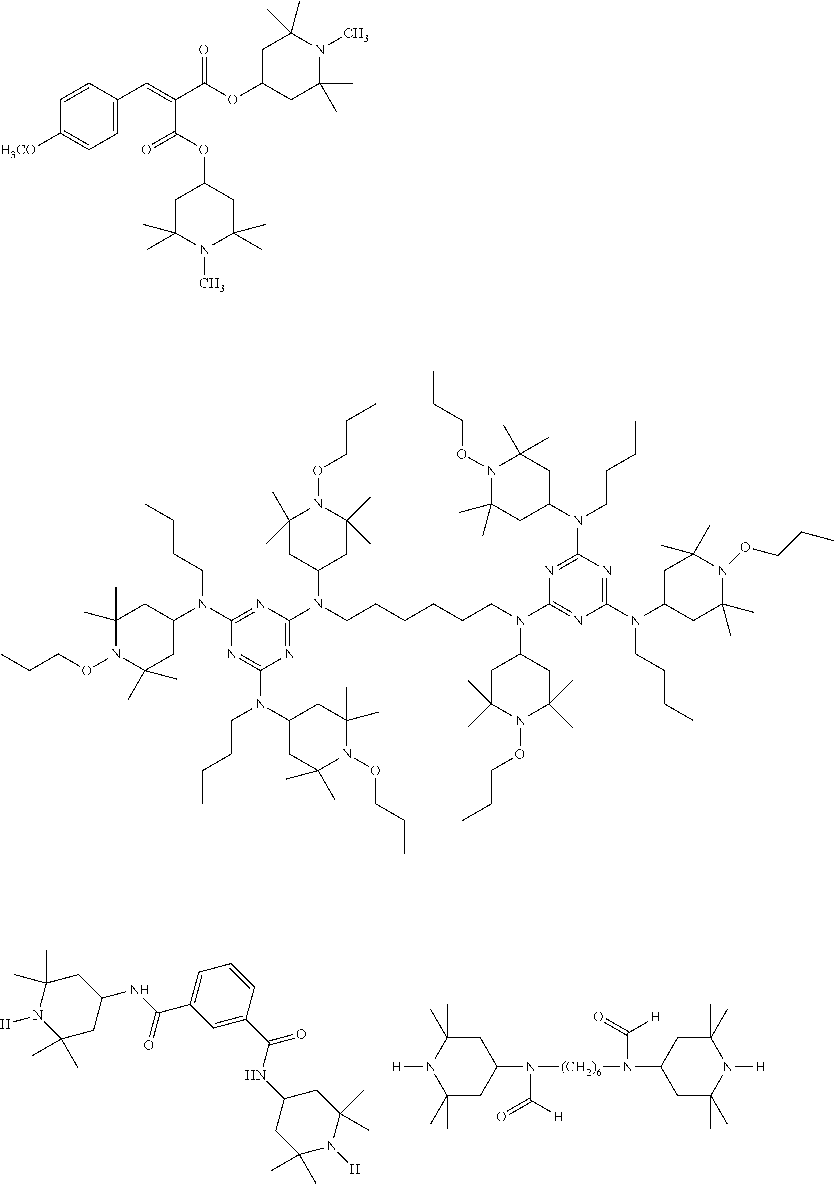

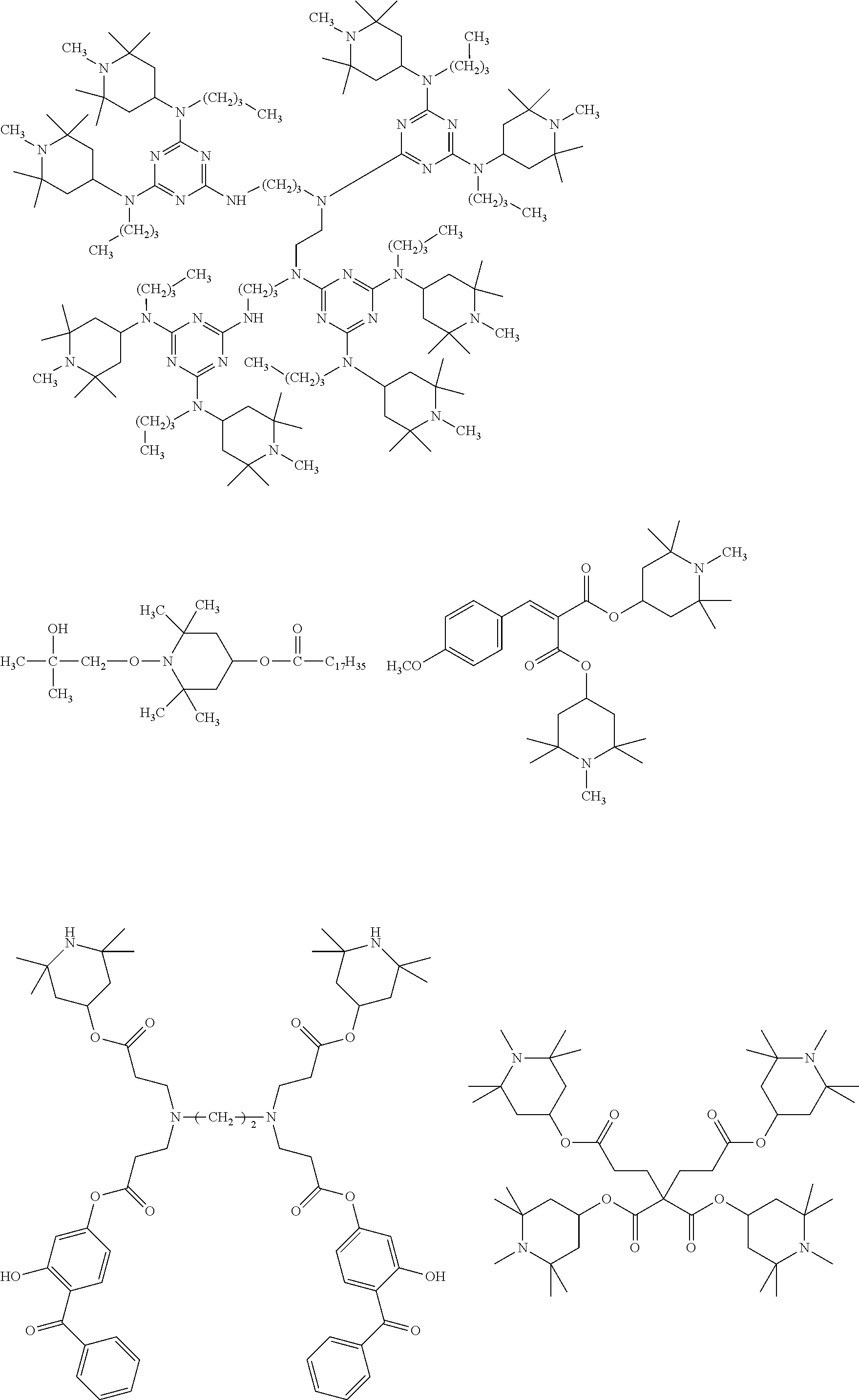

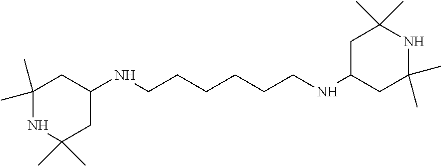

Preferred oligomers and polymers of hindered amines have the following structures:

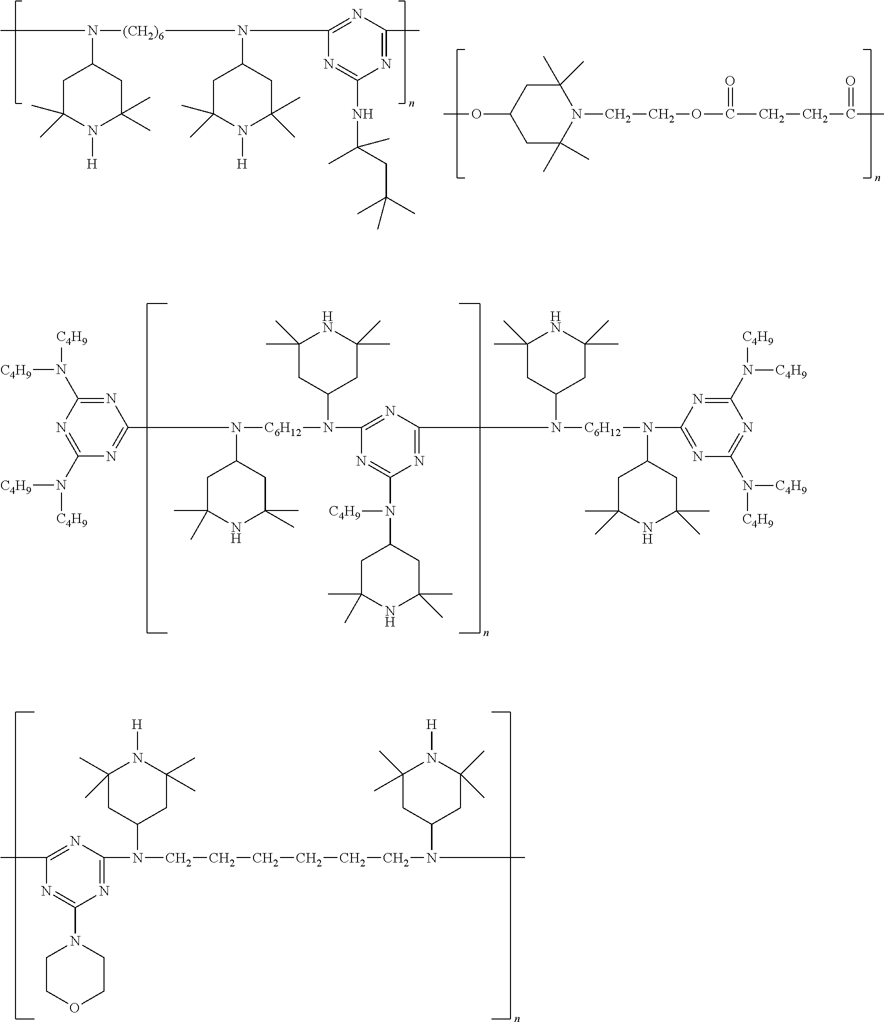

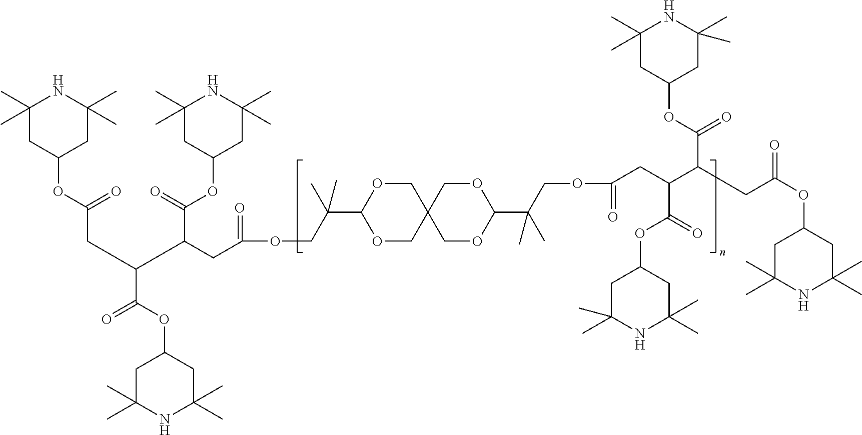

where in each case n=2 to 100, preferably 2 to 10

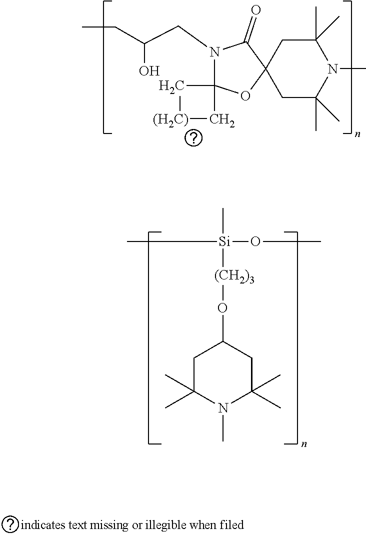

where in each case n=2 to 100, preferably 3 to 20

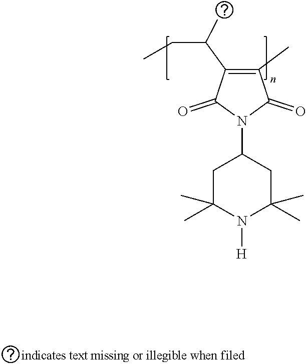

where in each case n=3 to 200, preferably 5 to 100

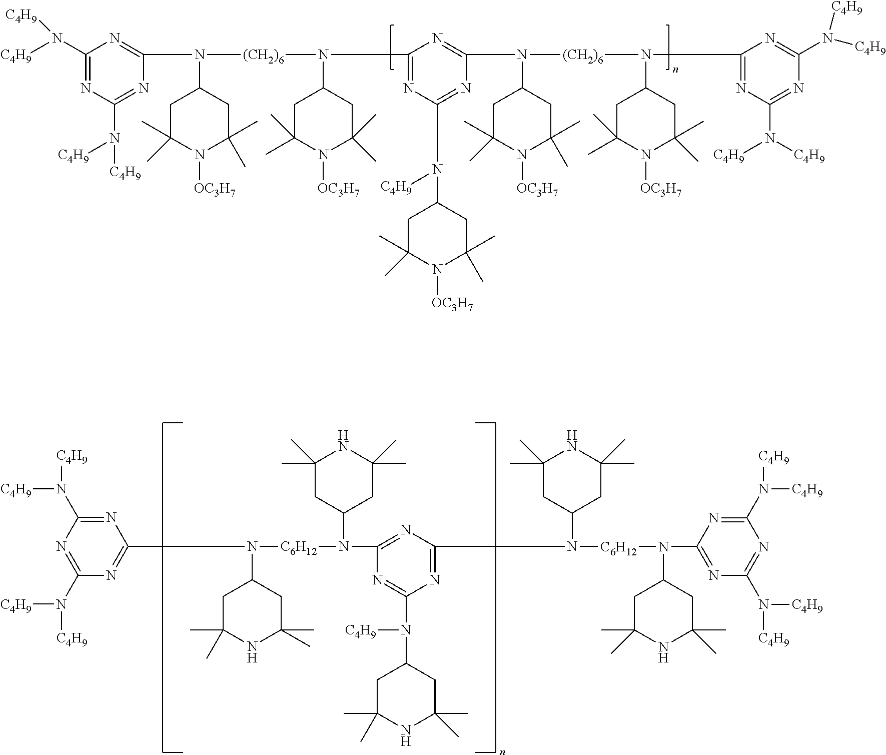

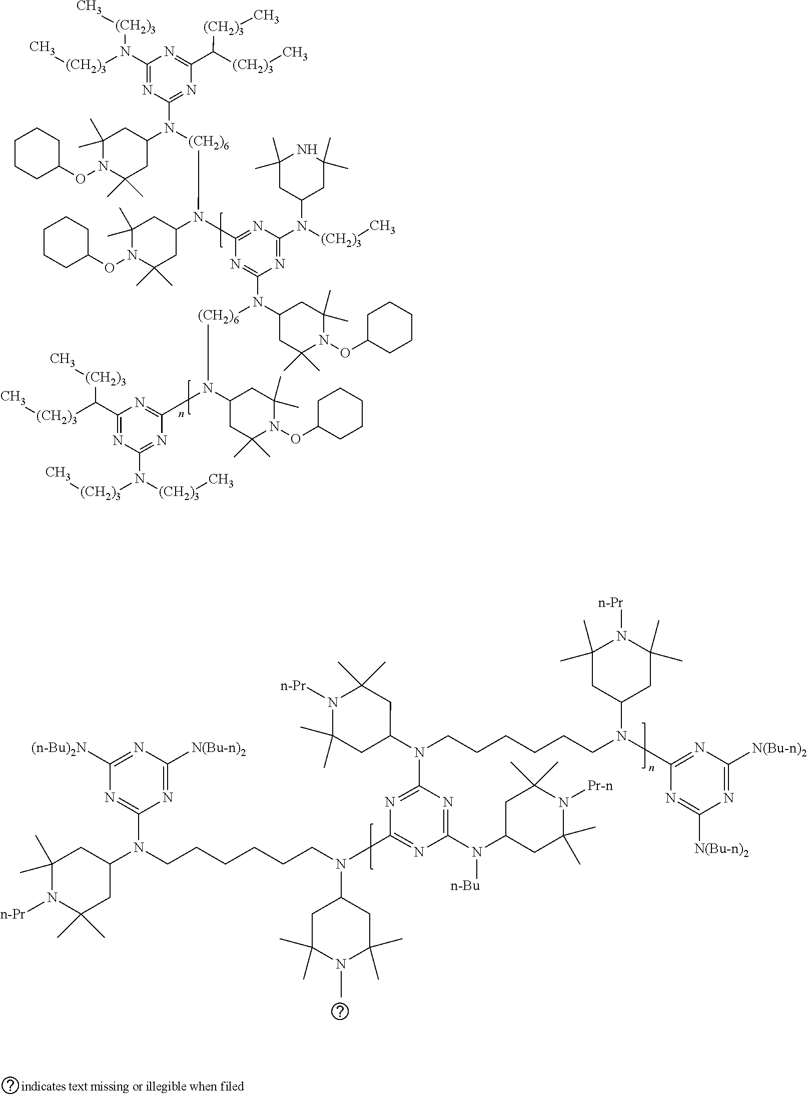

where in each case n=1 to 100, preferably 2 to 10,
or

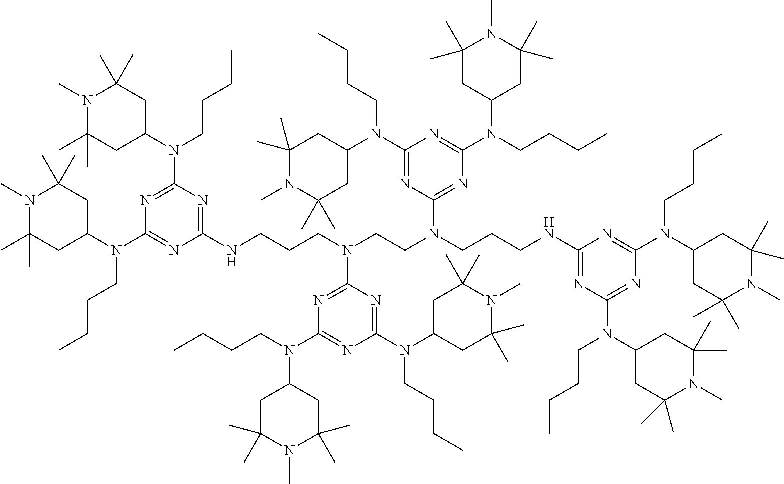

Another suitable light stabilizer is Hostanox NOW (manufacturer: Clariant SE) having the following general structure:

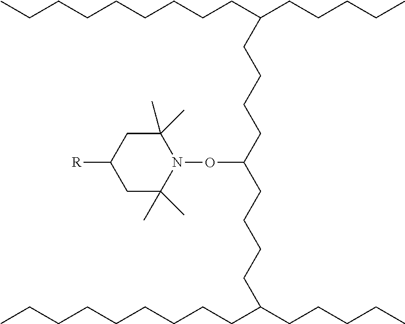

wherein R means —O—C(O)—C₁₅H₃₁ or —O—C(O)—C₁₇H₃₅.

Compatibilizers are used, for example in thermodynamically immiscible blends or also in recyclate mixtures, and contain structural elements of the respective blend components that are mixed. Examples of suitable compatibilizers for polyolefin mixtures are olefin block copolymers consisting of ethylene, propylene, and alpha-olefins, such as, e.g., 1-octene. Other compatibilizers especially to compatibilize polar polymers, such as PET or polyamides, with nonpolar polymers, such as PP or PE, frequently contain reactive groups derived, e.g., from maleic anhydride, acrylic acid, glycidyl acrylate or glycidyl methacrylate and are, for example, polypropylene-g-maleic anhydride, polyethylene-g-maleic anhydride, polypropylene-g-acrylic acid, polyethylene-g-acrylic acid, polyethylene-co-maleic anhydride), SBS-g-maleic anhydride, SEBS-g-maleic anhydride, polyethylene-polyacrylate-polyglycidylmethacrylate.

Examples of suitable dispersants are:
Polyacrylates, e.g., copolymers with long-chain side groups, polyacrylate block copolymers, alkylamides: e.g., N,N′-1,2-ethanediylbisoctadecanamide sorbitan esters, e.g., monostearylsorbitan esters, titanates and zirconates, reactive copolymers with functional groups, e.g., polypropylene-co-acrylic acid, polypropylene-co-maleic anhydride, polyethylene-co-glycidylmethacrylate, polystyrene-alt-maleic anhydride-polysiloxanes: e.g., dimethylsilandiol-ethylene oxide copolymer, polyphenylsiloxane copolymer, amphiphilic copolymers: e.g., polyethylene block polyethyleneoxide, dendrimers, e.g., dendrimers containing hydroxyl groups.

Especially suitable flame retardants are
a) inorganic flame retardants such as, e.g., Al(OH)₃, Mg(OH)₂, AlO(OH), MgCO₃ layer silicates such as, e.g., montmorillonite or sepiolite, unmodified or organically modified, double salts, such as, e.g., Mg—Al-silicates, POSS (polyhedral oligomeric silsesquioxane) compounds, huntite, hydromagnesite or halloysite, as well as Sb₂O₃, Sb₂O₅, MoO₃, zinc stannate, zinc hydroxystannate,
b) nitrogen-containing flame retardants, such as, e.g., z.B. melamine, melem, melam, melon, melamine derivatives, melamine condensation products or melamine salts, benzoguanamine, polyisocyanurates, allantoin, phosphacenes, especially melamine cyanurate, melamine phosphate, dimelamine phosphate, melamine pyrophosphate, melamine polyphosphate, melamine-metal-phosphates, such as, e.g., melamine aluminum phosphate, melamine zinc phosphate, melamine magnesium phosphate, and the corresponding pyrophosphates and polyphosphates, poly-[2,4-(piperazin-1,4-yl)-6-morpholin-4-yl)-1,3,5-triazine], ammonium polyphosphate, melamine borate, melamine hydrobromide,
c) radical formers such as, e.g., alkoxyamines, hydroxylamine esters, azo compounds, sulfenamides, sulfenimides, dicumyl or polycumyl, hydroxyimides and their derivatives such as, e.g., hydroxyimide esters or hydroxyimide ethers
d) Phosphorus-containing flame retardants such as, e.g., red phosphorus such as, e.g., resorcin diphosphate, bisphenol-A-diphosphate and their oligomers, triphenylphosphate, ethylenediamine diphosphate, phosphinates such as, e.g., salts of hypophosphorous acid and their derivatives such as alkylphosphinate salts, e.g., diethylphosphinate aluminum or diethylphosphinate zinc or aluminum phosphinate, aluminum phosphite, aluminum phosphonate, phosphonate ester, oligomeric and polymeric derivatives of methanephosphonic acid, 9,10-dihydro-9-oxa-10-phosphorphenanthrene-10-oxide (DOPO) and their substituted compounds,
e) halogen-containing flame retardants based on chlorine and bromin such as, e.g., polybrominated diphenyloxides such as, e.g., decabromodiphenyloxide, tris(3-bromo-2,2-bis(bromomethy)propyl-phosphate, tris(tribromoneopentyl)phosphate, tetrabromophthalic acid, 1,2-bis(tribromophenoxy)ethane, hexabromocyclododecane, brominated diphenylethane, tris-(2,3-dibromopropyl)isocyanurate, ethylene-bis-(tetrabromophthalimide), tetrabromo-bisphenol A, brominated polystyrene, brominated polybutadien or polystyrene-brominated polybutadiene copolymers, brominated polyphenylene ether, brominated epoxy resin, polypentabromobenzylacrylate, possibly in combination with Sb₂O₃ and/or Sb₂O₅,
f) Borates such as, e.g., zinc borate or calcium borate, possibly on a substrate such as, e.g., silica
g) Sulfur-containing compounds such as, e.g., elemental sulfur, disulfides and polysulfides, thiuram sulfide, dithiocarbamates, mercaptobenzothiazole, and sulfenamides,
h) Antidrip agents such as, e.g., polytetrafluoroethylene,
i) Silicon-containing compounds such as, e.g., polyphenylsiloxanes,
j) Carbon modifications, such as, e.g., carbon nanotubes (CNT), expandable graphite or graphene
k) as well as combinations or mixtures thereof.

Especially suitable flame retardant are:
radical formers, preferably selected from the group consisting of N-alkoxyamines, —C—C— radical formers, radical formers with azogroups (—N═N—), radical formers with hydrazine groups (—NH—HN—), radical formers with hydrazone groups (>C═N—NH—), radical formers with azine groups (>C═N—N═C<), radical formers with triazene groups (—N═N—N<), or from the group of iminoxytriazines.

The preparation of suitable azo compounds is described, for example, in M. Aubert et. al. Macromol. Sci. Eng. 2007, 292, 707-714 or in WO 2008101845; the preparation of hydrazones and azines in M. Aubert et al., Pol. Adv. Technol. 2011, 22, 1529-1538; the preparation of triazenes in W. Pawelec et al., Pol. Degr. Stab. 2012, 97, 948-954. The synthesis of iminoxytriazines is described in WO 2014/064064.

Radical formers that are especially to be used are selected from the group consisting of
a) N-alkoxyamines having the following structural formula

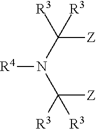

wherein
R³ stands for hydrogen or a possibly substituted alkyl-, cycloalkyl-, aryl-heteroaryl- or acyl- group, especially a C1 to C4- alkyl group,
R⁴ stands for an alkoxy-, aryloxy-, cycloalkoxy-, aralkoxy-, or acyloxy- group,
Z stands for hydrogen or a possibly substituted alkyl-, cycloalkyl-, aryl-heteroaryl- or acyl- group, the two Z groups also being able to form a closed ring, which can possibly be substituted by ester-, ether-, amine, amide, carboxy- or urethane groups,
E stands for an alkoxy-, aryloxy-, cycloalkoxy-, aralkoxy-, or acyloxy- group,
b) Azo compounds having the structural formulas depicted below

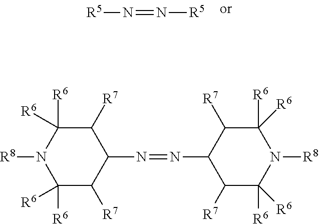

wherein
R⁵ means an alkly-, cycloalkyl-, or aryl group,
R⁶ is the same or different for every occurrence, and means a linear or branched alkyl group,
R⁷ is the same or different for every occurrence, and means a hydrogen or a linear or branched alkyl group, and
R⁸ is the same or different for every occurrence, and means an alkyl, alkoxy-, aryloxy-cycloalkyloxy-, aralkoxy or acyloxy group,
c) dicumyl having the structural formula depicted below

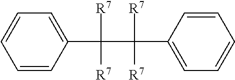

wherein R⁷ has the previously indicated meaning, preferably being methyl,
d) and/or poly cumyl having the structural formula depicted below

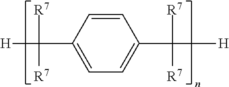

wherein R⁷ has the previously indicated meaning, preferably being methyl, and 2<n<100.

Typical examples of the previously mentioned N-alkoxyamines having the indicated structure are:
1-cyclohexyloxy-2,2,6,6-tetramethyl-4-octadecylaminopiperidine: bis(1-octyloxy-2,2,6,6-tetramethylpiperidin-4-yl) sebacate; 2,4-bis[(1-cyclohexyloxy-2,2,6,6-tetramethylpiperidin-4-yl)butylamino]-6-(2-hydroxyethylamino-S-triazine; bis(1-cyclohexyloxy-2,2,6,6-tetramethylpiperidin-4-yl) adipate;
2,4-bis[(1-cyclohexyloxy-2,2,6,6-tetramethylpiperidin-4-yl)butylamino]-6-chlor-S-triazine; 1-(2-hydroxy-2-methylpropoxy)-4-hydroxy-2,2,6,6-tetramethylpiperidine; 1-(2-hydroxy-2-methylpropoxy)-4-oxo-2,2,6,6-tetramethylpiperidine; 1-(2-hydroxy-2-methylpropoxy)-4-octadecanoyloxy-2,2,6,6-tetramethylpiperidine; bis(1-(2-hydroxy-2-methylpropoxy)-2,2,6,6-tetramethylpiperidin-4-yl)sebacate; bis(1-(2-hydroxy-2-methylpropoxy)-2,2,6,6-tetramethylpiperidin-4-yl)adipate; 2,4-bis{N-[1-(2-hydroxy-2-methylpropoxy)-2,2,6,6-tetramethylpiperidin-4-yl]-N-butylamino}-6-(2-hydroxyethylamino)-S-triazine); 4-piperidinol, 2,2,6,6-tetramethyl-1-(undecyloxy)-4,4′-carbonate; the reaction product of 2,4-bis[(1-cyclohexyloxy-2,2,6,6-tetramethylpiperidin-4-yl)butylamino]-6-chloro-S-triazine with N,N′-bis(3-aminopropylethylenediamine); the oligomeric compound that is the condensation product of 4,4′-hexamethylene-bis(amino-2,2,6,6-tetramethylpiperidine) and 2,4-dichloro-6-[(1-cyclohexyloxy-2,2,6,6-tetramethyl-4-yl)butylamino]-S-triazine, closed at its ends with 2-chloro-4,6-bis(dibutylamino)-S-triazine; aliphatic hydroxylamine such as, e.g., disterarylhydroxylamine; as well as compounds having the following formulas.

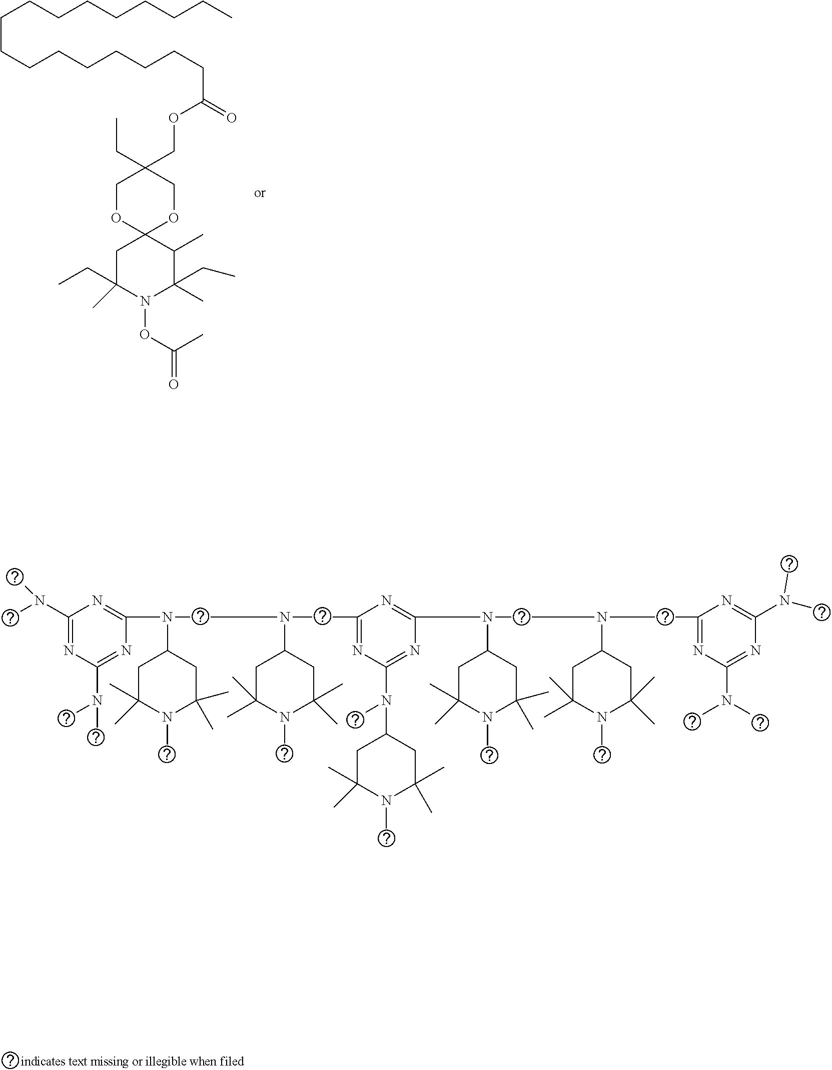

wherein n=1-15

Some of the above-mentioned compounds are commercial products and are sold under the following trade names: FLAMESTAB NOR 116©, TINUVIN NOR 371©, IRGATEC CR 76© from BASF SE, Hostavin NOW® from Clariant or ADK Stab LA 81© from Adeka. Dicumyl and polycumyl are commercial products that are available, e.g., from United Initiators.
b) Phosphorus-containing flame retardants, e.g., phosphinates having the following structures:

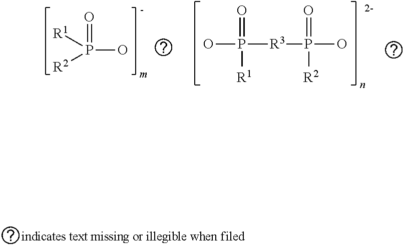

wherein R¹ and R² are preferably identical or different and are selected from the linear or branched C1-C6 alkyl and/or aryl; M is selected from the group consisting of Mg, Ca, Al, Sb, Sn, Ge, Ti, Fe, Zr, Ce, Bi, Sr, Mn, Li, Na, K, Zn and/or a protonated nitrogen base, preferably calcium ions, magnesium ions, aluminum ions, and/or zinc ions; and m=1-4, preferably 2 or 3; n=1-4, preferably 1 or 3; and x=1-4, preferably 1 or 2. In an especially preferred embodiment, R¹=alkyl, R²=alkyl, and M=Al or Zn.

An especially preferred example of a phosphinate is provided by the commercially available products Exolit OP® from Clariant SE.

Other preferred phosphorus-containing flame retardants are metal salts of hypophosphorous acid having a structure according to the formula

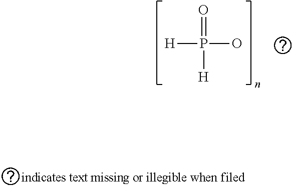

wherein Met is a metal, selected from groups I, II, III, and IV of the periodic table of the elements, and n is a number from 1 to 4, which corresponds to the charge of the corresponding metal ion. Metⁿ⁺, is, for example, Na⁺, Ca²⁺, Mg²⁺, Zn²⁺, Ti⁴⁺ or Al³⁺, with Ca²⁺, Zn²⁺ and Al₃³⁺ being especially preferred.

Some of the above-mentioned salts of hypophosphorous acid are commercially available, e.g., under the name Phoslite® from Italmatch Chemicals.

Another preferred group of phosphorus-containing flame retardants are phosphonates or phosphonic acid diarylesters having a structure according to the following formula:

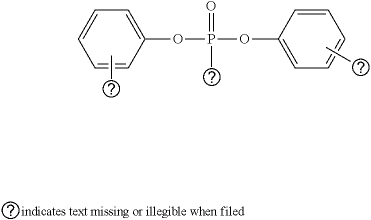

wherein R₈, and R₁₀=H, alkyl, preferably C1-C4, R₉=C1-C4 alkyl u=1-5 and v=1-5.

Corresponding structures can also be in the form of phosphonate oligomers, polymers, and copolymers. Linear or branched phosphonate oligomers and polymers are known from the prior art. For branched phosphonate oligomers and polymers, refer to the U.S. Pat. Nos. 2,716,101, 3,326,852, 4,328,174, 4,331,614, 4,374,971, 4,415,719, 5,216,113, 5,334,692, 3,442,854, 6,291,630 B1, 6,861,499 B2, and 7,816,486 B2. For phosphonate oligomers, refer to the US patent applications US 2005/0020800 A1, US 2007/0219295 A1, and US 2008/0045673 A1. With respect to linear phosphonate oligomers and polymers, refer to the US patent documents U.S. Pat. Nos. 3,946,093, 3,919,363, 6,288,210 B1, 2,682,522, and 2,891,915.

Phophonates are available, for example, under the trade name Nofia® of FRX Polymers.

Another preferred group of phosphorus-containing flame retardants is compounds based on oxaphosphorineoxide and its derivatives having, for example, the following structures:

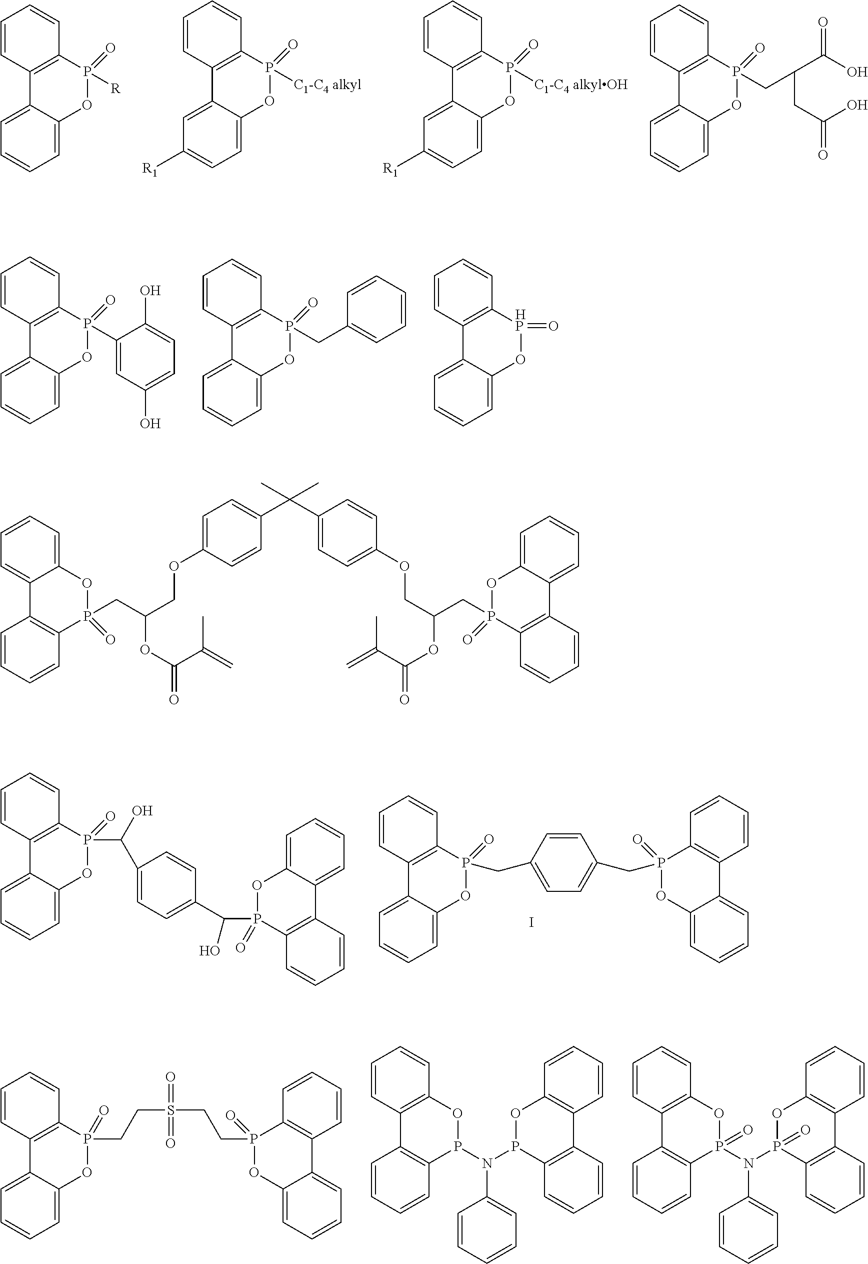

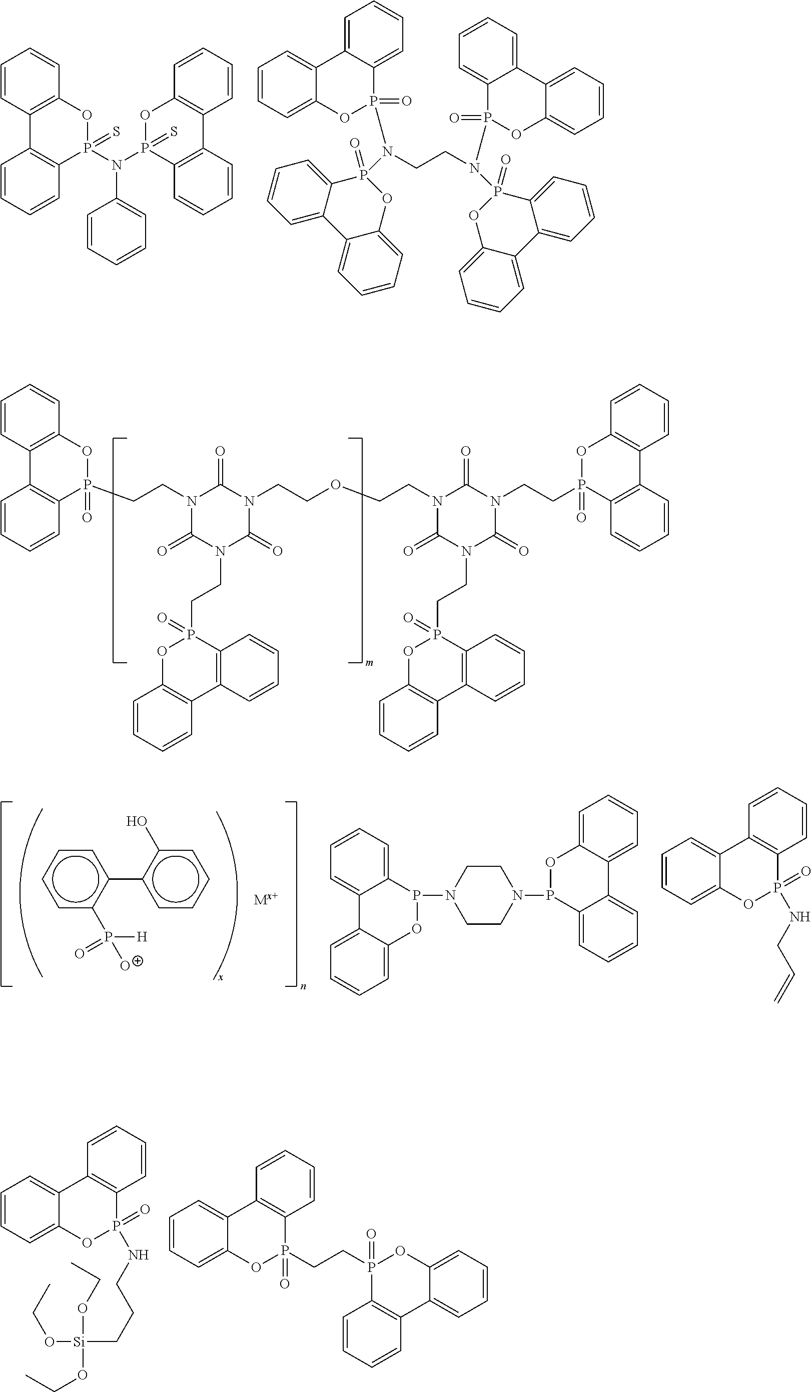

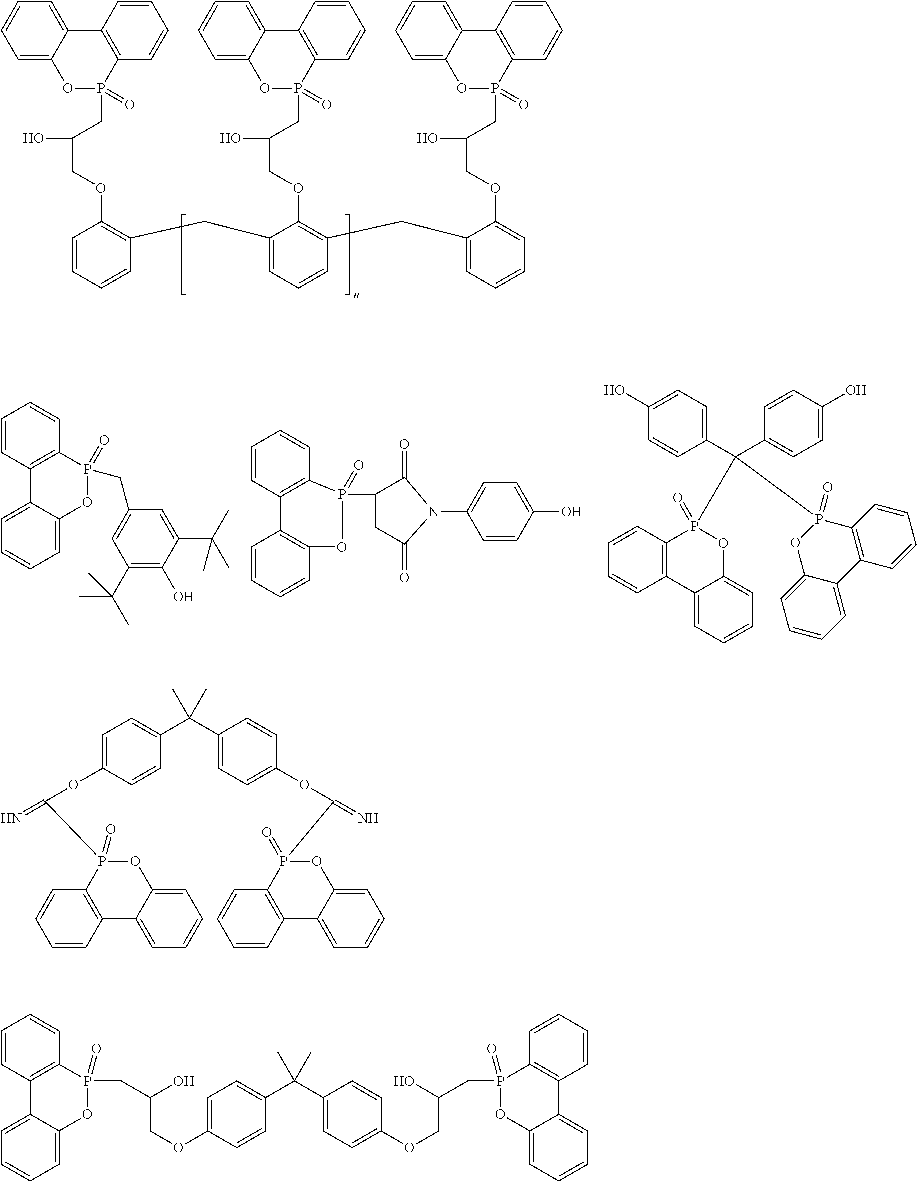

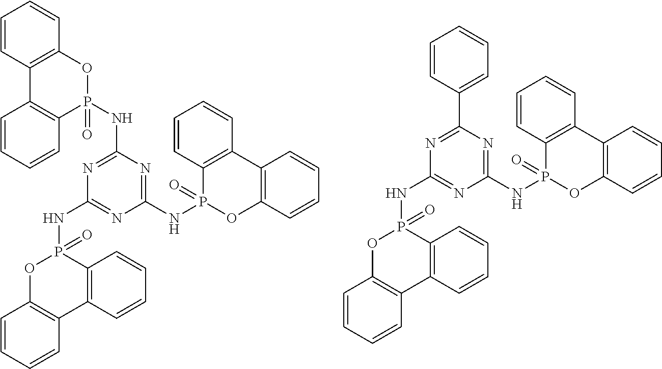

wherein M is a metal selected from the second, third, twelfth, or thirteenth group of the periodic table of the elements, x=2 or 3, n≥10, m=0-25, R=H, a halogen or an aliphatic or aromatic group with 1-32 C atoms, and R₁=H, C1-C6 alkyl or phenyl.

Products based on oxophosphorineoxide are commercially available, for example, under the trade name Ukanol® of Schill and Seilacher GmbH. Other compounds can be prepared, for example, according to the patent specifications WO 2013020696, WO 2010135398, WO03070736, WO2006084488, WO 2006084489, WO 2011000019, WO 2013068437, WO 2013072295.

Other suitable phosphorus-containing flame retardants are cyclic phosphonates having a structure in accordance with one of the following formulas:

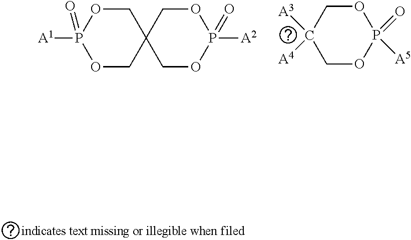

wherein A¹ and A² represent, independently of one another, a substituted or unsubstituted straight-chain or branched alkyl group with 1 to 4 carbon atoms, a substituted or unsubstituted benzyl, a substituted or unsubstituted phenyl, a substituted or unsubstituted naphthyl, and wherein A³ and A⁴ represent, independently of one another, methyl or ethyl and A⁵ is a straight-chain or branched alkyl group with 1 to 4 carbon atoms or a phenyl or benzyl group, each of which can have up to 3 methyl groups.

Cyclic phosphonates are commercially available, for example from the company Thor GmbH under the trade name Aflammit®, or can be prepared in accordance with EP 2450401.

Other synergistic phosphorus-containing flame retardants are phosphacenes, especially polymeric phosphacenes. A corresponding product is commercially available, e.g., from Otsuka Chemicals under the name SPB-100.

a) Nitrogen-containing flame retardants
Preferred nitrogen-containing flame retardants are melaminepolyphosphate, melaminecyanurate, melamine-metal-phosphates, poly-[2,4-(piperazin-1,4-yl)-6-morpholin-4-yl)-1,3,5-triazine] and ammonium polyphosphate. These compounds are commercially available under the trade names Melapur® from BASF SE, Budit® from Chemische Fabrik Budenheim, Exolit® from Clariant, Safire® from Huber Chemicals, or MCA PPM Triazine from MCA Technologies GmbH.

b) Preferred sulfur-containing flame retardants are, for example, the following compounds

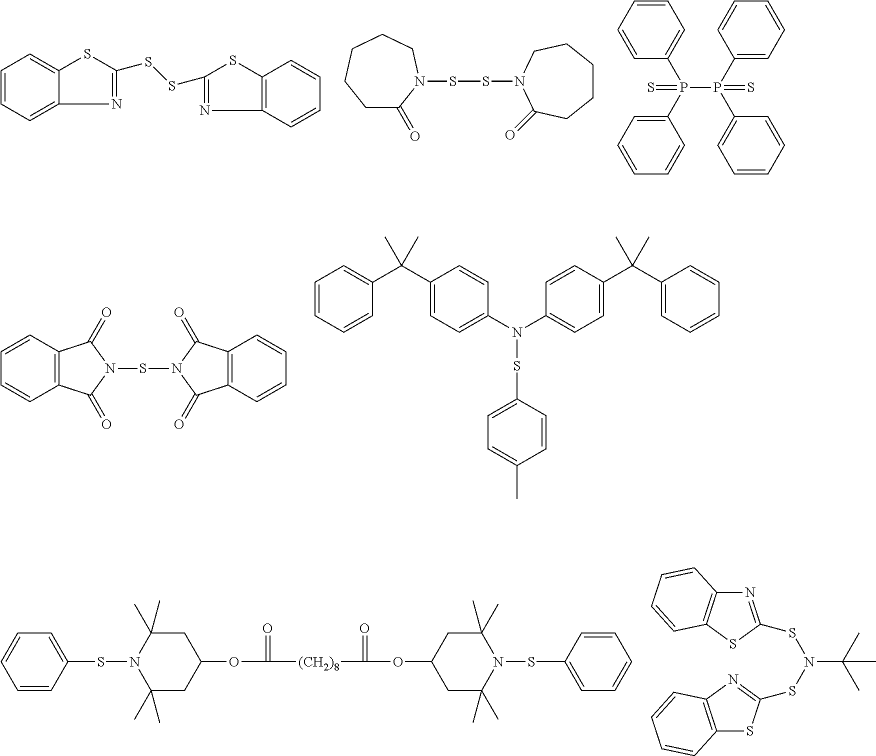

Very especially preferred flame retardants are halogen-free and are the following compounds:
Al(OH)₃, Mg(OH)₂

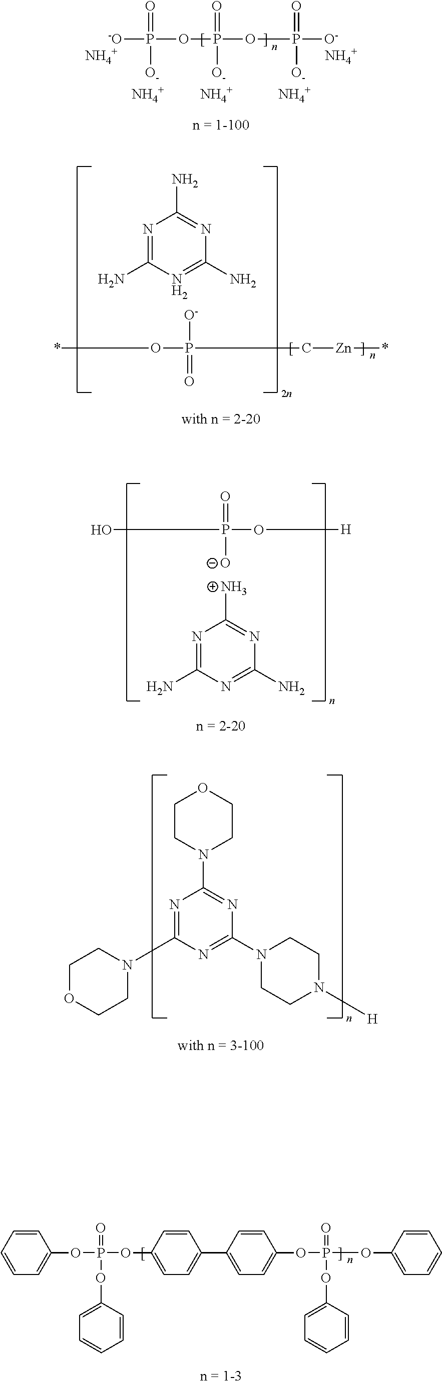

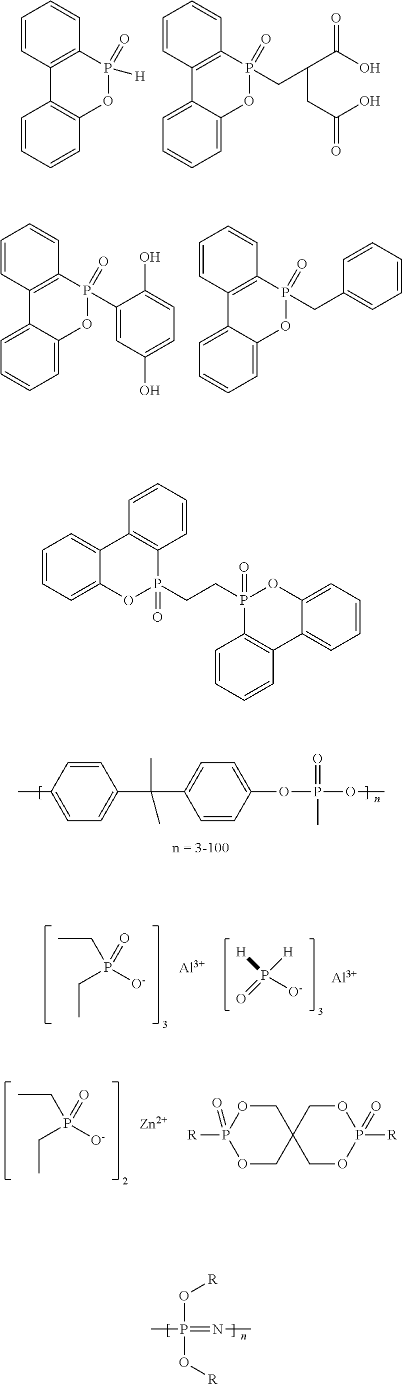

in each case with R=alkyl, phenyl, and n=3-20

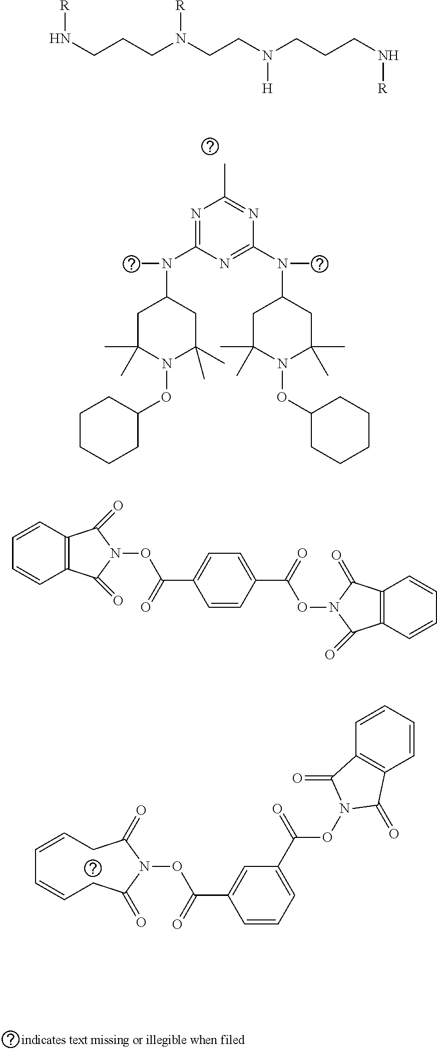

Suitable lubricants and processing aids are, for example, polyethylene waxes, polypropylene waxes, fatty acid salts such as, e.g., calcium stearate, zinc stearate, or salts of montan waxes, amide waxes such as, e.g., erucic acid amide or oleic acid amides, fluoropolymers, silicones or neoalkoxytitanates and zirconates.

Examples of suitable heat stabilizers, especially for PVC recyclates, are metal soaps of divalent metals such as Ba, Zn, Ca, e.g., zinc stearate, calcium stearate, organotin compounds, e.g., methyl and octyl tin compounds such as, e.g., dioctyltinbisisooctylthioglycolate or dioctyltinmaleate, aminouracils, aminocrotonic acid esters, perchlorate salts, as well as costabilisators phosphites, epoxides, polyols, diketones, dihydropyridines, hydrotalcites, zeolites.

Suitable pigments can be of an inorganic or organic nature. Examples of inorganic pigments are titanium dioxide, zinc oxide, zinc sulfide, iron oxide, ultramarine, soot; examples of organic pigments are anthraquinones, anthanthrones, benzimidazolones, quinacridones, diketopyrrolopyrroles, dioxazines, indanthrones, isoindolinones, azo compounds, perylenes, phthalocyanines or pyranthrones. Other suitable pigments are effect pigments based on metals or pearlescent pigments based on metal oxides.

Examples of suitable brighteners are bisbenzoxazoles, phenylcumarins or bis(styryl)biphenyls and especially brighteners having the formulas:

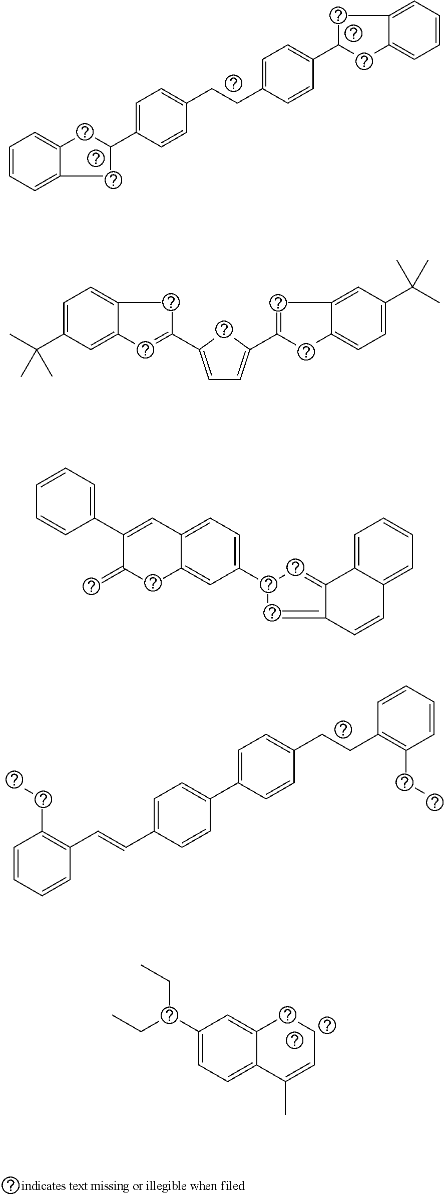

Examples of suitable filler deactivators are polysiloxanes, polyacrylates, especially block copolymers such as polymethacrylic acid-polyalkylene glycol or polyglycidyl(meth)acrylates and their copolymers, e.g., with styrene as well as epoxides, e.g., having the following structures:

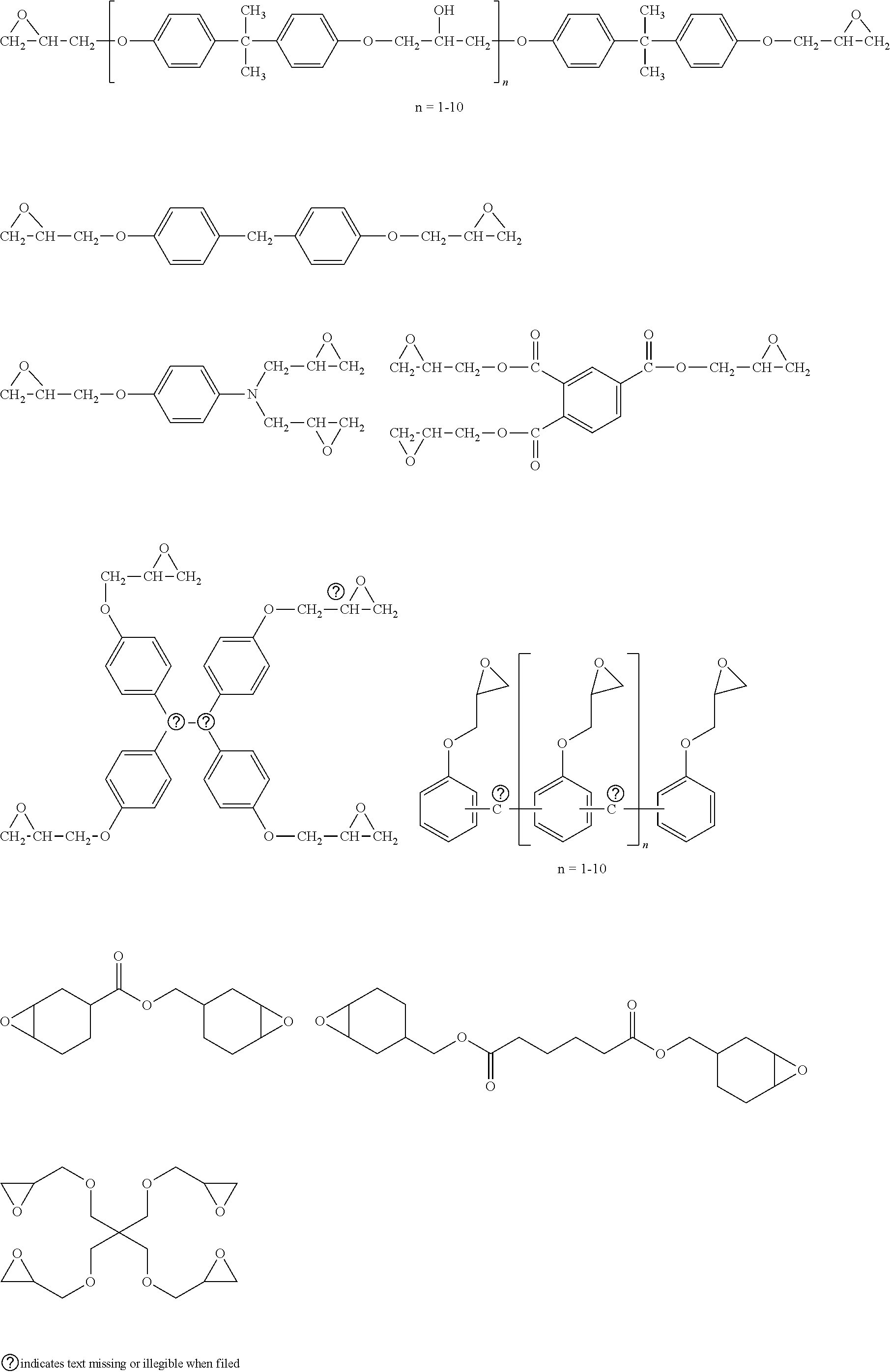

Examples of suitable antistatic agents are ethoxylated alkylamines, fatty acid esters, alkyl sulfonates, and polymers that form a co-continuous network with the polymer matrix such as, e.g., polyether amides, polyester amides, polyetheresteramides or polyether-block copolymers, possibly with the addition of ionically conductive metal salts.

Suitable antiozonants are the above-mentioned amines such as, e.g., N,N′-di-isopropyl-p-phenylenediamine, N,N′-di-sec-butyl-p-phenylenediamine, N,N′-bis(1,4-dimethylpentyl)-p-phenylenediamine, N,N′-dicyclohexyl-p-phenylenediamine, N-Isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine

Examples of suitable rheology modifiers, e.g., for the preparation of controlled rheology polypropylene (CR-PP), are peroxides, alkoxyamine esters, oxyimide sulfonic acid esters and especially the following structures:

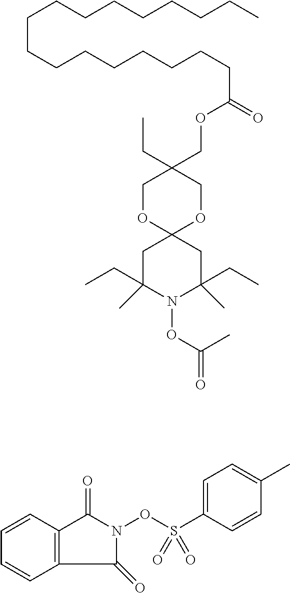 [SMILES: CCCCCCCCCCCCCCCCCC(=O)OCC1(CC)COC2(CC(C)(CC)N(OC(C)=O)C(C)(CC)C2)OC1 ; Cc1ccc(S(=O)(=O)ON2C(=O)c3ccccc3C2=O)cc1]

Suitable additives to increase the molecular weight of polycondensation polymers (chain extenders) are diepoxides, bis-oxazolines, bis-oxazolones, bis-oxazines, diisocyanates, dianhydrides, bis-acyllactames, bis-maleimides, dicyanates, carbodiimides, and polycarbodiimides. Other suitable chain extenders are polymeric compounds such as, e.g., polystyrene-polyacrylate-polyglycidyl(meth)acrylate copolymers, polystyrene-maleic anhydride copolymers, and polyethylene-maleic anhydride copolymers.

Examples of suitable additives to increase electrical conductivity are the mentioned antistatic agents, soot, and carbon compounds such as carbon nanotubes and graphene, metal powders such as, e.g., copper powders and conductive polymers such as, e.g., polypyrroles, polyanilines, and polythiophenes.

Examples of suitable infrared-active additives are aluminum silicates, hydrotalcites, or dyes such as phthalocyanine or anthraquinone.

Examples of suitable cross-linking agents are peroxides such as dialkyl peroxides, alkyl-aryl peroxides, peroxyesters, peroxycarbonates, diacylperoxides, peroxyketals, silanes such as, e.g., vinyltrimethoxysilane, vinyltriethoxysilane, vinyltriacetoxysilane, vinyltris(2-methoxyethoxy)silane, 3-methacryloyloxypropyltrimethoxysilane, vinyldimethoxymethylsilane, or ethylene-vinylsilane copolymers.

Suitable prodegradants are additives that deliberately accelerate or control the breakdown of a polymer in the environment. Examples are transition metal fatty acid esters, e.g., of manganese or iron, which accelerate an oxidative and/or photooxidative breakdown e.g., of polyolefins, or enzymes that induce hydrolytic breakdown, e.g., of aliphatic polyesters.

Examples of suitable chemical propellants are azo compounds such as azodicarboxylic acid amide, sulfonylsemicarbazides such as p-toluolsulfonylsemicarbazide, tetrazoles such as 5-phenyltetrazole, hydrazides such as p-toluolsulfonylhydrazide, 4,4′-oxibis(benzolsulfonyl)hydrazide, N-nitroso compounds such as N,N′-dinitrosopentamethylenetetramine or carbonates such as, e.g., sodium bicarbonate or zinc carbonate.

Examples of suitable slip agents are amide waxes such as wie erucic acid amide or oleic acid amide.

Examples of suitable antiblocking agents are silica, talc, or zeolites.

Examples of suitable antifogging additives are ethoxylated sorbitan esters, ethoxylated fatty acid alcohols, or ethoxylated alkylamine esters.

Examples of suitable biocides are quaternary ammonium salts or silver salts, colloidal silver or silver complexes, or also derivatives of natural substances such as, e.g., chitosan

Suitable aldehyde scavengers are amines, hydroxylamines, poly(vinyl alcohol), zeolites or cyclodextrins, suitable formaldehyde scavengers are melamine derivatives such as, e.g., benzoguanamine or urea derivatives such as allantoin.

Suitable odor binding or odor preventing substances are silicates such as calcium silicate, zeolites or salts of hydroxyfatty acids such as, e.g., z. B. zinc riceneolate.

Examples of suitable marking agents are fluorescent dyes or rare earths.

Suitable nucleation agents are talc, alkali or alkaline earth salts of mono- and polyfunctional carboxylic acids such as, e.g., benzoic acid, succinic acid, adipic acid, e.g. sodium benzoate, zinc glycerolate, aluminum hydroxy-bis(4-tert-butyl)benzoate, 2,2′-methylene-bis-(4,6-di-tert-butylphenyl)phosphate, and trisamides and diamides such as, e.g., trimesic acid tricyclohexylamide, trimesic acid (4-methylcyclohexylamide), trimesic acid tris(tert-butylamide), N,N′,N″-1,3,5-benzoltriyltris(2,2-dimethyl-propanamide), or 2,6-naphthaline dicarboxylic acid dicyclohexylamide.

Suitable clarifiers are especially sorbitol derivatives such as, e.g.,

Suitable antinucleation agents are azine dyes such as, e.g., nigrosine or ionic liquids,

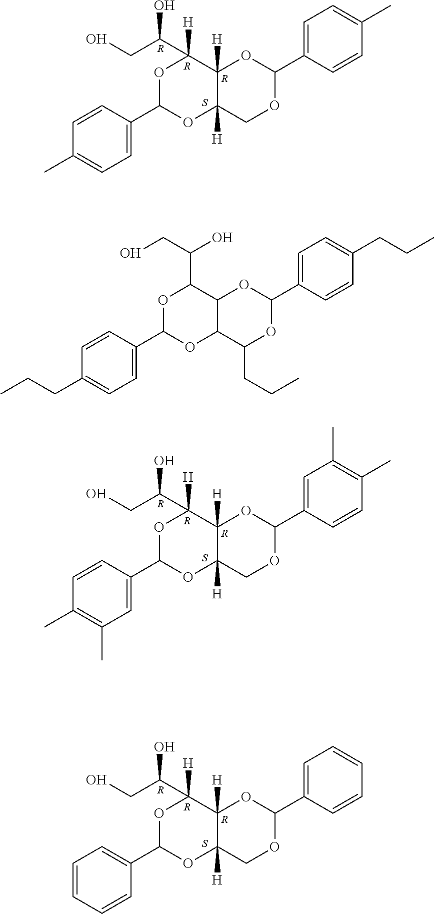

Examples of suitable additives for increasing thermal conductivity of plastic recyclates are is inorganic fillers such as boron nitride, aluminum nitride, aluminium oxide, aluminium silicate. silicon carbide. but also carbon nanotubes (CNT).

Suitable tougheners are usually selected for the respective recyclate and come, for example, from the group of functionalized or nonfunctionalized polyolefins such as, e.g., ethylene copolymers such as EPDM or maleic anhydride or styrene-acrylonitrile-modified EPDM, glycidylmethacrylate-modified ethylene-acrylate-copolymers or also ionomers, core shell polymers based on MBS (methacrylate-butadiene-styrene-copolymer) or acrylate-poly(methylmethacrylate) thermoplastic elastomers (TPE), e.g., based on styrene-block copolymers (styrene-butadiene (SB), styrene-butadiene-styrene (SBS) possibly hydrogenated (SEBS) or modified by maleic anhydride (SEBS-g-MAH), thermoplastic polyurethanes, copolyesters or copolyamides.

Examples of suitable plasticizers are esters of phthalic acid, terephthalic acid, adipic acid, 1,2-cyclohexanedicarboxylic acid, trimellitic acid, citric acid or phosphoric acid such as, e.g., benzyl butyl phthalate (BBP), butyl nonyl phthalate (BNP), didecyl phthalate (DDP), diisobutyl adipate (DIBA), diisodecyladipate (DIDA), dioctyl terephthalate (DOTP), diisotridecyl phthalate (DTDP), tributyl O-acetylcitrate (TBAC), triethyl O-acetylcitrate (TOAC), tetrahydrofurfuryl oleate (THFO), triisooctyl trimellitate (TIOTM), tributyl phosphate (TBP), as well as epoxidized soybean oil (ESO) or epoxidized linseed oil (ELO).

Examples of suitable mold release agents are silicones, soaps, and waxes such as, e.g., montan waxes.

Preferably, the inventive additive, which can be in the form of a powder, liquid; fluid, oil, or which can be compacted on a substrate, or which can be in the form of a granulate, solution or flakes, is mixed with the polymer to be stabilized, the polymer matrix is converted into the melt, and then cooled. Alternatively, it is just as possible to introduce the additive into a polymer melt in a molten state.

Furthermore, the inventive additive compositions can be prepared and introduced in the form of so-called master batches or concentrates, which contain, for example, 10-90% of the inventive compositions in a polymer or in a polymer recyclate.

In another preferred embodiment, the compositions contain secondary antioxidants, especially phosphites/phosphonites, sulfites, acid scavengers, costabilisators based on polyols, and/or light stabilizers from the group of hindered amines (HALS).

In the previously mentioned embodiment, it is advantageous if at least one additive is contained and or added in a quantity from 0.01 to 80 weight %, preferably from 0.01 to 9.99 weight %, more preferably from 0.01 to 4.98 weight %, especially preferably from 0.02 to 2.00 weight %, relative to the sum of the at least one compound having general formula I or II, or in the case of a mixture of multiple compounds having general formula I and/or II, the sum of all compounds having general formula I and/or II of the plastic and of the at least one additive.

Another aspect of this invention relates to a plastic composition containing at least one plastic as well as at least one compound having general formula I and/or II or mixtures of multiple compounds having general formula I and/or II as defined above.

A preferred embodiment provides that the plastic composition have the following composition:
0.01 to 10.00 weight %, preferably from 0.01 to 7.50 weight %, especially preferably from 0.02 to 5.00 weight %, especially preferably from 0.05 to 3.00 weight percent of a compound having general formula I or II, or in the case of a mixture of multiple compounds having general formula I and/or II, the sum of all compounds having general formula I and/or II.
99.99 to 10.00, preferably 99.99 to 90.00 weight %, preferably 99.89 to 95.00 weight %, especially preferably 99.90 to 98.00 weight % of at least one plastic, and
0 to 80.00 weight-%, preferably 0 to 9.99 weight %, more preferably 0.01 to 4.98 weight %, especially preferably 0.02 to 2.00 weight % of at least one additive,
wherein the components add up to 100 weight %.

Here it is preferable that at least one additive be selected from the group consisting of primary and/or secondary antioxidants, especially primary and/or secondary antioxidants selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents and antifogging agents, Preferably, the at least one additive is selected from the group consisting of phosphites, phosphonites, sulfites, polyols, acid scavengers, hindered amines, as well as mixtures and combinations thereof.

Especially preferred plastic compositions consist of
(A) 0.02-2, especially preferably 0.05-1 parts of at least one compound having general formula I or II, or mixtures of multiple compounds having general formula I and/or II as defined above,
(B) 43-99.96 parts of a plastic,
(C) 0.02-4 parts, especially preferably 0.05-2 parts of a
   (a) phosphite or phosphonite and/or
   (b) sulfite and/or
   (c) polyol and/or
   (d) acid scavenger and/or
   (e) hindered amine
(D) 0-50 parts, especially preferably 0-2 parts of another additive,
so as to get 100 parts.

For the case in which the plastic composition (or synonymously polymer composition) has other components added to it, they can be added to the polymers separately, in the form of liquids, powders, granulates, or compacted products, or together with the inventive additive composition (i.e., the at least one compound having general formula I or II or mixtures of multiple compounds having general formula I and/or II, possibly as well as additives), as described above.

The above-described additive composition and possibly the additional additives are incorporated into the plastic by usual processing methods, preferably by mixers, kneaders, or extruders. Preferred processing machines are extruders such as, e.g., single-screw extruders, twin-screw extruders, planetary gear extruders, ring extruders, co-kneaders, which are preferably equipped with vacuum degassing. The processing can be done under air or under inert gas conditions.

The processing of the plastic compositions containing the described additive composition can be done by usual plastic processing methods in continuous and discontinuous processes, such as, e.g., by extrusion, calendering, blow molding, pultrusion, injection molding, pressing, transfer molding, molding, blow molding, rotational molding, deep drawing, sintering, foaming, or also by additive manufacturing processes for preparing granulate, molded parts, semi-finished products, fibers, and films.

Suitable extruders are piston extruders and screw-type extruders, single-screw extruders, twin-screw extruders, multishaft extruders, planetary gear extruders, especially for preparing plastic granulates, tubes, rods, hoses, profiles, jackets, plates, films, V-belts, toothed belts, gaskets, foam sheets (XPS), fibers, and filaments for additive manufacturing processes.

Suitable injection molding machines can have a hydraulic or electromechanical design and can comprise multicomponent injection molding and in-mold processes. Examples of molded parts produced by injection molding are bottles, containers, screw plug boxes, housings, barrels, buckets, pallets, technical parts for autos and transportation such as bumpers, cladding parts, handles, headlight coverings, fittings and functional parts, electrical and electronic applications such as housing parts and accessories of television sets, computers, mobile telephones, washing machines, dish washers, coffee machines, drills, plug-and-socket connectors, storage media, household, leisure, and sports equipment, such as, e.g., flower tubs, coat hangers, game pieces, model making, components for furniture such as, e.g., brackets and clips,

Examples of parts produced by blow molding are especially hollow bodies such as bottles, fuel tanks, canisters, windshield washer fluid reservoirs, and equalizing tanks.

Parts produced by rotational molding are especially tanks such as heating oil and rain water tanks, housing for machines, transportation containers, leisure and watersports items such as, e.g., kayaks.

Calendering is used to produce especially films such as decorative films, wall paper, and floor coverings.

Additive manufacturing processes comprise, for example, binder jetting (BJ), laser sintering (LS), selective laser melting (SLM), electron beam melting (EBM), fused deposition modeling (FDM), fused filament fabrication (FFF), multi-jet modelling (MJM), poly-jet modelling (PJM), layer laminated manufacturing (LLM), thermotransfer sintering (TIS), digital light processing (DLP), photopolymer jetting (PJ), and stereolithography (SL).

Examples of molded parts that can be produced from the inventive composition are foils or films, foams, fibers, cables and pipes, profiles, hollow bodies, tapes, membranes such as, e.g., geomembranes, or adhesives that are produced through extrusion, injection molding, blow molding, calendering, pressing processes, spinning processes, rotomolding, e.g., for packaging, e.g., for foodstuffs, detergents, cosmetics, adhesives in the form of films, bottles, bags, screw plug boxes, storage and transportation containers such as, e.g., boxes, crates, barrels, buckets, pallets, automobile, railroad, airplane, ship, and machine parts, such as, e.g., bumpers, cladding parts, fittings and functional parts, pads, building applications such as profiles, construction films, cable ducts, house claddings, noise protection walls, drainage channels, profile boards, floor coverings, road and landscaping applications, such as, e.g., bases for moveable bollards and signs, stakes, barriers, geotextiles, electric and electronic applications such as housing parts and accessories of television sets, computers, mobile telephones, washing machines, dish washers, coffee machines, drills, plug-and-socket connectors, storage media, cable insulation, pipes for, e.g., water, gas, waste water, irrigation; drainage pipes, hygiene articles such as, e.g., diapers, furniture and textile applications such as, e.g., curtains and cushions, working surfaces, household, leisure, and sports articles such as, e.g., balls, tennis rackets, skis, flower tubs, rain barrels, coat hangers, landscaping applications such as, e.g., mulch, tunnel, or perforated films, plant pots, pharmaceutical and plant protection application such as, e.g., for encapsulation of active ingredients and biologically active substances, in medical engineering for producing suture material, bandaging material, ortheses and prostheses.

Furthermore, the invention relates to a process for stabilizing a plastic composition, especially against oxidative, thermal and/or actinic degradation, in which at least one compound having general formula I or II, or mixtures of multiple compounds having general formula I and/or II as defined above is incorporated into at least one plastic or into a blend of at least two plastics.

In addition, this invention relates to novel compounds in accordance with general formula I or II as defined above.

Another aspect of this invention relates to a stabilizer-composition consisting of
a) at least one compound having general formula I or II or mixtures of multiple compounds having general formula I and/or II as defined above (component A), and
b) at least one additive (component B), selected from the group consisting of primary and/or secondary antioxidants, especially primary and/or antioxidants selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents and antifogging agents,
wherein the at least one additive is preferably selected from the group consisting of phosphites, phosphonites, sulfites, polyols, acid scavengers, hindered amines as well as mixtures and combination thereof.

In the stabilizer-composition, it is preferable if component A and component B are in a weight ratio of 100:1 to 1:100, preferably 10:1 to 1:10, especially preferably from 5:1 to 1:5.

This invention will be explained in detail by the following embodiments, without limiting the invention to the special embodiments presented.

### EXEMPLARY EMBODIMENTS

To test the efficacy of the inventive stabilizers, a commercially available polypropylene (Moplen HP 500N, Lyondell Basell Industries) was homogenized with the inventive stabilizers in a powder-powder mixture, and cycled in a twin-screw microcompounder (MC 5, manufactured by DSM) for 30 minutes at 200° C. and 90 rpm, and the decrease in strength was recorded. The strength is a direct measure pf the molecular weight of polypropylene: the smaller the decrease, the higher the stabilization effect.

The addition of 0.1 to 0.3% of a hexylester of vanilic acid prepared in accordance with WO 98/56748 provides higher processing stabilization, i.e., higher residual strength in a polypropylene than without its addition. A further improvement in processing stability is achieved by adding, in addition to 0.2% of the hexylester of vanilic acid, 0.2% mannitol or 0.2% tris-(2,4-di-tert-butylphenyl)phosphite.

Furthermore, the powder-powder mixtures were compounded and granulated together with 0.5% of the hexylester of vanilic acid by means of a twin-screw extruder (11 mm) at 210° C. The granulates underwent injection molding to produce test specimens, which were exposed to light in a weathering instrument (Bandol Wheel). While the test specimens without the additive already show chalkng of the surface, i.e., damage of the polymer, after 100 hours, the composition equipped with the inventive stabilizer is still unchanged after 300 hours.

This invention will be explained in detail on the basis of the following embodiments, however it is not limited to them.

### 1. General Synthesis Method for Preparation of the Inventive Phenolic Antioxidants and Light Stabilizers

The phenol ester (1 eq., 20 mmol) and the alcohol (0.3-1.5 eq., or 7-30 mmol) are placed in a dried Schlenk flask with a condensation bridge and cold trap. Under an inert atmosphere, the reactants are agitated and then briefly degassed. The tin catalyst (0.04 eq., 0.8 mmol) is added to the melt under nitrogen counterflow. The temperature is raised to 130° C.-140° C. and a slight vacuum is applied to the flask. The course of the reaction is checked using ¹H-NMR spectra. After the reaction has gone to completion, the temperature is raised to 150° C.-160° C. and the pressure is lowered to 1 mbar, to separate the unreacted reactants from the product. Here again, the progress is checked by means of a ¹H-NMR spectrum. After that, the vacuum is broken and the product is cooled to room temperature. The solid matter is taken up with dichloromethane and 2.8 g of bleaching clay are added. After 30 minutes of heating under reflux, the mixture is filtered through a short silica pad, and the dichloromethane is distilled off.

### 2. Detailed Synthesis Method: Preparation of Lauryl Vanillate

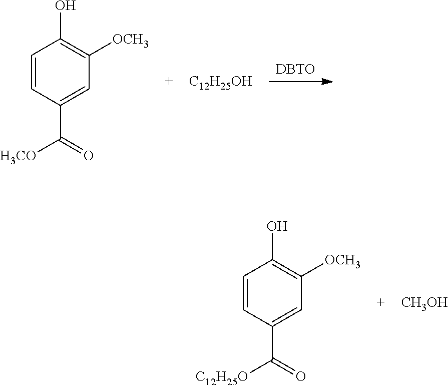

3.64 g of vanillinic acid methylester (methyl vanillate, 1 eq., 20 mmol) and 5.58 g of lauryl alcohol (1.5 eq., 30 mmol) are placed into a dried 200 mL Schlenk flask with a condensation bridge and a cold trap attached to it. The reactants are melted at a temperature of 90° C. under an inert nitrogen atmosphere and under agitation (250 rpm), and then briefly degassed. 199.15 mg of dibutyltin oxide (0.04 eq., 0.8 mmol) are added to the melt under nitrogen counterflow. The temperature is raised to 130° C. and a slight vacuum is applied to the flask (=800 mbar). In addition, the cold trap of the second flask is filled with liquid nitrogen, producing a slight negative pressure in the closed apparatus. The course of the reaction is checked using 1H-NMR spectra (formation of at triplet at 6=4.3 ppm). After the reaction has gone to completion, the temperature is raised to 150° C. and the pressure is lowered to 1 mbar, to separate the excess vanillic acid methyl ester from the product; here the progress is also checked by means of 1H-NMR spectrum (loss of the double peak at 6 3.8 ppm). After that, the vacuum is broken by introducing nitrogen, and the product is cooled to room temperature. The solid matter is taken up with dichloromethane and 2.8 g of bleaching clay (Optimum 210FF) are added. After 30 minutes of heating under reflux (oil bath temperature 60° C.), the mixture is filtered through a short silica pad, and the dichloromethane is distilled off under vacuum. The remaining solid matter is pulverised in a mortar and transferred to a snap-on cap bottle.

### 3. Products

The following products were synthesized in accordance with the general specification:

### Vanillate Structures:

Lauryl vanillate (LauVan), dodecyl 4-hydroxy-3-methoxybenzoates

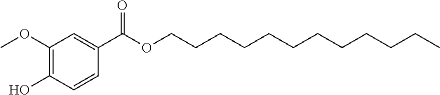 [SMILES: CCCCCCCCCCCCOC(=O)c1ccc(O)c(OC)c1]

Stearyl vanillate (SteaVan), octadecyl 4-hydroxy-3-methoxybenzoates

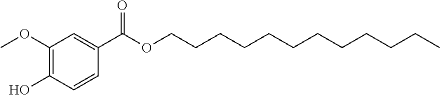 [SMILES: CCCCCCCCCCCCOC(=O)c1ccc(O)c(OC)c1]

Hexane divanillate (HexDiVan), hexane-1,6-diyl bis (4-hydroxy-3-methoxybenzoates)

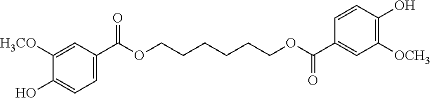 [SMILES: COc1cc(C(=O)OCCCCCCOC(=O)c2ccc(O)c(OC)c2)ccc1O]

Hexane trivanillate (HexTriVan), hexane-1,2,6-triyl tris (4-hydroxy-3-methoxybenzoates)

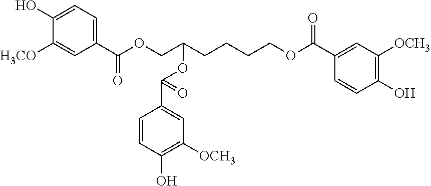 [SMILES: COc1cc(C(=O)OCCCCC(COC(=O)c2ccc(O)c(OC)c2)OC(=O)c2ccc(O)c(OC)c2)ccc1O]

Tetraethyleneglycylvanillate (TEGVan), ((oxybisethane-2,1-diyl))bis(oxy)bis(ethane-2,1-diyl) bis(4-hydroxy-3-methoxybenzoate)

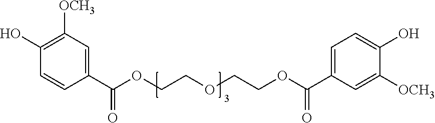

Octane divanillate (OctDiVan), octane-1,8-diyl bis(4-hydroxy-3-methoxybenzoate)

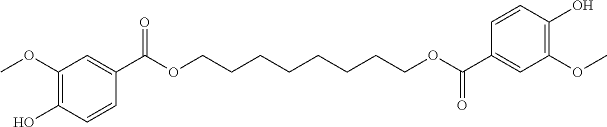

### Syringate Structures:

Lauryl syringate (LauSyr), dodecyl 4-hydroxy-3,5-dimethoxybenzoate

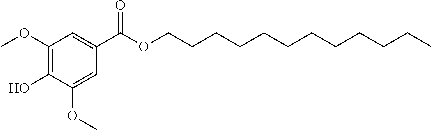 [SMILES: CCCCCCCCCCCCOC(=O)c1cc(OC)c(O)c(OC)c1]

Stearyl syringate (SteaSyr), octadecyl 4-hydroxy-3,5-dimethoxybenzoate

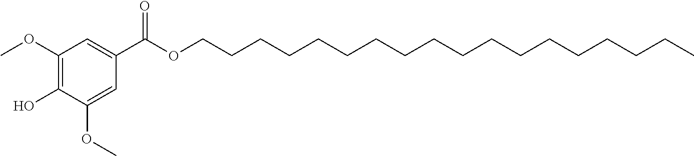 [SMILES: CCCCCCCCCCCCCCCCCCOC(=O)c1cc(OC)c(O)c(OC)c1]

Hexane disyringate (HexDiSyr), hexane-1,6-diyl bis (4-hydroxy-3,5-dimethoxybenzoate)

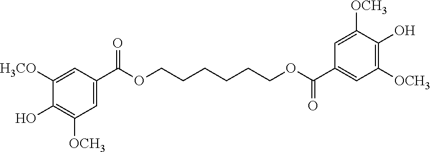 [SMILES: COc1cc(C(=O)OCCCCCCOC(=O)c2cc(OC)c(O)c(OC)c2)cc(OC)c1O]

Hexane trisyringate (HexTriSyr), hexane-1,2,6-triyl tris (4-hydroxy-3,5-dimethoxybenzoate)

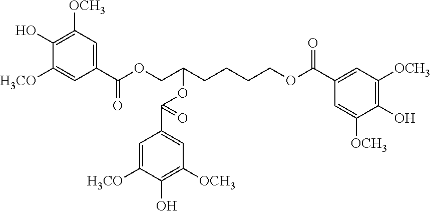 [SMILES: COc1cc(C(=O)OCCCCC(COC(=O)c2cc(OC)c(O)c(OC)c2)OC(=O)c2cc(OC)c(O)c(OC)c2)cc(OC)c1O]

N-SteaSyr, (4-hydroxy-3,5-dimethoxyphenyl)(octadecyl-12-azaneyl)methanone

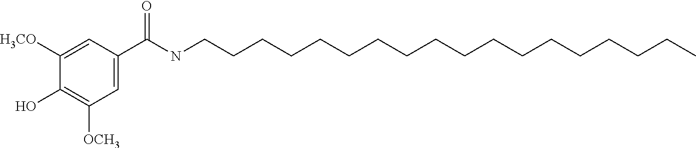 [SMILES: CCCCCCCCCCCCCCCCCCNC(=O)c1cc(OC)c(O)c(OC)c1]

### 4. Characteristics

The measured characteristics of the synthesized compounds are compiled in the following table:

| | Melting point | UV absorption maximum |
|---|---|---|
| Structure | /° C. | /nm |
| LauVan | 40 | 212 |
| SteaVan | 55 | 198 |
| HexDiVan | 101 | 202 |
| HexTriVan | 17 | 198 |
| TEGVan | −4 | 200 |
| OctDiVan | 115 | 200 |
| LauSyr | 60 | 198 |
| SteaSyr | ~30 | 198 |
| HexDiSyr | 140 | 218 |
| HexTriSyr | 74 | 222 |
| N-SteaSyr | 79 | 198 |

### Application Examples

In another series to test the efficacy of the inventive stabilizers in a commercially available polypropylene (Moplen HF 501N, Lyondell Basell Industries), selected synthesized compounds with a concentration of 0.035 mmol were homogenized in a powder-powder mixture, and cycled in a twin-screw microcompounder (MC 5, manufactured by DSM) for 30 minutes at 200° and 200 rpm, and the decrease in strength was recorded. The strength is a direct measure pf the molecular weight of polypropylene: the smaller the decrease, the higher the stabilization effect.

The data were evaluated as follows: The measured strengths are measured every five minutes. To even out fluctuations that occur during the measurement, the mean of the measured values is calculated every 30 seconds before the desired point. For comparability between the individual compounds, each of the measured values was standardized to the starting value. The percentage residual strength is indicated in each case.

| | Strength retained after minutes as a | | | | | |
|---|---|---|---|---|---|---|
| | percentage of the starting value | | | | | |
| | 5 | 10 | 15 | 20 | 25 | 30 |
| Comparison example 1 | 65 | 41 | 25 | 14 | 8 | 3 |
| (without additive) | | | | | | |
| Inventive example 1: | 73 | 55 | 40 | 28 | 19 | 13 |
| HexDiVan | | | | | | |
| Inventive example 2: | 90 | 81 | 65 | 50 | 37 | 26 |
| HexTriVan | | | | | | |
| Inventive example 3: | 66 | 48 | 34 | 24 | 16 | 11 |
| SteaVan | | | | | | |
| Inventive example 4: | 72 | 55 | 42 | 31 | 23 | 16 |
| TegVan | | | | | | |
| Inventive example 5: | 89 | 76 | 59 | 44 | 32 | 22 |
| SteaSyr | | | | | | |
| Inventive example 6: | 81 | 64 | 52 | 40 | 30 | 23 |
| HexTriSyr | | | | | | |
| Inventive example 7: | 90 | 88 | 85 | 81 | 76 | 67 |
| N-SteaSyr | | | | | | |

In all cases the inventive additives produce an improvement in stability over the comparison example.

1-16. (canceled)

17. A method of stabilizing a plastic against degradation comprising adding to the plastic one or more compounds having general formula I or II: 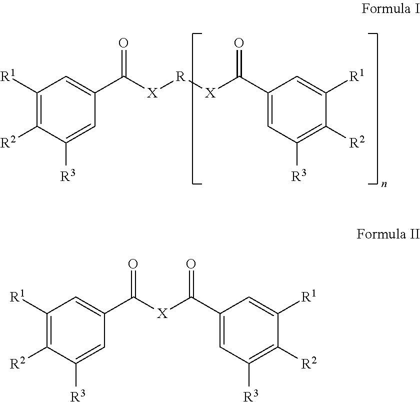
wherein:
R¹, R² and R³ are selected, each independently of one another, from the group consisting of hydroxyl, linear or branched alkoxy groups with 1 to 6 carbon atoms and hydrogen, with the condition that at least at least one of the groups R¹, R² and R³ is a hydroxyl group and at least one of the groups R¹, R² and R³ is a linear or branched alkoxy group with 1 to 6 carbon atoms, wherein in every occurrence, any of R¹, R² and R³ is the same or different,
R is an organic group, and
X upon every occurrence is the same or different and in formula I is O, NH, or NR, and in formula II it is O or NR, n is 0 or a whole number selected from 1 to 10.

18. The method of claim 17, wherein the stabilization is against oxidative, thermal and/or actinic degradation.

19. The method of claim 17, wherein R is selected from the group consisting of linear or branched and saturated or unsaturated alkyl groups or aromatic groups.

20. The method of claim 17, wherein:
R is a linear alkyl group with 6, 8, 9, 10, 11, 12, 13, 14, 16, 18, 20, 22, 24, 26, 28 or 30 carbon atoms, or
R is 11-methyldodecan-1-yl, 3,7-dimethyl-7-octen-1-yl, (R)-3,7-dimethyloct-6-en-1-yl, 2,6-dimethyl-2,6-octadien-8-yl, cis-9-hexadecen-1-yl, cis-9-octadecen-1-yl, cis-13-docosen-1-yl, cis,cis-9,12-octadecadien-1-yl, or 3,7-dimethyl-trans-2,6-octadien-1-yl; or
R is an aromatic group; or
R is an 2,3-dihydroxypropan-1-yl, or linear or branched, saturated or unsaturated alkanediyl group; or
R is a di-, tri- or tetravalent group derived from an alditol.

21. The method of claim 17, wherein the linear or branched, saturated or unsaturated alkanediyl group is 1,2-ethanediyl, 1,3-propanediyl, 1,4-butanediyl, 1,5-pentanediyl, 1,6-hexanediyl, or 1,8-octanediyl;

22. The method of claim 17, wherein R is linear or branched and saturated or unsaturated oxaalkanediyl groups.

23. The method of claim 17, wherein R is 3-oxapentane-1,5-diyl, 2,2′-(ethylenedioxy)diethanediyl, 2,4-dimethyl-3-oxapentane-1,5-diyl, 4-oxaheptane-2,6-diyl, 2-(2-hydroxypropyl)-1-propyl, 1,3-cyclohexanediyl, 1,4-cyclohexanediyl, benzene-1,4-diyl, benzene-1,3-diyl, 2-hydroxypropane-1,3-diyl, or 2-aminopropane-1,3-diyl.

24. The method of claim 17, wherein:
R¹ is a linear or branched alkoxyl group with 1 to 6 carbon atoms,
R² is hydroxy, and
R³ is a linear or branched alkoxy group with 1 to 6 carbon atoms, or hydrogen.

25. The method of claim 17, wherein the compound having general formula I is selected from the group consisting of 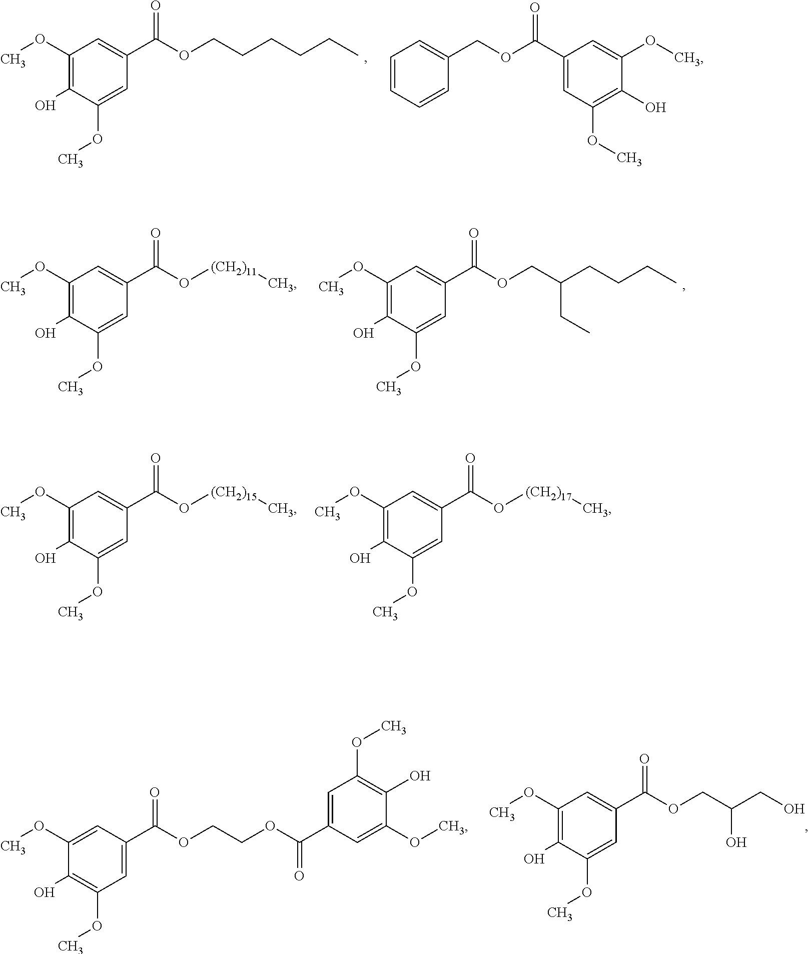 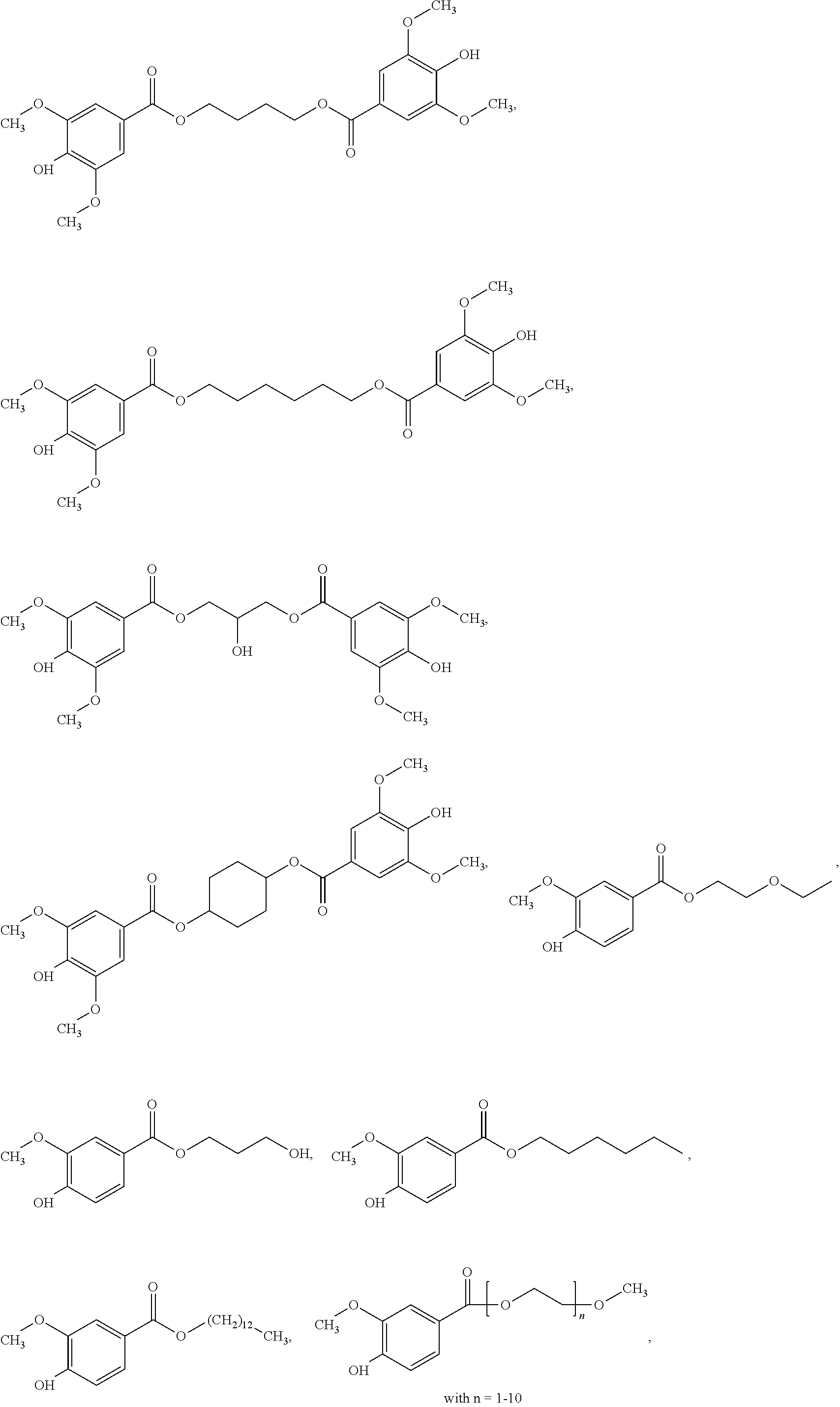 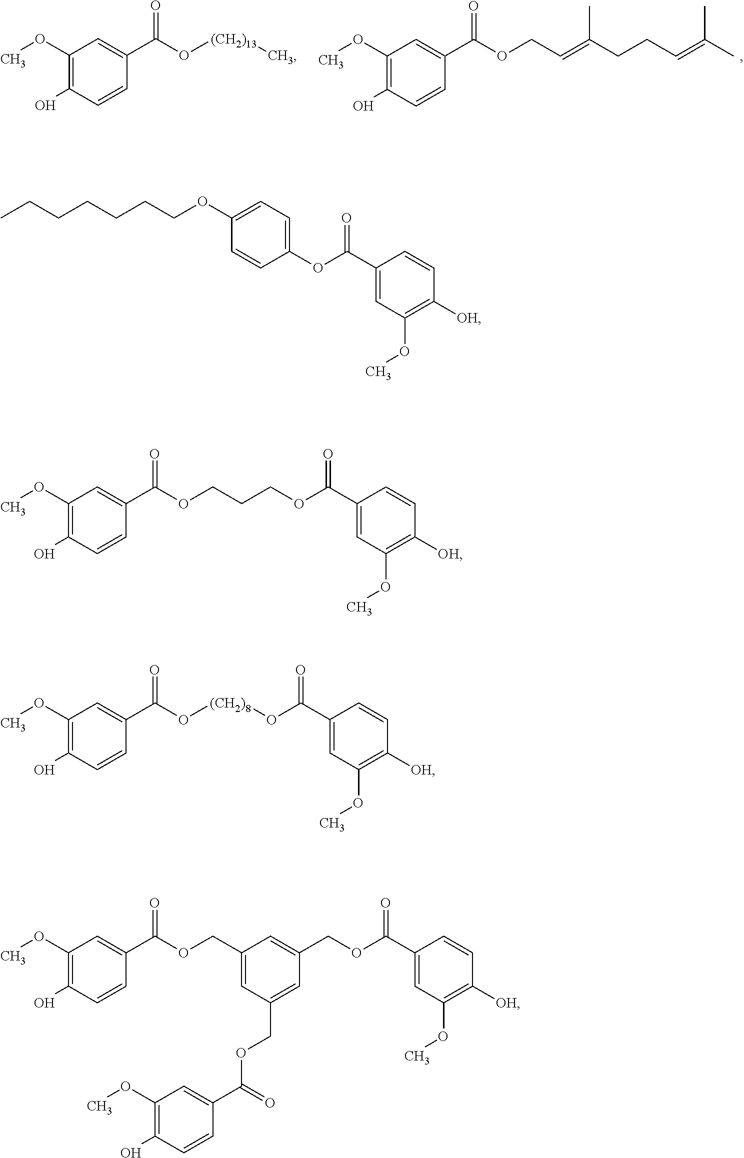 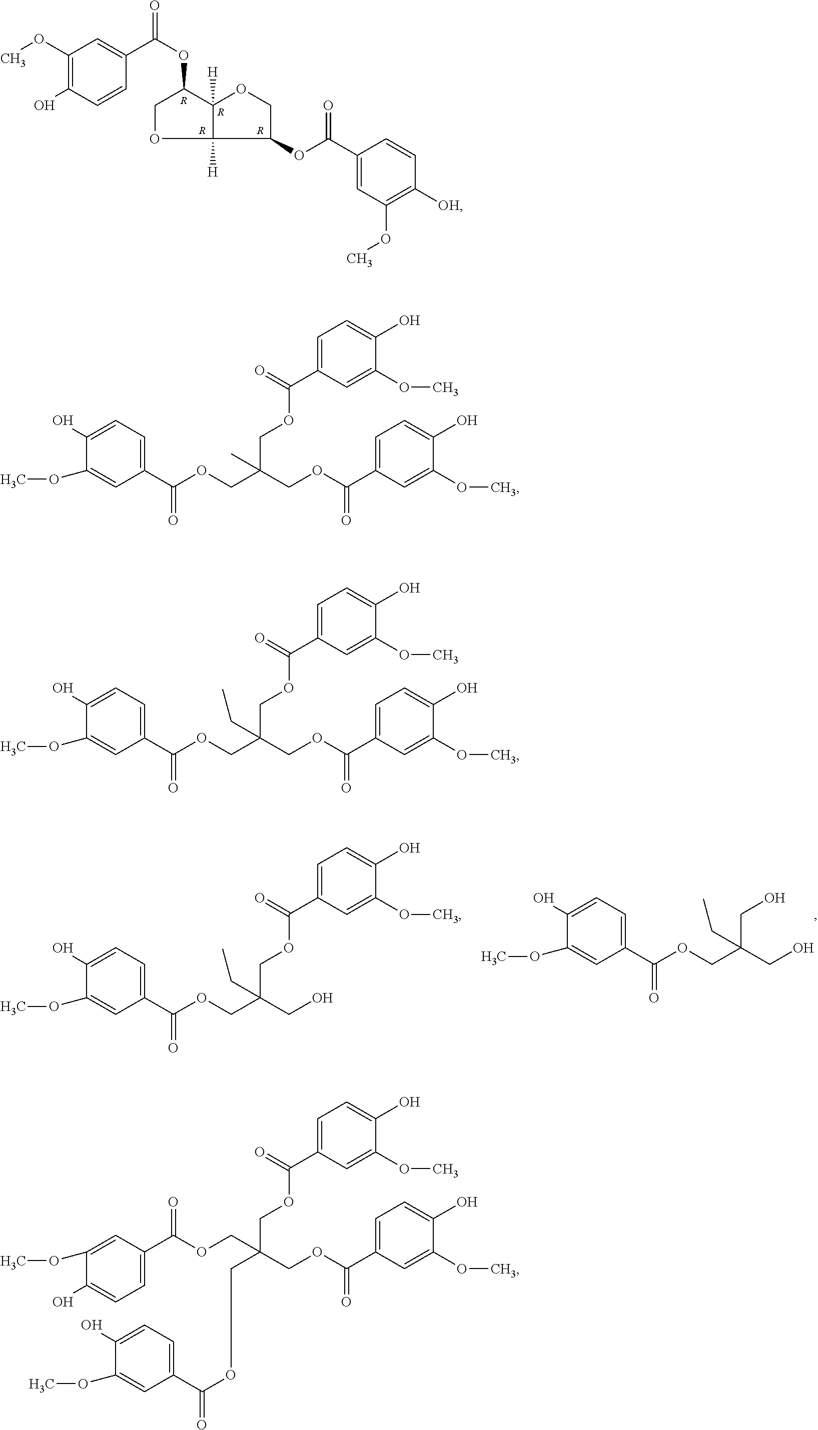 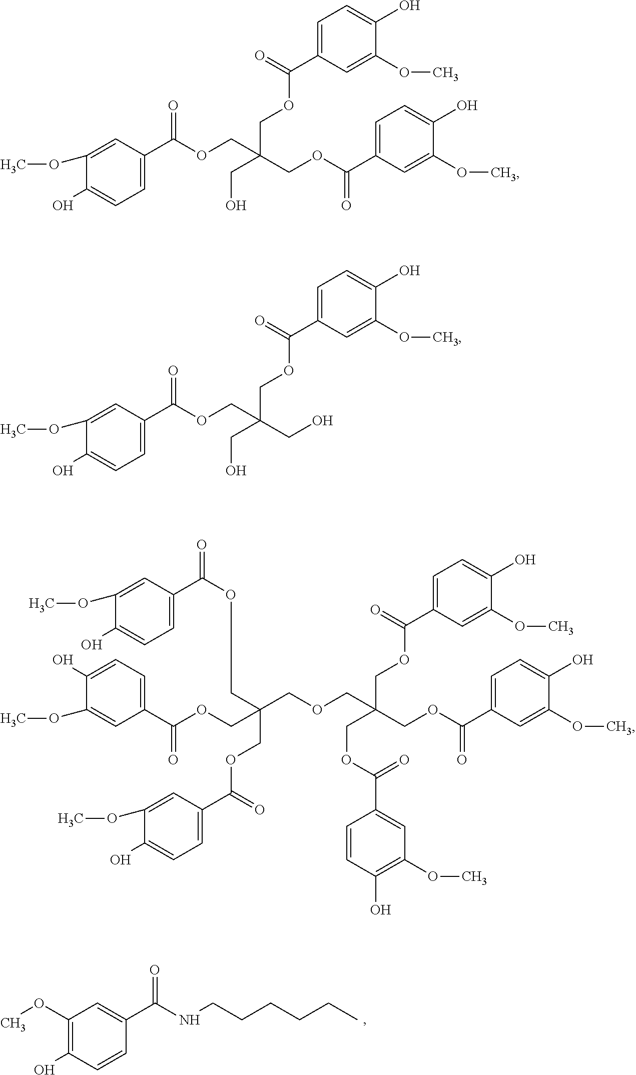 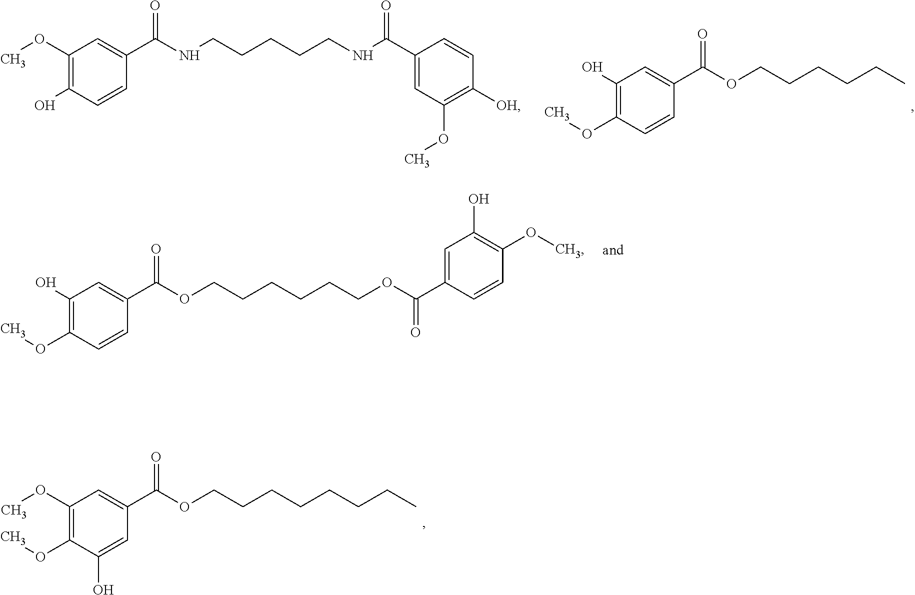
and the compound having general formula II is

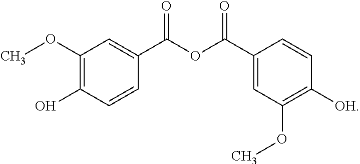

26. The method according to claim 17, wherein the compound having general formula I or II, or in the case of a mixture of multiple compounds having general formula I and/or II, all compounds having general formula I and/or II is/are contained in the plastic in a weight percentage from 0.01 to 10.00 weight %.

27. The method according to claim 17, wherein the plastic is selected from the group consisting of
(a) a polyethylene selected from the group consisting of LDPE, LLDPE, VLDPE, ULDPE, MDPE, HDPE, UHMWPE, and metallocene-PE (m-PE), polypropylene, polyisobutylene, poly-4-methyl-pentene-1, polybutadiene, polyisoprene, natural rubber (NR), poly(cyclooctene), polyalkylene-carbon monoxide copolymers, as well as copolymers in the form of statistical or block structures, ethylene acrylates, terpolymers, graft polymers, as well as blends thereof, and long-chain polypropylene copolymers that are produced with alpha olefins as comonomers,
(b) a styrenic polymer selected from the group consisting of polystyrene, polymethylstyrene, poly-alpha-methylstyrene, polyvinylnaphthalene, polyvinylbiphenyl, polyvinyl toluene, styrene-butadiene (SB), styrene-butadiene-styrene (SBS), styrene-ethylene-butylene-styrene (SEBS), styrene-ethylene-propylene styrene, styrene-isoprene, styrene-isoprene styrene (SIS), acrylonitrile butadiene styrene (ABS), styrene acrylonitrile (SAN), styrene acrylonitrile acrylate (ASA), styrene ethylene, styrene maleic acid anhydride polymers including corresponding graft polymers, and graft copolymers, and hydrogenated polystyrene derivatives,
(c) a halogen-containing polymer selected from the group consisting of polyvinyl chloride (PVC), polychloroprene, polyvinyldichloride (PVDC), copolymer of vinyl chloride and vinylidene chloride, copolymer of vinyl chloride and vinyl acetate, chlorinated polyethylene, polyvinylidene fluoride, epichlorohydrin homopolymer, and epichlorohydrin copolymers,
(d) a polymer of unsaturated ester selected from the group consisting of polymethylmethacrylate (PMMA), polybutylacrylate, polylaurylacrylate, polystearylacrylate, polyglycidylacrylate, and polyglycidylmethacrylate, polyacrylonitrile, polyacrylamide, or a copolymer of acrylonitrile and acrylamide,
(e) polyvinyl alcohol, polyvinyl acetate, polyvinyl butyral, polyallyl phthalate, or polyallyl melamine,
(f) a polyacetalene or a copolymer thereof,
(g) polyphenylene oxides or a blend thereof with polystyrene or a polyamide,
(h) polymers of cyclic ethers,
(i) polyurethanes of hydroxy-terminated polyethers or polyesters and aromatic or aliphatic isocyanates,
(j) polyamides or blends of polyamides and polyolefins,
(k) plastics selected from the group consisting of polyimides, polyamide imides, polyether imides, polyester imides, poly(ether)ketones, polysulfones, polyether sulfones, polyaryl sulfones, poly(phenylene sulfides), poly(benzimidazoles), and polyhydantoins,
(l) polyesters of aliphatic or aromatic dicarboxylic acides and diols or of hydroxy-carboxylic acids,
(m) polycarbonates, polyester carbonates, and blends one or more of these polymers with other polymers,
(n) cellulose nitrate, cellulose acetate, cellulose propionate, or cellulose butyrate,
(o) epoxy resin consisting of di- or polyfunctional epoxides in combination with hardeners based on amines, anhydrides, dicyandiamide, mercaptans, isocyanates or catalytically active hardeners,
(p) phenolic resins,
(q) unsaturated polyester resins made of unsaturated dicarboxylic acids and diols with vinyl compounds,
(r) silicones,
and mixtures, combinations or blends thereof.

28. The method according to claim 17, which includes adding at least one further additive to the plastic, wherein the at least one further additive is selected from the group consisting of primary and/or secondary antioxidants, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents, and antifogging agents.

29. The method according to claim 28, wherein the at least one further additive is selected from the group consisting of phosphites, phosphonites, sulfites, polyols, acid scavengers, hindered amines, and mixtures and combinations thereof.

30. The method according to claim 28, wherein the at least one further additive is added in a quantity from 0.01 to 80 weight, relative to the total of the at least one compound having general formula I or II, or in the case of a mixture of multiple compounds having general formula I and/or II, the sum of all compounds having general formula I and/or II, of the plastic, and of the at least one additive.

31. A plastic composition comprising at least one plastic and at least one compound having general formula I and/or II or mixtures of multiple compounds having general formula I and/or II as defined in claim 17.

32. The plastic composition according to claim 31, with following composition:
0.01 to 10.00 weight of a compound having general formula I or II or, in the case of a mixture of multiple compounds having general formula I and/or II, the sum of all compounds having general formula I and/or II,
99.99 to 10.00, weight % of at least one plastic, and
0 to 80.00 weight-% of at least one additive,
wherein components of the composition add up to 100 weight %.

33. The plastic composition according to claim 32, wherein the at least one additive is selected from the group consisting of primary and secondary antioxidants.

34. The plastic composition according to claim 32, wherein the at least one additive is selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents, and antifogging agents.

35. A compound having one of the following formulas:

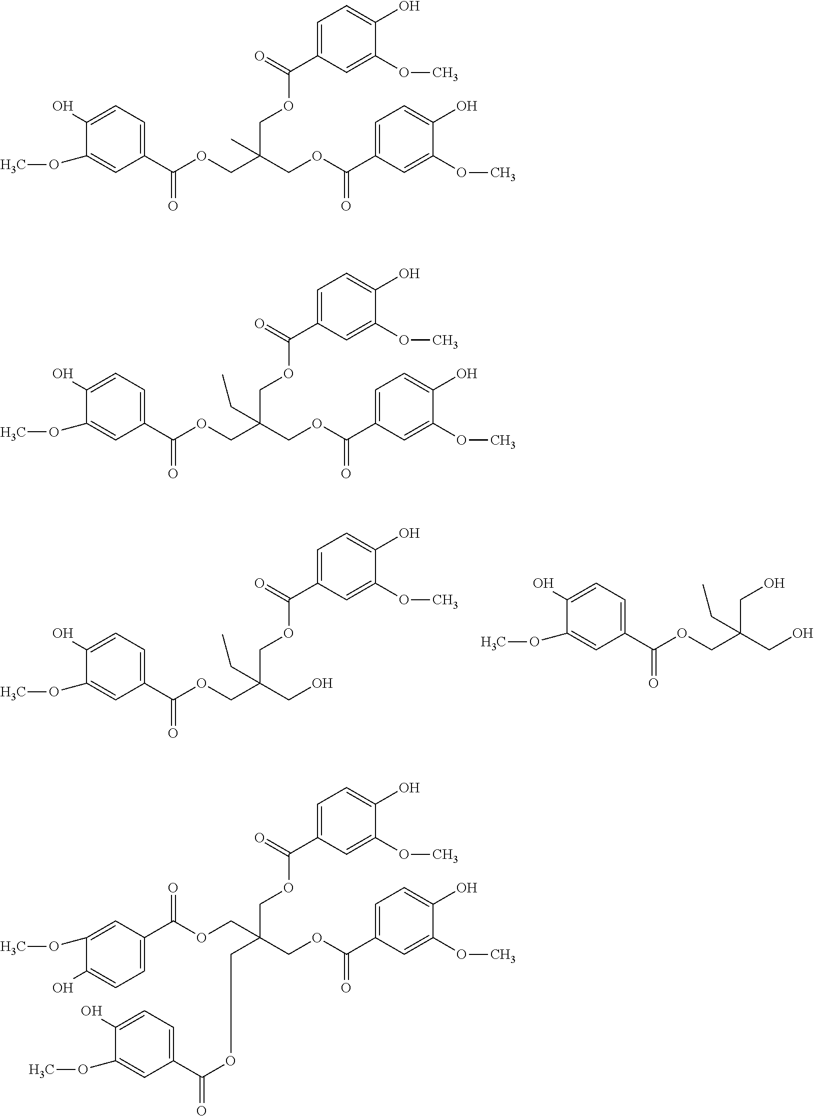

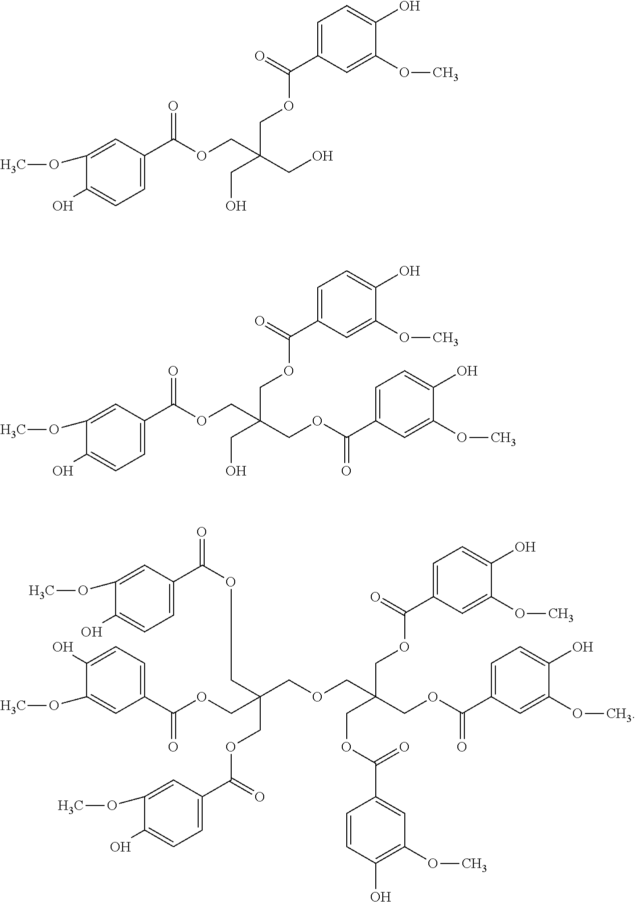

36. A stabilizer composition consisting of at least one compound having general formula I or II or mixtures of multiple compounds having general formula I and/or as defined in claim 17 (component A), and at least one additive (component B), selected from the group consisting of primary and/or secondary antioxidants, UV absorbers, light stabilizers, hydroxylamine-based stabilizers, benzofuranone-based stabilizers, nucleation agents, tougheners, plasticizers, lubricants, rheology modifiers, chain extenders, processing aids, pigments, dyes, brighteners, antimicrobials, antistatic agents, slip agents, antiblocking agents, coupling agents, dispersants, compatibilizers, oxygen scavengers, acid scavengers, costabilizers, marking agents, and antifogging agents.

37. The stabilizer composition according to claim 36, wherein component A and component B are in a weight ratio of 100:1 to 1:100.
